(19) United States
(12) Patent Application Publication
(10) Pub. No.: US 2024/0417423 A1
(43) Pub. Date: Dec. 19, 2024

(54) DEUTERATED POSITIVE NMDA-MODULATING COMPOUNDS AND METHODS OF USE THEREOF

(71) Applicant: SAGE THERAPEUTICS, INC., Cambridge, MA (US)

(72) Inventor: Maria-Jesus Blanco-Pillado, Cambridge, MA (US)

(21) Appl. No.: 18/694,572

(22) PCT Filed: Sep. 22, 2022
(86) PCT No.: PCT/US2022/044447

§ 371 Date: Mar. 22, 2024

Related U.S. Application Data
(60) Provisional application No. 63/247,052, filed on Sep. 22, 2021.

(51) Int. Cl. C07J 9/00; A61K 31/575; C07B 59/00

(52) U.S. Cl. CPC C07J 9/00; A61K 31/575; C07B 59/007

(57) ABSTRACT

Deuterated compounds are provided according to Formula (I): Formula (I) and pharmaceutically acceptable salts thereof, and pharmaceutical compositions thereof. These compounds are contemplated as useful for the prevention and treatment of a variety of CNS-related conditions.

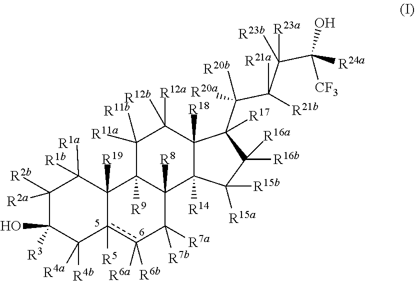

### CROSS REFERENCE TO RELATED APPLICATIONS

This application claims priority to and the benefit of U.S. Provisional Application No. 63/247,052, filed Sep. 22, 2021, the disclosure of which is incorporated by reference herein in its entirety.

### BACKGROUND

NMDA receptors are heteromeric complexes comprised of NR1, NR2, and/or NR3 subunits and possess distinct recognition sites for exogenous and endogenous ligands. These recognition sites include binding sites for glycine, and glutamate agonists and modulators. NMDA receptors are expressed in the peripheral tissues and the CNS, where they are involved in excitatory synaptic transmission. Activating these receptors contributes to synaptic plasticity in some circumstances and excitotoxicity in others. These receptors are ligand-gated ion channels that admit Ca²⁺ after binding of the glutamate and glycine, and are fundamental to excitatory neurotransmission and normal CNS function. Positive modulators may be useful as therapeutic agents with potential clinical uses as cognitive enhancers and in the treatment of psychiatric disorders in which glutamatergic transmission is reduced or defective (see, e.g., Horak et al., J. of Neuroscience, 2004, 24(46), 10318-10325).

There is a need for new compounds that are positive allosteric modulators of the NMDA receptor for the prevention and treatment of conditions associated with NMDA function. Compounds, compositions, and methods described herein are directed toward this end.

### SUMMARY

Provided herein are compounds designed, for example, to act as NMDA positive allosteric modulators. The compounds of the Formula (I) are compounds that comprise deuterium levels that are above the naturally-occurring levels. In some embodiments, such compounds are envisioned to be useful as therapeutic agents for treating a CNS-related disorder.

In one aspect, the disclosure provides a compound of Formula (I):

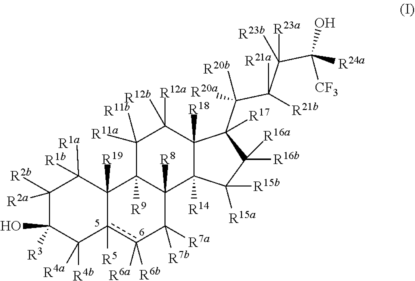

or a pharmaceutically acceptable salt thereof;
wherein:
each of R³, R¹⁸, R¹⁹, R^{20a} and R^{24a} is independently C₁₋₃alkyl optionally substituted with 1 or more independent occurrences of deuterium;
each of R^{1a}, R^{1b}, R^{2a}, R^{2b}, R^{4a}, R^{4b}, R⁵, R^{6a}, R^{6b}, R^{7a}, R^{7b}, R⁸, R⁹, R^{11a}, R^{11b}, R^{12a}, R^{12b}, R¹⁴, R^{15a}, R^{15b}, R^{16a}, R^{16b}, R¹⁷, R^{20b}, R^{21a}, R^{21b}, R^{23a}, and, R^{23b} is independently selected from a group consisting of hydrogen and deuterium;
provided that at least one of R³, R¹⁸, R¹⁹, R^{20a} and R^{24a} is substituted with 1 or more independent occurrences of deuterium, or at least one of R^{1a}, R^{1b}, R^{2a}, R^{2b}, R^{4a}, R^{4b}, R⁵, R^{6a}, R^{6b}, R^{7a}, R^{7b}, R⁸, R⁹, R^{11a}, R^{11b}, R^{12a}, R^{12b}, R¹⁴, R^{15a}, R^{15b}, R^{16a}, R^{16b}, R¹⁷, R^{20b}, R²¹, R^{21b}, R^{23a}, and R^{23b} is deuterium;
[symbol] represents a single or double bond; provided if the bond between C5 and C6 is a single bond, then R⁵ is in the alpha configuration.

In some embodiments, the compound of Formula (I) a compound of Formula (Ia):

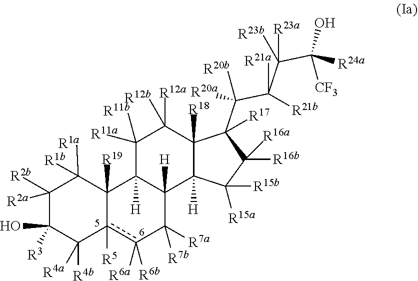

or a pharmaceutically acceptable salt thereof;
wherein:
each of R³, R¹⁸, R¹⁹, R^{20a} and R^{24a} is independently C₁₋₃alkyl optionally substituted with 1 or more independent occurrences of deuterium;
each of R^{1a}, R^{1b}, R^{2a}, R^{2b}, R^{4a}, R^{4b}, R⁵, R^{6a}, R^{6b}, R^{7a}, R^{7b}, R^{11a}, R^{11b}, R^{12a}, R^{12b}, R^{15a}, R^{15b}, R^{16a}, R^{16b}, R¹⁷, R^{20b}, R^{21a}, R^{21b}, R^{23a}, and, R^{23b} is independently selected from a group consisting of hydrogen and deuterium;
provided that at least one of R³, R¹⁸, R¹⁹, R^{20a} and R^{24a} is substituted with 1 or more independent occurrences of deuterium, or at least one of R^{1a}, R^{1b}, R^{2a}, R^{2b}, R^{4a}, R^{4b}, R⁵, R^{6a}, R^{6b}, R^{7a}, R^{7b}, R^{11a}, R^{11b}, R^{12a}, R^{12b}, R^{15a}, R^{15b}, R^{16a}, R^{16b}, R¹⁷, R^{20b}, R^{21a}, R^{21b}, R^{23a}, and R^{23b} is deuterium;
[symbol] represents a single or double bond; provided if the bond between C5 and C6 is a single bond, then R⁵ is in the alpha configuration.

In some embodiments, the compound of formula (Ia) is a compound of formula (II):

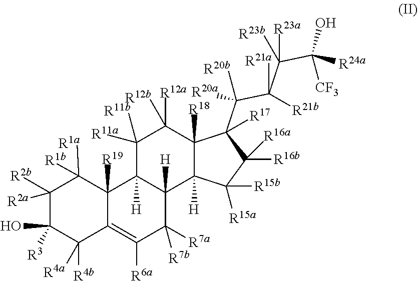

or a pharmaceutically acceptable salt thereof.

In some embodiments, the compound of formula (Ia) is a compound of formula (III):

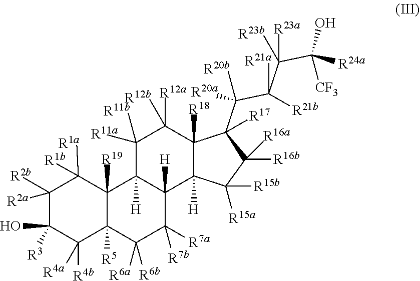

or a pharmaceutically acceptable salt thereof.

In some embodiments, the disclosure provides a compound as disclosed herein. In some embodiments, the disclosure provides a compound of Formula (I). In some embodiments, the disclosure provides a compound of Formula (Ia). In some embodiments, the disclosure provides a compound of Formula (II). In some embodiments, the disclosure provides a compound of Formula (III). In some embodiments, the disclosure provides a compound of Formula (IV). In some embodiments, the disclosure provides a compound of Formula (V). In some embodiments, the disclosure provides a pharmaceutically acceptable salt of a compound disclosed herein. In some embodiments, the disclosure provides a pharmaceutically acceptable salt of a compound of Formula (I). In some embodiments, the disclosure provides a pharmaceutically acceptable salt of a compound of Formula (Ia). In some embodiments, the disclosure provides a pharmaceutically acceptable salt of a compound of Formula (II). In some embodiments, the disclosure provides a pharmaceutically acceptable salt of a compound of Formula (III). In some embodiments, the disclosure provides a pharmaceutically acceptable salt of a compound of Formula (IV). In some embodiments, the disclosure provides a pharmaceutically acceptable salt of a compound of Formula (V).

In some embodiments, at least one of R^{3a}, R^{3b}, R¹⁹, R^{20a}, or R^{24a} is C₁₋₃alkyl substituted with 1 or more independent occurrences of deuterium, or at least one of R^{2a}, R^{2b}, R^{4a}, R^{4b}, R⁵, R^{6a}, R^{7a}, R^{7b}, R^{11a}, R^{11b}, R^{15a} and R^{15b} is deuterium. In some embodiments, at least one of R^{3a}, R¹⁸, R¹⁹, R^{20a}, or R^{24a} is C₁₋₃alkyl substituted with 1 or more independent occurrences of deuterium. In some embodiments, at least one of R^{2a}, R^{2b}, R^{4a}, R^{4b}, R⁵, R^{6a}, R^{7a}, R^{7b}, R^{11a}, R^{11b}, R^{15a} and R^{15b} is deuterium.

In some embodiments, each of R^{1a} and R^{1b} is independently hydrogen or deuterium. In some embodiments, each of R^{1a} and R^{1b} is independently deuterium. In some embodiments, each of R^{1a} and R^{1b} is independently hydrogen.

In some embodiments, each of R^{2a} and R^{2b} is independently hydrogen or deuterium. In some embodiments, each of R^{2a} and R^{2b} is independently deuterium. In some embodiments, each of R^{2a} and R^{2b} is independently hydrogen.

In some embodiments, R³ is C₁₋₃alkyl optionally substituted with 1 or more independent occurrences of deuterium. In some embodiments, R³ is C₁₋₃alkyl substituted with 1 or more independent occurrences of deuterium. In some embodiments, R³ is methyl substituted with 1-3 independent occurrences of deuterium. In some embodiments, R³ is —CH₃. In some embodiments, R³ is —CD₃.

In some embodiments, each of R^{4a} and R^{4b} is independently hydrogen or deuterium. In some embodiments, each of R^{4a} and R^{4b} is independently deuterium. In some embodiments, each of R^{4a} and R^{4b} is independently hydrogen.

In some embodiments, R⁵ is hydrogen or deuterium. In some embodiments, R⁵ is deuterium. In some embodiments, R⁵ is hydrogen. In some embodiments, each of R⁵ and R^{6a} is independently hydrogen or deuterium. In some embodiments, each of R⁵ and R^{6a} is independently deuterium. In some embodiments, each of R⁵ and R^{6a} is independently hydrogen.

In some embodiments, each of R^{6a} and R^{6b} is independently hydrogen or deuterium. In some embodiments, each of R^{6a} and R^{6b} is independently deuterium. In some embodiments, each of R^{6a} and R^{6b} is independently hydrogen.

In some embodiments, each of R^{7a} or R^{7b} is independently hydrogen or deuterium. In some embodiments, each of R^{7a} or R^{b} is independently deuterium. In some embodiments, each of R^{7a} or R^{7b} is independently hydrogen.

In some embodiments, R⁸ is hydrogen or deuterium. In some embodiments, R⁸ is deuterium. In some embodiments, R⁸ is hydrogen.

In some embodiments, R⁹ is hydrogen or deuterium. In some embodiments, R⁹ is deuterium. In some embodiments, R⁹ is hydrogen.

In some embodiments, each of R^{11a} and R^{11b} is independently hydrogen or deuterium. In some embodiments, each of R^{11a} and R^{11b} is independently deuterium. In some embodiments, each of R^{11a} and R^{11b} is independently hydrogen.

In some embodiments, each of R^{12a} and R^{12b} is independently hydrogen or deuterium. In some embodiments, each of R^{12a} and R^{12b} is independently deuterium. In some embodiments, each of R^{12a} and R^{12b} is independently hydrogen.

In some embodiments, R¹⁴ is hydrogen or deuterium. In some embodiments, R¹⁴ is deuterium. In some embodiments, R¹⁴ is hydrogen.

In some embodiments, each of R^{15a} and R^{15b} is independently hydrogen or deuterium. In some embodiments, each of R^{15a} and R^{15b} is independently deuterium. In some embodiments, each of R^{15a} and R^{15b} is independently hydrogen.

In some embodiments, each of R^{16a} and R^{16b} is independently hydrogen or deuterium. In some embodiments, each of R^{16a} and R^{16b} is independently deuterium. In some embodiments, each of R^{16a} and R^{16b} is independently hydrogen.

In some embodiments, R¹⁷ is hydrogen or deuterium. In some embodiments, R¹⁷ is hydrogen or deuterium. In some embodiments, R¹⁷ is deuterium. In some embodiments R¹⁷ is hydrogen.

In some embodiments, R¹⁸ is C₁₋₃alkyl optionally substituted with 1 or more independent occurrences of deuterium. In some embodiments, R¹⁸ is C₁₋₃alkyl substituted with 1 or more independent occurrences of deuterium. In some embodiments, R¹⁸ is methyl substituted with 1-3 independent occurrences of deuterium. In some embodiments, R¹⁸ is —CD₃. In some embodiments, R¹⁸ is —CH₃.

In some embodiments, R¹⁹ is C₁₋₃alkyl optionally substituted with 1 or more independent occurrences of deuterium. In some embodiments, R¹⁹ is C₁₋₃alkyl substituted with 1 or more independent occurrences of deuterium. In some embodiments, R¹⁹ is methyl substituted with 1-3 independent occurrences of deuterium. In some embodiments, R¹⁹ is —CD₃. In some embodiments, R¹⁹ is —CH₃.

In some embodiments, R^{20a} is C₁₋₃alkyl optionally substituted with 1 or more independent occurrences of deuterium. In some embodiments, R^{20a} is C₁₋₃alkyl substituted with 1 or more independent occurrences of deuterium. In some embodiments, R^{2a} is methyl substituted with 1-3 independent occurrences of deuterium. In some embodiments, R^{20a} is —CD₃. In some embodiments, R^{20a} is —CH₃. In some embodiments, R^{20b} is deuterium. In some embodiments, R^{20b} is hydrogen.

In some embodiments, each of R^{21a} and R^{21b} is independently hydrogen or deuterium. In some embodiments, each of R^{21a} and R^{21b} is independently deuterium. In some embodiments, each of R^{21a} and R^{21b} is independently hydrogen.

In some embodiments, each of R^{23a} and R^{23b} is independently hydrogen or deuterium. In some embodiments, each of R^{23a} and R^{23b} is independently deuterium. In some embodiments, each of R^{23a} and R^{23b} is independently hydrogen.

In some embodiments, R^{24a} is C₁₋₃alkyl optionally substituted with 1 or more independent occurrences of deuterium. In some embodiments, R^{24a} is C₁₋₃alkyl substituted with 1 or more independent occurrences of deuterium. In some embodiments, R^{24a} is methyl substituted with 1-3 independent occurrences of deuterium. In some embodiments, R^{24a} is —CD₃. In some embodiments, R^{24a} is —CH₃.

In one aspect, the disclosure provides a pharmaceutical composition comprising a compound or pharmaceutically acceptable salt thereof according to the disclosure, and a pharmaceutically acceptable carrier. In some embodiments, the disclosure provides a pharmaceutical composition comprising a compound according to the disclosure, and a pharmaceutically acceptable carrier. In some embodiments, the disclosure provides a pharmaceutical composition comprising a pharmaceutically acceptable salt of a compound according to the disclosure, and a pharmaceutically acceptable carrier.

In one aspect, the disclosure provides a method for treating a CNS-related condition in a subject, comprising administering to the subject an effective amount of a compound or pharmaceutically acceptable salt thereof according to the disclosure, or a pharmaceutical composition according to the disclosure. In some embodiments, the disclosure provides a method for treating a CNS-related condition in a subject, comprising administering to the subject an effective amount of a compound according to the disclosure, or a pharmaceutical composition comprising a compound according to the disclosure. In some embodiments, the disclosure provides a method for treating a CNS-related condition in a subject, comprising administering to the subject an effective amount of a pharmaceutically acceptable salt of compound according to the disclosure, or a pharmaceutical composition comprising a pharmaceutically acceptable salt of compound according to the disclosure.

In one aspect, the disclosure provides a method of inducing sedation or anesthesia in a subject, comprising administering to the subject an effective amount of a compound or pharmaceutically acceptable salt thereof according to the disclosure, or a pharmaceutical composition according to the disclosure. In some embodiments, the disclosure provides a method of inducing sedation or anesthesia in a subject, comprising administering to the subject an effective amount of a compound according to the disclosure, or a pharmaceutical composition comprising a compound according to the disclosure. In some embodiments, the disclosure provides a method of inducing sedation or anesthesia in a subject, comprising administering to the subject an effective amount of a pharmaceutically acceptable salt of compound according to the disclosure, or a pharmaceutical composition comprising a pharmaceutically acceptable salt of compound according to the disclosure.

In one aspect, the disclosure provides a compound or pharmaceutically acceptable salt thereof according to the disclosure, or a pharmaceutical composition according to the disclosure, for use in treating a CNS-related condition in a subject. In some embodiments, the disclosure provides a compound according to the disclosure, or a pharmaceutical composition comprising a compound according to the disclosure, for use in treating a CNS-related condition in a subject. In some embodiments, the disclosure provides a pharmaceutically acceptable salt of a compound according to the disclosure, or a pharmaceutical composition comprising a pharmaceutically acceptable salt of a compound according to the disclosure, for use in treating a CNS-related condition in a subject.

In one aspect, the disclosure provides a compound or pharmaceutically acceptable salt thereof according to the disclosure, or a pharmaceutical composition according to the disclosure, for use inducing sedation or anesthesia in a subject. In some embodiments, the disclosure provides a compound according to the disclosure, or a pharmaceutical composition comprising a compound according to the disclosure, for use inducing sedation or anesthesia in a subject. In some embodiments, the disclosure provides a pharmaceutically acceptable salt of a compound according to the disclosure, or a pharmaceutical composition comprising a pharmaceutically acceptable salt of a compound according to the disclosure, for use inducing sedation or anesthesia in a subject.

In one aspect, the disclosure provides a use of a compound or pharmaceutically acceptable salt thereof according to the disclosure, or a pharmaceutical composition according to the disclosure, for the manufacture of a medicament for treating a CNS-related condition in a subject. In some embodiments, the disclosure provides a use of a compound according to the disclosure, or a pharmaceutical composition comprising a compound according to the disclosure, for the manufacture of a medicament for treating a CNS-related condition in a subject. In some embodiments, the disclosure provides a use of a pharmaceutically acceptable salt of a compound according to the disclosure, or a pharmaceutical composition comprising a pharmaceutically acceptable salt of a compound according to the disclosure, for the manufacture of a medicament for treating a CNS-related condition in a subject.

In one aspect, the disclosure provides a use of a compound or pharmaceutically acceptable salt thereof according to the disclosure, or a pharmaceutical composition according to the disclosure, for the manufacture of a medicament for inducing sedation or anesthesia in a subject. In some embodiments, the disclosure provides a use of a compound according to the disclosure, or a pharmaceutical composition comprising a compound according to the disclosure, for the manufacture of a medicament for treating a CNS-related condition in a subject. In some embodiments, the disclosure provides a use of a pharmaceutically acceptable salt of a compound according to the disclosure, or a pharmaceutical composition comprising a pharmaceutically acceptable salt of a compound according to the disclosure, for the manufacture of a medicament for treating a CNS-related condition in a subject.

In some embodiments, the CNS-related condition is selected from the group consisting of an adjustment disorder, anxiety disorder (including obsessive-compulsive disorder, post-traumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

### DETAILED DESCRIPTION

The present disclosure provides compounds that NMDA positive allosteric modulators. The compounds of the disclosure are useful as therapeutic agents for treating CNS-related conditions including, but not limited to adjustment disorders, an anxiety disorders, cognitive disorders, dissociative disorders, eating disorders, mood disorders, schizophrenia or other psychotic disorders, sleep disorders, substance-related disorder, personality disorder, autism spectrum disorders, neurodevelopmental disorder, pain, encephalopathy secondary to a medical condition, seizure disorders, stroke, traumatic brain injury, movement disorders and tinnitus.

### General Definitions

The term “herein” means the entire application.

Unless otherwise defined herein, scientific and technical terms used in this application shall have the meanings that are commonly understood by those of ordinary skill in the art to which this disclosure belongs. Generally, nomenclature used in connection with the compounds, composition and methods described herein, are those well-known and commonly used in the art.

It should be understood that any of the embodiments described herein, including those described under different aspects of the disclosure and different parts of the specification (including embodiments described only in the Examples) can be combined with one or more other embodiments of the disclosure, unless explicitly disclaimed or improper. Combination of embodiments are not limited to those specific combinations claimed via the multiple dependent claims. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

Throughout this specification, the word “comprise” or variations such as “comprises” or “comprising,” which is synonymous with “including,” “containing,” or “characterized by,” will be understood to imply the inclusion of a stated integer (or component, element, or method) or group of integers (or components, elements, or methods), but not the exclusion of any other integer (or component, element or method) or group of integers (or components, elements, or methods). It is also noted that the terms “comprising” and “containing” are intended to be open and permits the inclusion of additional elements or steps.

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “including,” as used herein, means “including but not limited to.” “Including” and “including but not limited to” are used interchangeably. Thus, these terms will be understood to imply the inclusion of a stated integer (or component, element or method) or group of integers (or components, elements or methods), but not the exclusion of any other integer (or component, element or method) or group of integers (or components, elements or methods).

As used herein, “about” or “approximately” means within an acceptable error range for the particular value as determined by one of ordinary skill in the art, which will depend in part on how the value is measured or determined, i.e., the limitations of the measurement system.

The use of the terms “a” and “an” and “the” and similar referents in the context of describing the elements (especially in the context of the following claims) are to be construed to cover both the singular and the plural, unless otherwise indicated herein or clearly contradicted by context. By way of example “an analogue” means one analogue or more than one analogue.

The term “or” as used herein should be understood to mean “and/or,” unless the context clearly indicates otherwise.

Recitation of ranges of values herein are merely intended to serve as a shorthand method of referring individually to each separate value falling within the range and including the endpoints, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

All of the publications, patents and published patent applications referred to in this application are specifically incorporated by reference herein. In case of conflict, the present specification, including its specific definitions, will control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

### Chemical Definitions

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

Compounds described herein (e.g., a compound of Formula (I), e.g., a compound of Formula (I)) are deuterium-enriched.

Deuterium (D or 2H) is a stable, non-radioactive isotope of hydrogen and has an atomic weight of 2.0144. Hydrogen naturally occurs as a mixture of the isotopes 1H (hydrogen or protium), D (2H or deuterium), and T (3H or tritium). The natural abundance of deuterium is 0.015%. One of ordinary skill in the art recognizes that in all chemical compounds with a H atom, the H atom actually represents a mixture of H and D, with about 0.015% being D. Thus, compounds with a level of deuterium that has been enriched to be greater than its natural abundance of 0.015% should be considered unnatural and, as a result, novel over their non-enriched counterparts.

The effects of deuterium modification on a compound's metabolic properties are not predictable, even when deuterium atoms are incorporated at known sites of metabolism. Only by actually preparing and testing a deuterated compound can one determine if and how the rate of metabolism will differ from that of its non-deuterated counterpart. See, for example, Fukuto et al. (J. Med. Chem. 1991, 34, 2871-76). Many compounds have multiple sites where metabolism is possible. The site(s) where deuterium substitution is required and the extent of deuteration necessary to see an effect on metabolism, if any, will be different for each compound.

Unless otherwise stated, when a position is designated specifically as “H” or “hydrogen,” the position is understood to have hydrogen (i.e., 1H) at its natural abundance isotopic composition. Also unless otherwise stated, when a position is designated specifically as “D” or “deuterium,” the position is understood to have deuterium (i.e., 2H) at an abundance that is at least 3000 times greater than the natural abundance of deuterium, which is 0.015% (i.e., the term “D” or “deuterium” indicates at least 45% incorporation of deuterium).

The term “isotopic enrichment factor” as used herein means the ratio between the isotopic abundance of D at the specified position in a compound of this invention and the naturally occurring abundance of that isotope. Increasing the amount of deuterium present in a compound (e.g., a compound of Formula (I)) is called “deuterium-enrichment,” and such compounds are referred to as “deuterium-enriched” compounds. If not specifically noted, the percentage of enrichment refers to the percentage of deuterium present in the compound.

In other embodiments, a compound of this invention has an isotopic enrichment factor for each deuterium present at a site designated at a potential site of deuteration on the compound of at least 3500 (52.5.% deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6466.7 (97% deuterium incorporation), at least 6633.3 (99.5% deuterium incorporation). It is understood that the isotopic enrichment factor of each deuterium present at a site designated as a site of deuteration is independent of other deuterated sites. For example, if there are two sites of deuteration on a compound one site could be deuterated at 52.5% while the other could be deuterated at 75%. The resulting compound would be considered to be a compound wherein the isotopic enrichment factor is at least 3500 (52.5%).

Because the natural abundance of deuterium is about 0.015%, approximately one in every 6,667 naturally occurring sites of hydrogen in a compound described herein, e.g., a compound of Formula (I), would be expected to have a deuterium present.

All percentages given for the amount of deuterium present are mole percentages.

It can be difficult in the laboratory to achieve 100% deuteration at any one site of a lab scale amount of compound (e.g., milligram or greater). When 100% deuteration is recited or a deuterium atom is specifically shown in a structure, it is assumed that a small percentage of hydrogen may still be present. Deuterium-enriched can be achieved by either exchanging protons with deuterium or by synthesizing the molecule with enriched starting materials.

Also described herein is the isolation or purification of deuterium-enriched compounds of Formula (I). The isolated or purified deuterium-enriched compounds of Formula (I).

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The invention additionally encompasses compounds described herein as individual isomers substantially free of other isomers, and alternatively, as mixtures of various isomers.

Isomers, e.g., stereoisomers, can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The invention additionally encompasses compounds described herein as individual isomers substantially free of other isomers, and alternatively, as mixtures of various isomers.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

The term “diastereomerically pure” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of a single diastereomer. Methods for determining diastereomeric and enantiomeric purity are well-known in the art. Diastereomeric purity can be determined by any analytical method capable of quantitatively distinguishing between a compound and its diastereomers, such as high performance liquid chromatography (HPLC).

When a range of values is listed, it is intended to encompass each value and sub-range within the range. For example “C1-3 alkyl” is intended to encompass, C1, C2, C3, C1-3, C1-2, and C2-3.

The following terms are intended to have the meanings presented therewith below and are useful in understanding the description and intended scope of the present invention.

“Alkyl” refers to a radical of a straight-chain or branched saturated hydrocarbon group.

A “counterion” or “anionic counterion” is a negatively charged group associated with a cationic quaternary amino group in order to maintain electronic neutrality. Exemplary counterions include halide ions (e.g., F—, Cl—, Br—, I—), NO₃—, ClO₄—, OH—, H₂PO₄—, HSO₄—, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

These and other exemplary substituents are described in more detail in the Detailed Description, and Claims. The invention is not intended to be limited in any manner by the above exemplary listing of substituents.

### Other Definitions

As used herein, the term “positive modulation” or “positive allosteric modulation” refers to the potentiation of NMDA receptor function. A “positive modulator” or a “positive allosteric modulator” (e.g., a modulator compound) may be, for example, an agonist, partial agonist, of the NMDA receptor.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, et al., J. Pharm. Sci. (1977) 66(1): 1-79.

“Solvate” refers to forms of the compound that are associated with a solvent or water (also referred to as “hydrate”), usually by a solvolysis reaction. This physical association includes hydrogen bonding. Conventional solvents include water, ethanol, acetic acid, and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

“Stereoisomers”: It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

A “subject” to which administration is contemplated includes, but is not limited to, human subject (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

Disease, disorder, and condition are used interchangeably herein.

As used herein, the terms “treat,” “treating,” “treatment” or variations thereof includes reversing, reducing, or arresting the symptoms, clinical signs, and underlying pathology of a condition in manner to improve or stabilize a subject's condition. “Treatment” contemplates an action that occurs while a subject is suffering from the specified disease, disorder or condition, which reduces the severity of the disease, disorder or condition, or retards or slows the progression of the disease, disorder or condition. As used herein, and as well understood in the art, “treatment” is an approach for obtaining beneficial or desired results, including clinical results. Beneficial or desired clinical results can include, but are not limited to, alleviation, amelioration, reduction of the severity, or slowing the progression, of one or more symptoms or conditions associated with a condition, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Exemplary beneficial clinical results are described herein.

In general, the “effective amount” of a compound refers to an amount sufficient to elicit the desired biological response, e.g., to treat a CNS-related disorder, is sufficient to induce anesthesia or sedation. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject.

The terms “pharmaceutically effective amount,” “therapeutically effective amount,” or “therapeutically effective dose” refer to an amount sufficient to treat a disease in a patient, e.g., effecting a beneficial and/or desirable alteration in the health of a patient suffering from a disease, treatment, healing, inhibition or amelioration of a physiological response or condition, delaying or minimizing one or more symptoms associated with the disease, disorder or condition etc. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, the nature and extent of disease, the therapeutics or combination of therapeutics selected for administration, and the mode of administration. The skilled worker can readily determine the effective amount for a given situation by routine experimentation. The terms “pharmaceutically effective amount,” “therapeutically effective amount,” or “therapeutically effective dose” also refer to the amount required to improve the clinical symptoms of a patient. A therapeutically effective amount of a compound also refers to an amount of the therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

In an alternative embodiment, compounds of Formula (I) or a pharmaceutically acceptable salt or a pharmaceutically acceptable composition thereof, can also be administered in a “prophylactically effective amount”. As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

As used herein, and unless otherwise specified, “pharmacokinetics” can be defined as the study of bodily absorption, distribution, metabolism, and excretion of drugs. “Pharmacokinetics” can also be defined as the characteristic interactions of a drug and a body in terms of its absorption, distribution, metabolism, and excretion; or a branch of pharmacology concerned with the way drugs are taken into, move around, and are eliminated from, a body.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. In some embodiments, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. When a method is part of a therapeutic regimen involving more than one agent or treatment modality, the disclosure contemplates that the agents may be administered at the same or differing times and via the same or differing routes of administration. Appropriate methods of administering a substance, a compound or an agent to a subject will also depend, for example, on the age of the subject, whether the subject is active or inactive at the time of administering, whether the subject is cognitively impaired at the time of administering, the extent of the impairment, and the chemical and biological properties of the compound or agent (e.g. solubility, digestibility, bioavailability, stability and toxicity).

As generally described herein, the disclosure provides compounds useful for preventing and/or treating a broad range of disorders, including, but not limited to, NMDA-mediated disorders. These compounds are expected to show improved in vivo potency, pharmacokinetic (PK) properties, oral bioavailability, formulatability, stability, and/or safety as compared to other compounds.

### Compounds

In one aspect, the present disclosure provides a compound of Formula (I):

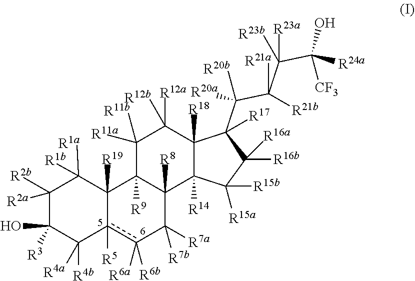

or a pharmaceutically acceptable salt thereof;
wherein:
each of R³, R¹⁸, R¹⁹, R^{20a} and R^{24a} is independently C₁₋₃alkyl optionally substituted with 1 or more independent occurrences of deuterium;
each of R^{1a}, R^{1b}, R^{2a}, R^{2b}, R^{4a}, R^{4b}, R⁵, R^{6a}, R^{6b}, R^{7a}, R^{7b}, R⁸, R⁹, R^{11a}, R^{11b}, R^{12a}, R^{12b}, R¹⁴, R^{15a}, R^{15b}, R^{16a}, R^{16b}, R¹⁷, R^{20b}, R^{21a}, R^{21b}, R^{23a}, and, R^{23b} is independently selected from a group consisting of hydrogen and deuterium;
provided that at least one of R³, R¹⁸, R¹⁹, R^{20a} and R^{24a} is substituted with 1 or more independent occurrences of deuterium, or at least one of R^{1a}, R^{1b}, R^{2a}, R^{2b}, R^{4a}, R^{4b}, R⁵, R^{6a}, R^{6b}, R^{7a}, R^{7b}, R⁸, R⁹, R^{11a}, R^{11b}, R^{12a}, R^{12b}, R¹⁴, R^{15a}, R^{15b}, R^{16a}, R^{16b}, R¹⁷, R^{20b}, R^{21a}, R^{21b}, R^{23a}, and R^{23b} is deuterium;
[symbol] represents a single or double bond; provided if the bond between C5 and C6 is a single bond, then R⁵ is in the alpha configuration.

In some embodiments, the compound of Formula (I) a compound of Formula (Ia):

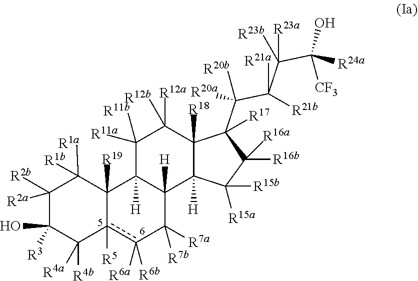

or a pharmaceutically acceptable salt thereof;
wherein:
each of R³, R¹⁸, R¹⁹, R^{20a} and R^{24a} is independently C₁₋₃alkyl optionally substituted with 1 or more independent occurrences of deuterium;
each of R^{1a}, R^{1b}, R^{2a}, R^{2b}, R^{4a}, R^{4b}, R⁵, R^{6a}, R^{6b}, R^{7a}, R^{7b}, R^{11a}, R^{11b}, R^{12a}, R^{12b}, R^{15a}, R^{15b}, R^{16a}, R^{16b}, R¹⁷, R^{20b}, R^{21a}, R^{21b}, R^{23a} and R^{23b} is independently selected from a group consisting of hydrogen and deuterium;
provided that at least one of R³, R¹⁸, R¹⁹, R^{20a} and R^{24a} is substituted with 1 or more independent occurrences of deuterium, or at least one of R^{1a}, R^{1b}, R^{2a}, R^{2b}, R^{4a}, R^{4b}, R⁵, R^{6a}, R^{6b}, R^{7a}, R^{7b}, R^{11a}, R^{11b}, R^{12a}, R^{12b}, R^{15a}, R^{15b}, R^{16a}, R^{16b}, R¹⁷, R^{20b}, R^{21a}, R^{21b}, R^{23a}, and R^{23b} is deuterium;
[symbol] represents a single or double bond; provided if the bond between C5 and C6 is a single bond, then R⁵ is in the alpha configuration.

In some embodiments, the compound of Formula (Ia) is a compound of Formula (II):

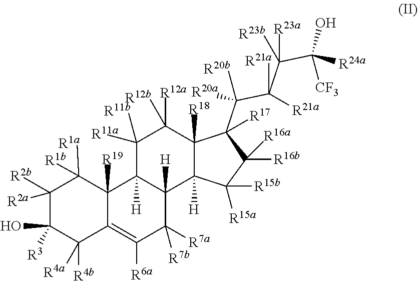

or a pharmaceutically acceptable salt thereof.

In some embodiments, the compound of Formula (Ia) is a compound of Formula (III):

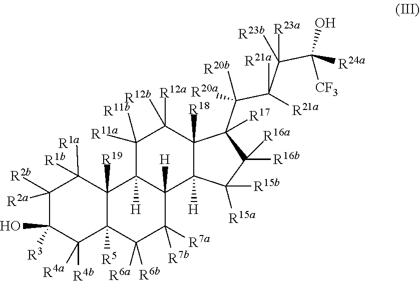

or a pharmaceutically acceptable salt thereof.

In some embodiments, the compound of Formula (I) is a compound of Formula (IV):

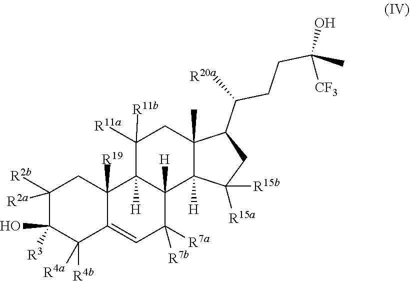

or a pharmaceutically acceptable salt thereof.

In some embodiments, the compound of formula ( ) is a compound of formula (V)

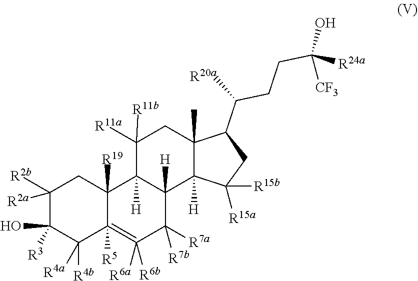

or a pharmaceutically acceptable salt thereof.

In some embodiments, the disclosure provides a compound as disclosed herein. In some embodiments, the disclosure provides a compound of Formula (I). In some embodiments, the disclosure provides a compound of Formula (Ia). In some embodiments, the disclosure provides a compound of Formula (II). In some embodiments, the disclosure provides a compound of Formula (III). In some embodiments, the disclosure provides a compound of Formula (IV). In some embodiments, the disclosure provides a compound of Formula (V).

In some embodiments, the disclosure provides a pharmaceutically acceptable salt of a compound disclosed herein. In some embodiments, the disclosure provides a pharmaceutically acceptable salt of a compound of Formula (I). In some embodiments, the disclosure provides a pharmaceutically acceptable salt of a compound of Formula (Ia). In some embodiments, the disclosure provides a pharmaceutically acceptable salt of a compound of Formula (II). In some embodiments, the disclosure provides a pharmaceutically acceptable salt of a compound of Formula (III). In some embodiments, the disclosure provides a pharmaceutically acceptable salt of a compound of Formula (IV). In some embodiments, the disclosure provides a pharmaceutically acceptable salt of a compound of Formula (V).

In some embodiments, each of R³, R¹⁸, R¹⁹, R^{20a} and R^{24a} is independently C₁₋₃alkyl optionally substituted with 1 or more independent occurrences of deuterium; and each of R^{1a}, R^{1b}, R^{2a}, R^{2b}, R^{4a}, R^{4b}, R⁵, R^{6a}, R^{6b}, R^{7a}, R^{7b}, R⁸, R⁹, R^{11a}, R^{11b}, R^{12a}, R^{12b}, R¹⁴, R^{15a}, R^{15b}, R^{16a}, R^{16b}, R¹⁷, R^{20b}, R^{21a}, R^{21b}, R^{23a}, and R^{23b} is independently selected from a group consisting of hydrogen and deuterium; provided that at least one of R³, R¹⁸, R¹⁹, R^{20a} and R^{24a} is substituted with 1 or more independent occurrences of deuterium, or at least one of R^{1a}, R^{1b}, R^{2a}, R^{2b}, R^{4a}, R^{4b}, R⁵, R^{6a}, R^{6b}, R^{7a}, R^{7b}, R⁸, R⁹, R^{11a}, R^{1b}, R^{12a}, R^{12b}, R¹⁴, R^{15a}, R^{15b}, R^{16a}, R^{16b}, R¹⁷, R^{20b}, R^{21a}, R^{21b}, R^{23a}, and R^{23b} is deuterium.

In some embodiments, each of R³, R¹⁸, R¹⁹, R^{20a} and R^{24a} is independently C₁₋₃alkyl optionally substituted with 1 or more independent occurrences of deuterium; and each of R^{1a}, R^{1b}, R^{2a}, R^{2b}, R^{4a}, R^{4b}, R⁵, R^{6a}, R^{6b}, R^{7a}, R^{7b}, R^{11a}, R^{11b}, R^{12a}, R^{12b}, R^{15a}, R^{15b}, R^{16a}, R^{16b}, R¹⁷, R^{20b}, R^{21a}, R^{21b}, R^{23a}, and R^{23b} is independently selected from a group consisting of hydrogen and deuterium; provided that at least one of R³, R¹⁸, R¹⁹, R^{20a} and R^{24a} is substituted with 1 or more independent occurrences of deuterium, or at least one of R^{1a}, R^{1b}, R^{2a}, R^{2b}, R^{4a}, R^{4b}, R⁵, R^{6a}, R^{6b}, R^{7a}, R^{7b}, R^{11a}, R^{11b}, R^{12a}, R^{12b}, R^{15a}, R^{15b}, R^{16a}, R^{16b}, R¹⁷, R^{20b}, R^{21a}, R^{21b}, R^{23a}, and R^{23b} is deuterium.

In certain embodiments, at least one of R^{3a}, R¹⁸, R¹⁹, R^{20a}, or R^{24a} is C₁₋₃alkyl substituted with one or more independent occurrences of deuterium, or at least one of R^{2a}, R^{2b}, R^{4a}, R^{4b}, R⁵, R^{6a}, R^{7a}, R^{7b}, R^{11a}, R^{11b}, R^{15a} and R^{15b} is deuterium. In some embodiments, at least one of R^{3a}, R¹⁸, R¹⁹, R^{20a}, or R^{24a} is C₁₋₃alkyl substituted with 1 or more independent occurrences of deuterium. In some embodiments, at least one of R^{2a}, R^{2b}, R^{4a}, R^{4b}, R⁵, R^{6a}, R^{7a}, R^{7b}, R^{11a}, R^{11b}, R^{15a} and R^{15b} is deuterium.

Groups R^{1a}, R^{1b}, R^{2a}, R^{2b}, R^{4a} and R^{4b}

In some embodiments, each of R^{1a} and R^{1b} is independently hydrogen or deuterium. In some embodiments, each of R^{1a} and R^{1b} is independently deuterium. In some embodiments, each of R^{1a} and R^{1b} is independently hydrogen.

In some embodiments, each of R^{2a} and R^{2b} is independently deuterium or hydrogen. In some embodiments, each of R^{2a} and R^{2b} is independently deuterium. In some embodiments, each of R^{2a} and R^{2b} is independently hydrogen.

In some embodiments, each of R^{4a} and R^{4b} is independently hydrogen or deuterium. In some embodiments, each of R^{4a} and R^{4b} is independently deuterium. In some embodiments, each of R^{4a} and R^{4b} is independently hydrogen.

### Groups R³

In some embodiments, R³ is C₁₋₃alkyl optionally substituted with 1 or more independent occurrences of deuterium. In some embodiments, R³ is C₁₋₃alkyl substituted with 1 or more independent occurrences of deuterium. In some embodiments, R³ is —CH₃. In some embodiments, R³ is methyl substituted with 1-3 independent occurrences of deuterium. In some embodiments, R³ is —CD₃.

Groups R⁵, R^{6a}, R^{6b}, R^{7a} and R^{7b}

In some embodiments, R⁵ is hydrogen or deuterium. In some embodiments, R⁵ is deuterium. In certain embodiments, R⁵ is hydrogen.

In some embodiments, each of R⁵ and R^{6a} is independently hydrogen or deuterium. In some embodiments, each of R⁵ and R^{6a} is independently deuterium. In some embodiments, each of R⁵ and R^{6a} is independently hydrogen.

In some embodiments, each of R^{6a} and R^{6b} is independently hydrogen or deuterium. In some embodiments, each of R^{6a} and R^{6b} is independently deuterium. In some embodiments, each of R^{6a} and R^{6b} is independently hydrogen.

In some embodiments, each of R^{7a} or R^{7b} is independently hydrogen or deuterium. In some embodiments, each of R^{7a} or R^{b} is independently deuterium. In some embodiments, each of R^{7a} or R^{7b} is independently hydrogen.

Groups, R⁸, R⁹, and R¹⁴

In some embodiments, R⁸ is hydrogen or deuterium. In some embodiments, R⁸ is deuterium. In some embodiments, R⁸ is hydrogen.

In some embodiments, R⁹ is hydrogen or deuterium. In some embodiments, R⁹ is deuterium. In some embodiments, R⁹ is hydrogen.

In some embodiments, R¹⁴ is hydrogen or deuterium. In some embodiments, R¹⁴ is deuterium. In some embodiments, R¹⁴ is hydrogen.

Groups R^{11a}, R^{11b}, R¹², and R^{12b}

In some embodiments, each of R^{11a} and R^{11b} is independently hydrogen or deuterium. In some embodiments, each of R^{11a} and R^{11b} is independently deuterium. In some embodiments, each of R^{11a} and R^{11b} is independently hydrogen. In some embodiments, each of R^{12a} and R^{12b} is independently hydrogen or deuterium. In some embodiments, each of R^{12a} and R^{12b} is independently deuterium. In some embodiments, each of R^{12a} and R^{12b} is independently hydrogen.

Groups R^{5a}, R^{5b}, R^{16a}, R^{6b}, and R¹⁷

In some embodiments, each of R^{15a} and R^{15b} is independently hydrogen or deuterium. In some embodiments, each of R^{15a} and R^{15b} is independently deuterium. In some embodiments, each of R^{15a} and R^{15b} is independently hydrogen.

In some embodiments, each of R^{16a} and R^{16b} is independently hydrogen or deuterium. In some embodiments, each of R^{16a} and R^{16b} is independently deuterium. In some embodiments, each of R^{16a} and R^{16b} is independently hydrogen.

In some embodiments, R¹⁷ is hydrogen or deuterium. In some embodiments, R¹⁷ is deuterium. In some embodiments, R¹⁷ is hydrogen.

Groups R⁸, and R¹⁹

In some embodiments, R¹⁸ is C₁₋₃alkyl optionally substituted with 1 or more independent occurrences of deuterium. In certain embodiments, R¹⁸ is C₁₋₃alkyl substituted with 1 or more independent occurrences of deuterium. In certain embodiments, R¹⁸ is methyl substituted with 1-3 independent occurrences of deuterium. In some embodiments, R¹⁸ is —CD₃. In some embodiments, R¹⁸ is —CH₃.

In some embodiments, R¹⁹ is C₁₋₃alkyl optionally substituted with 1 or more independent occurrences of deuterium. In some embodiments, R¹⁹ is C₁₋₃alkyl substituted with 1 or more independent occurrences of deuterium. In some embodiments, R¹⁹ is methyl substituted with 1-3 independent occurrences of deuterium. In some embodiments, R¹⁹ is —CD₃. In some embodiments, R¹⁹ is —CH₃.

Groups R^{20a}, and R^{20b}

In some embodiments, R^{20a} is C₁₋₃alkyl optionally substituted with 1 or more independent occurrences of deuterium. In some embodiments, R^{20a} is C₁₋₃alkyl substituted with 1 or more independent occurrences of deuterium. In some embodiments, R^{20a} is methyl substituted with 1-3 independent occurrences of deuterium. In some embodiments, R^{20a} is —CD₃. In some embodiments, R^{20a} is —CH₃.

In some embodiments, R^{20b} is deuterium. In some embodiments, R^{20b} is hydrogen.

Groups R^{21a}, R^{21b}, R^{23a}, R^{3b} and R^{24a}

In some embodiments, each of R^{21a} and R^{21b} is independently hydrogen or deuterium. In some embodiments, each of R^{21a} and R^{21b} is independently deuterium. In some embodiments, each of R^{21a} and R^{21b} is independently hydrogen.

In some embodiments, each of R^{23a} and R^{23b} is independently hydrogen or deuterium. In some embodiments, each of R^{23a} and R^{23b} is independently deuterium. In some embodiments, each of R^{23a} and R^{23b} is independently hydrogen.

In some embodiments, R^{24a} is C₁₋₃alkyl optionally is substituted with 1 or more independent occurrences of deuterium. In some embodiments, R^{24a} is C₁₋₃alkyl substituted with 1 or more independent occurrences of deuterium. In some embodiments, R^{24a} is methyl substituted with 1-3 independent occurrences of deuterium. In some embodiments, R^{24a} is —CD₃. In some embodiments, R^{24a} is —CH₃.

In some embodiments, the compound of Formula (IV) is selected from the group consisting of any one of compounds A-J:

| Compound | | | | | | | | | | | | | | |
|---|---|---|---|---|---|---|---|---|---|---|---|---|---|---|
| Number | R^{2a} | R^{2b} | R³ | R^{4a} | R^{4b} | R^{7a} | R^{7b} | R^{11a} | R^{11b} | R^{15a} | R^{15b} | R¹⁹ | R^{20a} | R^{24a} |
| A | D | D | CH₃ | H | H | H | H | H | H | H | H | CH₃ | CH₃ | CH₃ |
| B | H | H | CD₃ | H | H | H | H | H | H | H | H | CH₃ | CH₃ | CH₃ |
| C | H | H | —CH₃ | D | D | H | H | H | H | H | H | CH₃ | CH₃ | CH₃ |
| D | H | H | —CH₃ | H | H | D | D | H | H | H | H | CH₃ | CH₃ | CH₃ |
| E | H | H | —CH₃ | H | H | H | H | D | D | H | H | CH₃ | CH₃ | CH₃ |
| F | H | H | —CH₃ | H | H | H | H | H | H | D | D | CH₃ | CH₃ | CH₃ |
| G | H | H | —CH₃ | H | H | H | H | H | H | H | H | CD₃ | CH₃ | CH₃ |
| H | H | H | —CH₃ | H | H | H | H | H | H | H | H | CH₃ | CD₃ | CH₃ |
| I | H | H | —CH₃ | H | H | H | H | H | H | H | H | CH₃ | CH₃ | CD₃ |
| J | D | D | —CH₃ | D | D | H | H | H | H | H | H | CH₃ | CH₃ | CH₃ |

or a pharmaceutically acceptable salt thereof, wherein any atom not labeled as deuterium is present at its natural isotopic abundance. In some embodiments, the compound is a compound of Formula (IV) selected from the group consisting of any one of compounds A-J. In some embodiments, the compound is a pharmaceutically acceptable salt of a compound of Formula (IV) selected from the group consisting of any one of compounds A-J.

In some embodiments, the compound of Formula (V) is selected from the group consisting of any one of compounds K-X:

| Cmpd | | | | | | | | | | | | | | | | | |
|---|---|---|---|---|---|---|---|---|---|---|---|---|---|---|---|---|---|
| No | R^{2a} | R^{2b} | R³ | R^{4a} | R^{4b} | R⁵ | R^{6a} | R^{6b} | R^{7a} | R^{7b} | R^{11a} | R^{11b} | R^{15a} | R^{15b} | R¹⁹ | R^{20a} | R^{24a} |
| K | H | H | CH₃ | H | H | D | D | H | H | H | H | H | H | H | CH₃ | CH₃ | CH₃ |
| L | D | D | CH₃ | H | H | H | H | H | H | H | H | H | H | H | CH₃ | CH₃ | CH₃ |
| M | H | H | CD₃ | H | H | H | H | H | H | H | H | H | H | H | CH₃ | CH₃ | CH₃ |
| N | H | H | CH₃ | D | D | H | H | H | H | H | H | H | H | H | CH₃ | CH₃ | CH₃ |
| O | H | H | CH₃ | H | H | H | H | D | H | H | H | H | H | H | CH₃ | CH₃ | CH₃ |
| P | H | H | CH₃ | H | H | H | H | H | D | D | H | H | H | H | CH₃ | CH₃ | CH₃ |
| Q | H | H | CH₃ | H | H | H | H | H | H | H | D | D | H | H | CH₃ | CH₃ | CH₃ |
| R | H | H | CH₃ | H | H | H | H | H | H | H | H | H | D | D | CH₃ | CH₃ | CH₃ |
| S | H | H | CH₃ | H | H | H | H | H | H | H | H | H | H | H | CD₃ | CH₃ | CH₃ |
| T | H | H | CH₃ | H | H | H | H | H | H | H | H | H | H | H | CH₃ | CD₃ | CH₃ |
| U | H | H | CH₃ | H | H | H | H | H | H | H | H | H | H | H | CH₃ | CH₃ | CD₃ |
| V | H | H | CH₃ | H | H | D | H | H | H | H | H | H | H | H | CH₃ | CH₃ | CH₃ |
| W | H | H | CH₃ | H | H | H | D | D | H | H | H | H | H | H | CH₃ | CH₃ | CH₃ |
| X | D | D | CH₃ | D | D | H | H | H | H | H | H | H | H | H | CH₃ | CH₃ | CH₃ |

or a pharmaceutically acceptable salt thereof, wherein any atom not labeled as deuterium is present at its natural isotopic abundance. In some embodiments, the compound is a compound of Formula (V) selected from the group consisting of any one of compounds K-X. In some embodiments, the compound is a pharmaceutically acceptable salt of a compound of Formula (V) selected from the group consisting of any one of compounds K-X.

In some embodiments, the compound is selected from any one of the compounds in Table 1.

TABLE 1

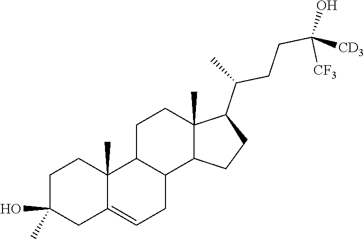

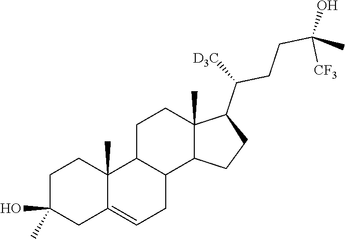

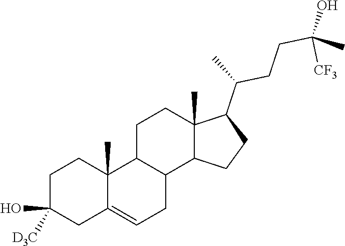

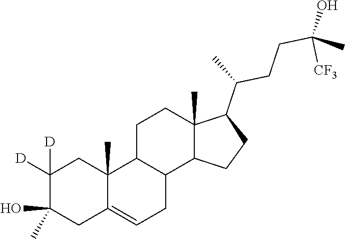

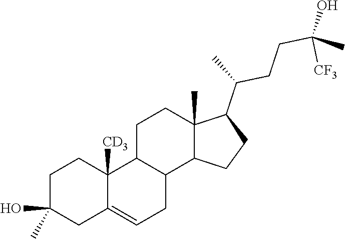

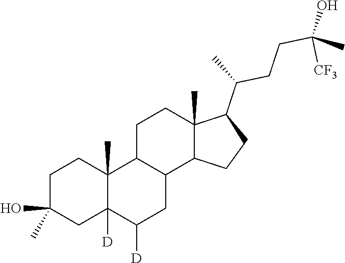

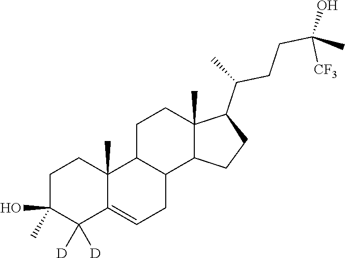

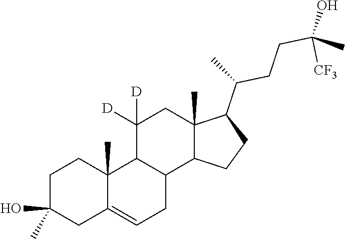

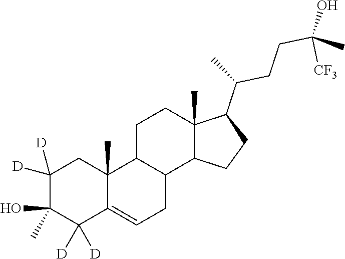

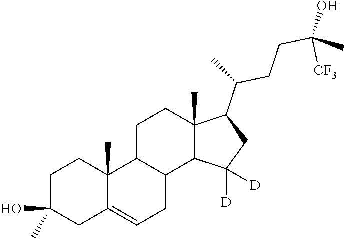

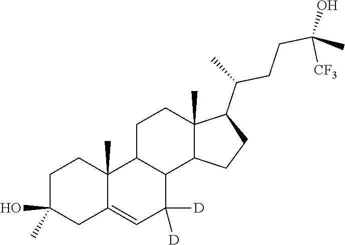

Exemplary compounds of the invention may be synthesized from the following known starting materials using methods known to one skilled in the art or certain references, e.g., U.S. Pat. No. 10,227,375, which is incorporated herein by reference:

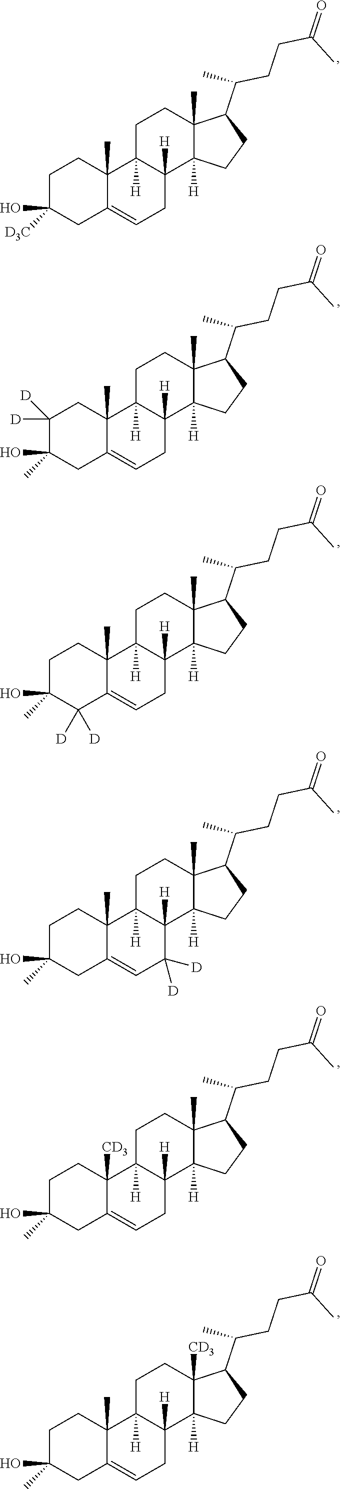

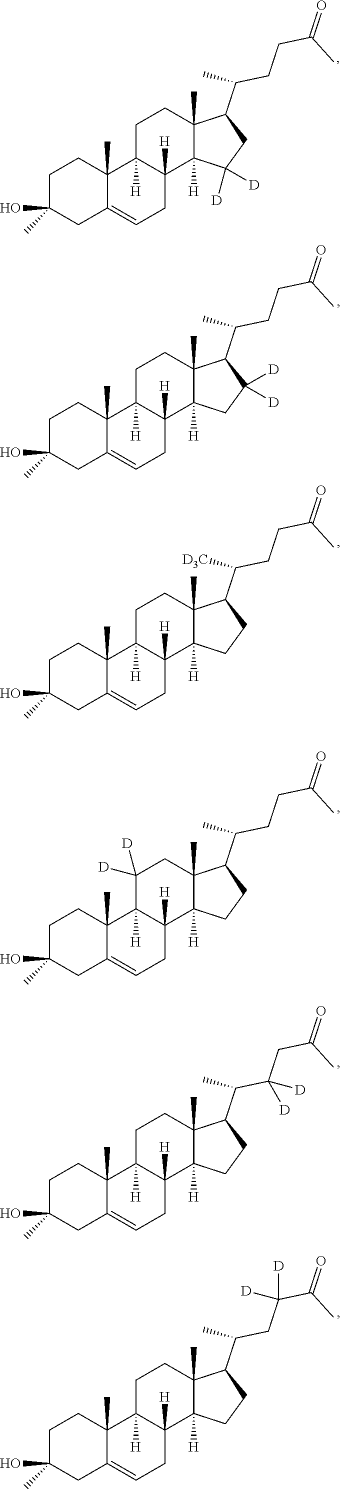

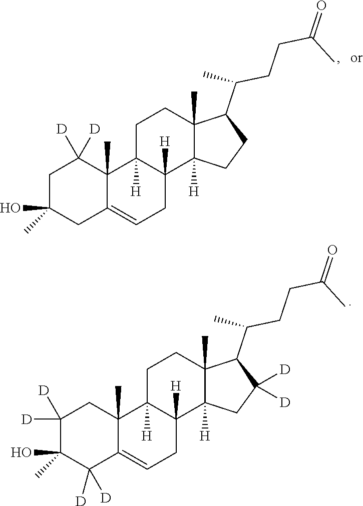

### Alternative Embodiments

In an alternative embodiment, compounds described herein may also comprise one or more isotopic substitutions of atoms other than hydrogen. For example, carbon may be, for example, ¹³C or ¹⁴C; oxygen may be, for example, ¹⁸O; nitrogen may be, for example, ¹⁵N, and the like. In other embodiments, a particular isotope (e.g., ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Pharmaceutical Compositions

In another aspect, the disclosure provides a pharmaceutical composition comprising a pharmaceutically acceptable carrier and an effective amount of a compound described herein (e.g., a compound of Formula (I)) or a pharmaceutically acceptable salt thereof, and a pharmaceutically acceptable excipient. In certain embodiments, the disclosure provides a pharmaceutical composition comprising a pharmaceutically acceptable carrier and an effective amount of a compound described herein (e.g., a compound of Formula (I)), and a pharmaceutically acceptable excipient. In certain embodiments, the compound of the present invention is provided in an effective amount in the pharmaceutical composition. In certain embodiments, the compound of the present invention is provided in a therapeutically effective amount.

In certain embodiments, the pharmaceutical composition comprises an effective amount of the active ingredient. In certain embodiments, the pharmaceutical composition comprises a therapeutically effective amount of the active ingredient.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration.

The present disclosure also relates to a compound described herein (e.g., a compound of Formula (I), or pharmaceutical composition thereof) for use as a pharmaceutical or a medicament.

Generally, the compounds provided herein are administered in a therapeutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

When used to prevent the onset of a CNS-disorder, the compounds provided herein will be administered to a subject at risk for developing the condition, typically on the advice and under the supervision of a physician, at the dosage levels described above. Subjects at risk for developing a particular condition generally include those that have a family history of the condition, or those who have been identified by genetic testing or screening to be particularly susceptible to developing the condition.

The pharmaceutical compositions provided herein can also be administered chronically (“chronic administration”). Chronic administration refers to administration of a compound or pharmaceutical composition thereof over an extended period of time, e.g., for example, over 3 months, 6 months, 1 year, 2 years, 3 years, 5 years, etc, or may be continued indefinitely, for example, for the rest of the subject's life. In certain embodiments, the chronic administration is intended to provide a constant level of the compound in the blood, e.g., within the therapeutic window over the extended period of time.

The pharmaceutical compositions of the present invention may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or excipients and processing aids helpful for forming the desired dosing form.

With oral dosing, one to five and especially two to four and typically three oral doses per day are representative regimens. Using these dosing patterns, each dose provides from about 0.01 to about 20 mg/kg of the compound provided herein, with preferred doses each providing from about 0.1 to about 10 mg/kg, and especially about 1 to about 5 mg/kg.

Transdermal doses are generally selected to provide similar or lower blood levels than are achieved using injection doses, generally in an amount ranging from about 0.01 to about 20% by weight, preferably from about 0.1 to about 20% by weight, preferably from about 0.1 to about 10% by weight, and more preferably from about 0.5 to about 15% by weight.

Injection dose levels range from about 0.1 mg/kg/hour to at least 20 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 5 g/day for a 40 to 80 kg human patient.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable excipients known in the art. As before, the active compound in such compositions is typically a minor component, often being from about 0.05 to 10% by weight with the remainder being the injectable excipient and the like.

Transdermal compositions are typically formulated as a topical ointment or cream containing the active ingredient(s). When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or Formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The compounds of the present invention can also be administered in sustained release forms or from sustained release drug delivery systems. A description of representative sustained release materials can be found in Remington's Pharmaceutical Sciences.

The present invention also relates to the pharmaceutically acceptable acid addition salt of a compound of the present invention. The acid which may be used to prepare the pharmaceutically acceptable salt is that which forms a non-toxic acid addition salt, i.e., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

In another aspect, the invention provides a pharmaceutical composition comprising a compound of the present invention and a pharmaceutically acceptable excipient, e.g., a composition suitable for injection, such as for intravenous (IV) administration.

Pharmaceutically acceptable excipients include any and all diluents or other liquid vehicles, dispersion or suspension aids, surface active agents, isotonic agents, preservatives, lubricants and the like, as suited to the particular dosage form desired, e.g., injection. General considerations in the formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

For example, injectable preparations, such as sterile injectable aqueous suspensions, can be formulated according to the known art using suitable dispersing or wetting agents and suspending agents. Exemplary excipients that can be employed include, but are not limited to, water, sterile saline or phosphate-buffered saline, or Ringer's solution.

In certain embodiments, the pharmaceutical composition further comprises a cyclodextrin derivative. The most common cyclodextrins are α-, β- and γ-cyclodextrins consisting of 6, 7 and 8 α-1,4-linked glucose units, respectively, optionally comprising one or more substituents on the linked sugar moieties, which include, but are not limited to, substituted or unsubstituted methylated, hydroxyalkylated, acylated, and sulfoalkylether substitution. In certain embodiments, the cyclodextrin is a sulfoalkyl ether β-cyclodextrin, e.g., for example, sulfobutyl ether β-cyclodextrin, also known as CAPTISOL®. See, e.g., U.S. Pat. No. 5,376,645. In certain embodiments, the composition comprises hexapropyl-β-cyclodextrin. In a more particular embodiment, the composition comprises hexapropyl-β-cyclodextrin (10-50% in water).

The injectable composition can be sterilized, for example, by filtration through a bacterial-retaining filter, or by incorporating sterilizing agents in the form of sterile solid compositions which can be dissolved or dispersed in sterile water or other sterile injectable medium prior to use.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, response of the individual patient, the severity of the patient's symptoms, and the like.

The compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include pre-filled, pre-measured ampules or syringes of the liquid compositions. In such compositions, the compound is usually a minor component (from about 0.1% to about 50% by weight or preferably from about 1% to about 40% by weight) with the remainder being various vehicles or carriers and processing aids helpful for forming the desired dosing form.

The compounds provided herein can be administered as the sole active agent, or they can be administered in combination with other active agents. In one aspect, the present invention provides a combination of a compound of the present invention and another pharmacologically active agent. Administration in combination can proceed by any technique apparent to those of skill in the art including, for example, separate, sequential, concurrent, and alternating administration.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21st ed., Lippincott Williams & Wilkins, 2005.

In one aspect, provided is a kit comprising a composition (e.g., a solid composition) comprising a compound of Formula (I).

In one aspect, the disclosure provides a pharmaceutical composition comprising: (a) a compound disclosed herein; (b) an anti-amyloid beta antibody or antigen-binding fragment thereof; and (c) a pharmaceutically acceptable carrier.

In some embodiments, the anti-amyloid beta antibody or antigen-binding fragment thereof is selected from the group consisting of bapineuzumab, solanezumab, gantenerumab, crenezumab, ponezumab, lecanemab (BAN2401), aducanumab, and antigen-binding fragments thereof. In some embodiments, the anti-amyloid beta antibody or antigen-binding fragment thereof is bapineuzumab or an antigen-binding fragments thereof. In some embodiments, the anti-amyloid beta antibody or antigen-binding fragment thereof is solanezumab, or an antigen-binding fragments thereof. In some embodiments, the anti-amyloid beta antibody or antigen-binding fragment thereof is gantenerumab or an antigen-binding fragments thereof. In some embodiments, the anti-amyloid beta antibody or antigen-binding fragment thereof is crenezumab or an antigen-binding fragments thereof. In some embodiments, the anti-amyloid beta antibody or antigen-binding fragment thereof is ponezumab or an antigen-binding fragments thereof. In some embodiments, the anti-amyloid beta antibody or antigen-binding fragment thereof is lecanemab (BAN2401) or an antigen-binding fragments thereof. In some embodiments, the anti-amyloid beta antibody or antigen-binding fragment thereof is aducanumab or an antigen-binding fragments thereof.

In some embodiments, the anti-amyloid beta antibody or antigen binding fragment thereof binds to the same conformational epitope of amyloid beta to which any one of bapineuzumab, solanezumab, gantenerumab, crenezumab, ponezumab, lecanemab (BAN2401), or aducanumab binds. In some embodiments, the anti-amyloid beta antibody or antigen binding fragment thereof binds to the same conformational epitope of amyloid beta to which bapineuzumab binds. In some embodiments, the anti-amyloid beta antibody or antigen binding fragment thereof binds to the same conformational epitope of amyloid beta to which solanezumab binds. In some embodiments, the anti-amyloid beta antibody or antigen binding fragment thereof binds to the same conformational epitope of amyloid beta to which gantenerumab binds. In some embodiments, the anti-amyloid beta antibody or antigen binding fragment thereof binds to the same conformational epitope of amyloid beta to which crenezumab binds. In some embodiments, the anti-amyloid beta antibody or antigen binding fragment thereof binds to the same conformational epitope of amyloid beta to which ponezumab binds. In some embodiments, the anti-amyloid beta antibody or antigen binding fragment thereof binds to the same conformational epitope of amyloid beta to which lecanemab (BAN2401) binds. In some embodiments, the anti-amyloid beta antibody or antigen binding fragment thereof binds to the same conformational epitope of amyloid beta to which aducanumab binds.

In some embodiments, the anti-amyloid beta antibody or antigen binding fragment thereof competitively inhibits binding of any one of bapineuzumab, solanezumab, gantenerumab, crenezumab, ponezumab, lecanemab (BAN2401), or aducanumab to amyloid beta. In some embodiments, the anti-amyloid beta antibody or antigen binding fragment thereof competitively inhibits binding of bapineuzumab to amyloid beta. In some embodiments, the anti-amyloid beta antibody or antigen binding fragment thereof competitively inhibits binding of solanezumab to amyloid beta. In some embodiments, the anti-amyloid beta antibody or antigen binding fragment thereof competitively inhibits binding of gantenerumab to amyloid beta. In some embodiments, the anti-amyloid beta antibody or antigen binding fragment thereof competitively inhibits binding of ponezumab to amyloid beta. In some embodiments, the anti-amyloid beta antibody or antigen binding fragment thereof competitively inhibits binding of lecanemab (BAN2401) to amyloid beta. In some embodiments, the anti-amyloid beta antibody or antigen binding fragment thereof competitively inhibits binding of aducanumab to amyloid beta.

In some embodiments, the anti-amyloid beta antibody or antigen-binding fragment thereof comprises the 6 CDRs of any one of the anti-amyloid beta antibodies disclosed herein as determined by Kabat, Chothia or IMTG nomenclature. In some embodiments, the anti-amyloid beta antibody or antigen-binding fragment thereof comprises the 6 CDRs of any one of bapineuzumab, solanezumab, gantenerumab, crenezumab, ponezumab, lecanemab (BAN2401), aducanumab. In some embodiments, the anti-amyloid beta antibody or antigen-binding fragment thereof comprises the 6 CDRs of bapineuzumab. In some embodiments, the anti-amyloid beta antibody or antigen-binding fragment thereof comprises the 6 CDRs of solanezumab. In some embodiments, the anti-amyloid beta antibody or antigen-binding fragment thereof comprises the 6 CDRs of gantenerumab. In some embodiments, the anti-amyloid beta antibody or antigen-binding fragment thereof comprises the 6 CDRs of crenezumab. In some embodiments, the anti-amyloid beta antibody or antigen-binding fragment thereof comprises the 6 CDRs of ponezumab. In some embodiments, the anti-amyloid beta antibody or antigen-binding fragment thereof comprises the 6 CDRs of lecanemab (BAN2401). In some embodiments, the anti-amyloid beta antibody or antigen-binding fragment thereof comprises the 6 CDRs of aducanumab.

In some embodiments, the anti-amyloid beta antibody or antigen-binding fragment thereof comprises:
(a) a heavy chain variable domain (VH) comprising (i) HCDR1 comprising the amino acid sequence of SEQ ID NO: 1, (ii) HCDR2 comprising the amino acid sequence of SEQ ID NO: 2, and (iii) HCDR3 comprising the amino acid sequence of SEQ ID NO: 3; and a light chain variable domain (VL) comprising (i) LCDR1 comprising the amino acid sequence of SEQ ID NO: 4, (ii) LCDR2 comprising the amino acid sequence of SEQ ID NO: 5, and (iii) LCDR3 comprising the amino acid sequence of SEQ ID NO: 6;
(b) a heavy chain variable domain (VH) comprising (i) HCDR1 comprising the amino acid sequence of SEQ ID NO: 11, (ii) HCDR2 comprising the amino acid sequence of SEQ ID NO: 12, and (iii) HCDR3 comprising the amino acid sequence of SEQ ID NO: 13; and a light chain variable domain (VL) comprising (i) LCDR1 comprising the amino acid sequence of SEQ ID NO: 14, (ii) LCDR2 comprising the amino acid sequence of SEQ ID NO: 15, and (iii) LCDR3 comprising the amino acid sequence of SEQ ID NO: 16;
(c) a heavy chain variable domain (VH) comprising (i) HCDR1 comprising the amino acid sequence of SEQ ID NO: 21, (ii) HCDR2 comprising the amino acid sequence of SEQ ID NO: 22, and (iii) HCDR3 comprising the amino acid sequence of SEQ ID NO: 23; and a light chain variable domain (VL) comprising (i) LCDR1 comprising the amino acid sequence of SEQ ID NO: 24, (ii) LCDR2 comprising the amino acid sequence of SEQ ID NO: 25, and (iii) LCDR3 comprising the amino acid sequence of SEQ ID NO: 26;
(d) a heavy chain variable domain (VH) comprising (i) HCDR1 comprising the amino acid sequence of SEQ ID NO: 31, (ii) HCDR2 comprising the amino acid sequence of SEQ ID NO: 32, and (iii) HCDR3 comprising the amino acid sequence of SEQ ID NO: 33; and a light chain variable domain (VL) comprising (i) LCDR1 comprising the amino acid sequence of SEQ ID NO: 34, (ii) LCDR2 comprising the amino acid sequence of SEQ ID NO: 35, and (iii) LCDR3 comprising the amino acid sequence of SEQ ID NO: 36;
(e) a heavy chain variable domain (VH) comprising (i) HCDR1 comprising the amino acid sequence of SEQ ID NO: 41, (ii) HCDR2 comprising the amino acid sequence of SEQ ID NO: 42, and (iii) HCDR3 comprising the amino acid sequence of SEQ ID NO: 43; and a light chain variable domain (VL) comprising (i) LCDR1 comprising the amino acid sequence of SEQ ID NO: 44, (ii) LCDR2 comprising the amino acid sequence of SEQ ID NO: 45, and (iii) LCDR3 comprising the amino acid sequence of SEQ ID NO: 46;
(f) a heavy chain variable domain (VH) comprising (i) HCDR1 comprising the amino acid sequence of SEQ ID NO: 51, (ii) HCDR2 comprising the amino acid sequence of SEQ ID NO: 52, and (iii) HCDR3 comprising the amino acid sequence of SEQ ID NO: 53; and a light chain variable domain (VL) comprising (i) LCDR1 comprising the amino acid sequence of SEQ ID NO: 54, (ii) LCDR2 comprising the amino acid sequence of SEQ ID NO: 55, and (iii) LCDR3 comprising the amino acid sequence of SEQ ID NO: 56; or
(g) a heavy chain variable domain (VH) comprising (i) HCDR1 comprising the amino acid sequence of SEQ ID NO: 61, (ii) HCDR2 comprising the amino acid sequence of SEQ ID NO: 62, and (iii) HCDR3 comprising the amino acid sequence of SEQ ID NO: 63; and a light chain variable domain (VL) comprising (i) LCDR1 comprising the amino acid sequence of SEQ ID NO: 64, (ii) LCDR2 comprising the amino acid sequence of SEQ ID NO: 65, and (iii) LCDR3 comprising the amino acid sequence of SEQ ID NO: 66.

In some embodiments, the anti-amyloid beta antibody or antigen-binding fragment thereof comprises: (a) a heavy chain variable domain (VH) comprising (i) HCDR1 comprising the amino acid sequence of SEQ ID NO: 1, (ii) HCDR2 comprising the amino acid sequence of SEQ ID NO: 2, and (iii) HCDR3 comprising the amino acid sequence of SEQ ID NO: 3; and (b) a light chain variable domain (VL) comprising (i) LCDR1 comprising the amino acid sequence of SEQ ID NO: 4, (ii) LCDR2 comprising the amino acid sequence of SEQ ID NO: 5, and (iii) LCDR3 comprising the amino acid sequence of SEQ ID NO: 6.

In some embodiments, the anti-amyloid beta antibody or antigen-binding fragment thereof comprises: (a) a heavy chain variable domain (VH) comprising (i) HCDR1 comprising the amino acid sequence of SEQ ID NO: 11, (ii) HCDR2 comprising the amino acid sequence of SEQ ID NO: 12, and (iii) HCDR3 comprising the amino acid sequence of SEQ ID NO: 13; and (b) a light chain variable domain (VL) comprising (i) LCDR1 comprising the amino acid sequence of SEQ ID NO: 14, (ii) LCDR2 comprising the amino acid sequence of SEQ ID NO: 15, and (iii) LCDR3 comprising the amino acid sequence of SEQ ID NO: 16.

In some embodiments, the anti-amyloid beta antibody or antigen-binding fragment thereof comprises: (a) a heavy chain variable domain (VH) comprising (i) HCDR1 comprising the amino acid sequence of SEQ ID NO: 21, (ii) HCDR2 comprising the amino acid sequence of SEQ ID NO: 22, and (iii) HCDR3 comprising the amino acid sequence of SEQ ID NO: 23; and (b) a light chain variable domain (VL) comprising (i) LCDR1 comprising the amino acid sequence of SEQ ID NO: 24, (ii) LCDR2 comprising the amino acid sequence of SEQ ID NO: 25, and (iii) LCDR3 comprising the amino acid sequence of SEQ ID NO: 26.

In some embodiments, the anti-amyloid beta antibody or antigen-binding fragment thereof comprises: (a) a heavy chain variable domain (VH) comprising (i) HCDR1 comprising the amino acid sequence of SEQ ID NO: 31, (ii) HCDR2 comprising the amino acid sequence of SEQ ID NO: 32, and (iii) HCDR3 comprising the amino acid sequence of SEQ ID NO: 33; and (b) a light chain variable domain (VL) comprising (i) LCDR1 comprising the amino acid sequence of SEQ ID NO: 34, (ii) LCDR2 comprising the amino acid sequence of SEQ ID NO: 35, and (iii) LCDR3 comprising the amino acid sequence of SEQ ID NO: 36.

In some embodiments, the anti-amyloid beta antibody or antigen-binding fragment thereof comprises: (a) a heavy chain variable domain (VH) comprising (i) HCDR1 comprising the amino acid sequence of SEQ ID NO: 41, (ii) HCDR2 comprising the amino acid sequence of SEQ ID NO: 42, and (iii) HCDR3 comprising the amino acid sequence of SEQ ID NO: 43; and (b) a light chain variable domain (VL) comprising (i) LCDR1 comprising the amino acid sequence of SEQ ID NO: 44, (ii) LCDR2 comprising the amino acid sequence of SEQ ID NO: 45, and (iii) LCDR3 comprising the amino acid sequence of SEQ ID NO: 46.

In some embodiments, the anti-amyloid beta antibody or antigen-binding fragment thereof comprises: (a) a heavy chain variable domain (VH) comprising (i) HCDR1 comprising the amino acid sequence of SEQ ID NO: 51, (ii) HCDR2 comprising the amino acid sequence of SEQ ID NO: 52, and (iii) HCDR3 comprising the amino acid sequence of SEQ ID NO: 53; and (b) a light chain variable domain (VL) comprising (i) LCDR1 comprising the amino acid sequence of SEQ ID NO: 54, (ii) LCDR2 comprising the amino acid sequence of SEQ ID NO: 55, and (iii) LCDR3 comprising the amino acid sequence of SEQ ID NO: 56.

In some embodiments, the anti-amyloid beta antibody or antigen-binding fragment thereof comprises: (a) a heavy chain variable domain (VH) comprising (i) HCDR1 comprising the amino acid sequence of SEQ ID NO: 61, (ii) HCDR2 comprising the amino acid sequence of SEQ ID NO: 62, and (iii) HCDR3 comprising the amino acid sequence of SEQ ID NO: 63; and (b) a light chain variable domain (VL) comprising (i) LCDR1 comprising the amino acid sequence of SEQ ID NO: 64, (ii) LCDR2 comprising the amino acid sequence of SEQ ID NO: 65, and (iii) LCDR3 comprising the amino acid sequence of SEQ ID NO: 66.

In some embodiments, the anti-amyloid beta antibody or antigen-binding fragment thereof comprises the heavy chain variable domain (VH) and/or the light chain variable domain (VL) domain of any one of the anti-amyloid beta antibodies disclosed herein. In some embodiments, the anti-amyloid beta antibody or antigen-binding fragment thereof comprises the VH domain of any one of bapineuzumab, solanezumab, gantenerumab, crenezumab, ponezumab, lecanemab (BAN2401), aducanumab.

In some embodiments, the anti-amyloid beta antibody or antigen-binding fragment thereof comprises the VH domain of any one of bapineuzumab, solanezumab, gantenerumab, crenezumab, ponezumab, lecanemab (BAN2401), aducanumab. In some embodiments, the anti-amyloid beta antibody or antigen-binding fragment thereof comprises the VH domain of bapineuzumab. In some embodiments, the anti-amyloid beta antibody or antigen-binding fragment thereof comprises the VH domain of solanezumab. In some embodiments, the anti-amyloid beta antibody or antigen-binding fragment thereof comprises the VH domain of gantenerumab. In some embodiments, the anti-amyloid beta antibody or antigen-binding fragment thereof comprises the VH domain of crenezumab. In some embodiments, the anti-amyloid beta antibody or antigen-binding fragment thereof comprises the VH domain of ponezumab. In some embodiments, the anti-amyloid beta antibody or antigen-binding fragment thereof comprises the VH domain of lecanemab (BAN2401). In some embodiments, the anti-amyloid beta antibody or antigen-binding fragment thereof comprises the VH domain of aducanumab.

In some embodiments, the anti-amyloid beta antibody or antigen-binding fragment thereof comprises the VL domain of any one of bapineuzumab, solanezumab, gantenerumab, crenezumab, ponezumab, lecanemab (BAN2401), aducanumab. In some embodiments, the anti-amyloid beta antibody or antigen-binding fragment thereof comprises the VL domain of bapineuzumab. In some embodiments, the anti-amyloid beta antibody or antigen-binding fragment thereof comprises the VL domain of solanezumab. In some embodiments, the anti-amyloid beta antibody or antigen-binding fragment thereof comprises the VL domain of gantenerumab. In some embodiments, the anti-amyloid beta antibody or antigen-binding fragment thereof comprises the VL domain of crenezumab. In some embodiments, the anti-amyloid beta antibody or antigen-binding fragment thereof comprises the VL domain of ponezumab. In some embodiments, the anti-amyloid beta antibody or antigen-binding fragment thereof comprises the VL domain of lecanemab (BAN2401). In some embodiments, the anti-amyloid beta antibody or antigen-binding fragment thereof comprises the VL domain of aducanumab.

In some embodiments, the anti-amyloid beta antibody or antigen-binding fragment thereof comprises the VH and VH domains of any one of bapineuzumab, solanezumab, gantenerumab, crenezumab, ponezumab, lecanemab (BAN2401), aducanumab. In some embodiments, the anti-amyloid beta antibody or antigen-binding fragment thereof comprises the VH and VL domains of bapineuzumab. In some embodiments, the anti-amyloid beta antibody or antigen-binding fragment thereof comprises the VH and VL domains of solanezumab. In some embodiments, the anti-amyloid beta antibody or antigen-binding fragment thereof comprises the VH and VL domains of gantenerumab. In some embodiments, the anti-amyloid beta antibody or antigen-binding fragment thereof comprises the VH and VL domains of crenezumab. In some embodiments, the anti-amyloid beta antibody or antigen-binding fragment thereof comprises the VH and VL domains of ponezumab. In some embodiments, the anti-amyloid beta antibody or antigen-binding fragment thereof comprises the VH and VL domains of lecanemab (BAN2401). In some embodiments, the anti-amyloid beta antibody or antigen-binding fragment thereof comprises the VH and VL domains of aducanumab.

In some embodiments, the anti-amyloid beta antibody or antigen-binding fragment thereof comprises a heavy chain variable domain (VH) comprising: (a) the amino acid sequence of SEQ ID NO:7; (b) the amino acid sequence of SEQ ID NO:17; (c) the amino acid sequence of SEQ ID NO:27; (d) the amino acid sequence of SEQ ID NO:37; (e) the amino acid sequence of SEQ ID NO:47; (f) the amino acid sequence of SEQ ID NO:57; or (g) the amino acid sequence of SEQ ID NO:67.

In some embodiments, the anti-amyloid beta antibody or antigen-binding fragment thereof comprises a light chain variable domain (VL) comprising: (a) the amino acid sequence of SEQ ID NO:8; (b) the amino acid sequence of SEQ ID NO:18; (c) the amino acid sequence of SEQ ID NO:28; (d) the amino acid sequence of SEQ ID NO:38; (e) the amino acid sequence of SEQ ID NO:48; (f) the amino acid sequence of SEQ ID NO:58; or (g) the amino acid sequence of SEQ ID NO:68.

In some embodiments, the anti-amyloid beta antibody or antigen-binding fragment thereof comprises a heavy chain variable domain (VH) comprising the amino acid sequence of SEQ ID NO:7.

In some embodiments, the anti-amyloid beta antibody or antigen-binding fragment thereof comprises a light chain variable domain (VL) comprising the amino acid sequence of SEQ ID NO:8.

In some embodiments, the anti-amyloid beta antibody or antigen-binding fragment thereof comprises a the heavy chain variable domain (VH) comprising the amino acid sequence of SEQ ID NO:7; and a light chain variable domain (VL) comprising the amino acid sequence of SEQ ID NO:8.

In some embodiments, the anti-amyloid beta antibody or antigen-binding fragment thereof comprises a heavy chain variable domain (VH) comprising the amino acid sequence of SEQ ID NO: 17. In some embodiments, the anti-amyloid beta antibody or antigen-binding fragment thereof comprises a light chain variable domain (VL) comprising the amino acid sequence of SEQ ID NO: 18. In some embodiments, the anti-amyloid beta antibody or antigen-binding fragment thereof comprises a heavy chain variable domain (VH) comprising the amino acid sequence of SEQ ID NO: 17; and a light chain variable domain (VL) comprising the amino acid sequence of SEQ ID NO:18.

In some embodiments, the anti-amyloid beta antibody or antigen-binding fragment thereof comprises a heavy chain variable domain (VH) comprising the amino acid sequence of SEQ ID NO: 27. In some embodiments, the anti-amyloid beta antibody or antigen-binding fragment thereof comprises a light chain variable domain (VL) comprising the amino acid sequence of SEQ ID NO: 28. In some embodiments, the anti-amyloid beta antibody or antigen-binding fragment thereof comprises a heavy chain variable domain (VH) comprising the amino acid sequence of SEQ ID NO:27; and a light chain variable domain (VL) comprising the amino acid sequence of SEQ ID NO:28.

In some embodiments, the anti-amyloid beta antibody or antigen-binding fragment thereof comprises a heavy chain variable domain (VH) comprising the amino acid sequence of SEQ ID NO: 37. In some embodiments, the anti-amyloid beta antibody or antigen-binding fragment thereof comprises a light chain variable domain (VL) comprising the amino acid sequence of SEQ ID NO: 38. In some embodiments, the anti-amyloid beta antibody or antigen-binding fragment thereof comprises a heavy chain variable domain (VH) comprising the amino acid sequence of SEQ ID NO:37; and a light chain variable domain (VL) comprising the amino acid sequence of SEQ ID NO:38.

In some embodiments, the anti-amyloid beta antibody or antigen-binding fragment thereof comprises a heavy chain variable domain (VH) comprising the amino acid sequence of SEQ ID NO: 47. In some embodiments, the anti-amyloid beta antibody or antigen-binding fragment thereof comprises a light chain variable domain (VL) comprising the amino acid sequence of SEQ ID NO: 48. In some embodiments, the anti-amyloid beta antibody or antigen-binding fragment thereof comprises a heavy chain variable domain (VH) comprising the amino acid sequence of SEQ ID NO:47; and a light chain variable domain (VL) comprising the amino acid sequence of SEQ ID NO:48.

In some embodiments, the anti-amyloid beta antibody or antigen-binding fragment thereof comprises a heavy chain variable domain (VH) comprising the amino acid sequence of SEQ ID NO: 57. In some embodiments, the anti-amyloid beta antibody or antigen-binding fragment thereof comprises a light chain variable domain (VL) comprising the amino acid sequence of SEQ ID NO: 58. In some embodiments, the anti-amyloid beta antibody or antigen-binding fragment thereof comprises a heavy chain variable domain (VH) comprising the amino acid sequence of SEQ ID NO:57; and a light chain variable domain (VL) comprising the amino acid sequence of SEQ ID NO:58.

In some embodiments, the anti-amyloid beta antibody or antigen-binding fragment thereof comprises a heavy chain variable domain (VH) comprising the amino acid sequence of SEQ ID NO: 67. In some embodiments, the anti-amyloid beta antibody or antigen-binding fragment thereof comprises a light chain variable domain (VL) comprising the amino acid sequence of SEQ ID NO: 68. In some embodiments, the anti-amyloid beta antibody or antigen-binding fragment thereof comprises a heavy chain variable domain (VH) comprising the amino acid sequence of SEQ ID NO:67; and a light chain variable domain (VL) comprising the amino acid sequence of SEQ ID NO:68.

In some embodiments, the anti-amyloid beta antibody or antigen-binding fragment thereof comprises a human IgG1, IgG2, IgG3, or IgG4 constant domain. In some embodiments, the anti-amyloid beta antibody or antigen-binding fragment thereof comprises a human IgG1 constant region.

In some embodiments, the anti-amyloid beta antibody or antigen-binding fragment thereof is a human anti-amyloid beta antibody or antigen-binding fragment thereof.

In some embodiments, the antigen-binding fragment is a single chain antibody, Fv, Fab, Fab′, F(ab′)₂, Fd, single chain Fv molecule (scFv), bispecific single chain Fv dimer, diabody, domain-deleted antibody or single domain antibody (dAb).

In some embodiments, the anti-amyloid beta antibody or antigen-binding fragment thereof comprises the heavy chain and/or the light chain of any one of the anti-amyloid beta antibodies disclosed herein.

In some embodiments, the anti-amyloid beta antibody or antigen-binding fragment thereof comprises the heavy chain of any one of bapineuzumab, solanezumab, gantenerumab, crenezumab, ponezumab, lecanemab (BAN2401), aducanumab. In some embodiments, the anti-amyloid beta antibody or antigen-binding fragment thereof comprises the heavy chain of bapineuzumab. In some embodiments, the anti-amyloid beta antibody or antigen-binding fragment thereof comprises the heavy chain of solanezumab. In some embodiments, the anti-amyloid beta antibody or antigen-binding fragment thereof comprises the heavy chain of gantenerumab. In some embodiments, the anti-amyloid beta antibody or antigen-binding fragment thereof comprises the heavy chain of crenezumab. In some embodiments, the anti-amyloid beta antibody or antigen-binding fragment thereof comprises the heavy chain of ponezumab. In some embodiments, the anti-amyloid beta antibody or antigen-binding fragment thereof comprises the heavy chain of lecanemab (BAN2401). In some embodiments, the anti-amyloid beta antibody or antigen-binding fragment thereof comprises the heavy chain of aducanumab.

In some embodiments, the anti-amyloid beta antibody or antigen-binding fragment thereof comprises the light chain of any one of bapineuzumab, solanezumab, gantenerumab, crenezumab, ponezumab, lecanemab (BAN2401), aducanumab. In some embodiments, the anti-amyloid beta antibody or antigen-binding fragment thereof comprises the light chain of bapineuzumab. In some embodiments, the anti-amyloid beta antibody or antigen-binding fragment thereof comprises the light chain of solanezumab. In some embodiments, the anti-amyloid beta antibody or antigen-binding fragment thereof comprises the light chain of gantenerumab. In some embodiments, the anti-amyloid beta antibody or antigen-binding fragment thereof comprises the light chain of crenezumab. In some embodiments, the anti-amyloid beta antibody or antigen-binding fragment thereof comprises the light chain of ponezumab. In some embodiments, the anti-amyloid beta antibody or antigen-binding fragment thereof comprises the light chain of lecanemab (BAN2401). In some embodiments, the anti-amyloid beta antibody or antigen-binding fragment thereof comprises the light chain of aducanumab.

In some embodiments, the anti-amyloid beta antibody or antigen-binding fragment thereof comprises the heavy chain and light chain of any one of bapineuzumab, solanezumab, gantenerumab, crenezumab, ponezumab, lecanemab (BAN2401), aducanumab. In some embodiments, the anti-amyloid beta antibody or antigen-binding fragment thereof comprises the heavy chain and light chain of bapineuzumab. In some embodiments, the anti-amyloid beta antibody or antigen-binding fragment thereof comprises the heavy chain and light chain of solanezumab. In some embodiments, the anti-amyloid beta antibody or antigen-binding fragment thereof comprises the heavy chain and light chain of gantenerumab. In some embodiments, the anti-amyloid beta antibody or antigen-binding fragment thereof comprises the heavy chain and light chain domain of crenezumab. In some embodiments, the anti-amyloid beta antibody or antigen-binding fragment thereof comprises the heavy chain and light chain of ponezumab. In some embodiments, the anti-amyloid beta antibody or antigen-binding fragment thereof comprises the heavy chain and light chain of lecanemab (BAN2401). In some embodiments, the anti-amyloid beta antibody or antigen-binding fragment thereof comprises the heavy chain and light chain domain of aducanumab.

In some embodiments, the anti-amyloid beta antibody comprises a heavy chain comprising the amino acid sequence of SEQ ID NO: 9. In some embodiments, the anti-amyloid beta antibody comprises a light chain comprising the amino acid sequence of SEQ ID NO: 10. In some embodiments, the anti-amyloid beta antibody comprises a heavy chain comprising the amino acid sequence of SEQ ID NO: 9, and a light chain comprising the amino acid sequence of SEQ ID NO: 10.

In some embodiments, the anti-amyloid beta antibody comprises a heavy chain comprising the amino acid sequence of SEQ ID NO: 19. In some embodiments, the anti-amyloid beta antibody comprises a light chain comprising the amino acid sequence of SEQ ID NO: 20. In some embodiments, the anti-amyloid beta antibody comprises a heavy chain comprising the amino acid sequence of SEQ ID NO: 19, and a light chain comprising the amino acid sequence of SEQ ID NO: 20.

In some embodiments, the anti-amyloid beta antibody comprises a heavy chain comprising the amino acid sequence of SEQ ID NO: 29. In some embodiments, the anti-amyloid beta antibody comprises a light chain comprising the amino acid sequence of SEQ ID NO: 30. In some embodiments, the anti-amyloid beta antibody comprises a heavy chain comprising the amino acid sequence of SEQ ID NO: 29, and a light chain comprising the amino acid sequence of SEQ ID NO: 30.

In some embodiments, the anti-amyloid beta antibody comprises a heavy chain comprising the amino acid sequence of SEQ ID NO: 39. In some embodiments, the anti-amyloid beta antibody comprises a light chain comprising the amino acid sequence of SEQ ID NO: 40. In some embodiments, the anti-amyloid beta antibody comprises a heavy chain comprising the amino acid sequence of SEQ ID NO: 39, and a light chain comprising the amino acid sequence of SEQ ID NO: 40.

In some embodiments, the anti-amyloid beta antibody comprises a heavy chain comprising the amino acid sequence of SEQ ID NO: 49. In some embodiments, the anti-amyloid beta antibody comprises a light chain comprising the amino acid sequence of SEQ ID NO: 50. In some embodiments, the anti-amyloid beta antibody comprises a heavy chain comprising the amino acid sequence of SEQ ID NO: 49, and a light chain comprising the amino acid sequence of SEQ ID NO: 50.

In some embodiments, the anti-amyloid beta antibody comprises a heavy chain comprising the amino acid sequence of SEQ ID NO: 59. In some embodiments, the anti-amyloid beta antibody comprises a light chain comprising the amino acid sequence of SEQ ID NO: 60. In some embodiments, the anti-amyloid beta antibody comprises a heavy chain comprising the amino acid sequence of SEQ ID NO: 59, and a light chain comprising the amino acid sequence of SEQ ID NO: 60.

In some embodiments, the anti-amyloid beta antibody comprises a heavy chain comprising the amino acid sequence of SEQ ID NO: 69. In some embodiments, the anti-amyloid beta antibody comprises a light chain comprising the amino acid sequence of SEQ ID NO: 70. In some embodiments, the anti-amyloid beta antibody comprises a heavy chain comprising the amino acid sequence of SEQ ID NO: 69, and a light chain comprising the amino acid sequence of SEQ ID NO: 70.

In some embodiments, the anti-amyloid beta antibody is aducanumab. In some embodiments, the anti-amyloid beta is bapineuzumab. In some embodiments, the anti-amyloid beta antibody is solanezumab. In some embodiments, the anti-amyloid beta antibody is gantenerumab. In some embodiments, the anti-amyloid beta antibody is crenezumab. In some embodiments, the anti-amyloid beta antibody is ponezumab. In some embodiments, the anti-amyloid beta antibody is lecanemab (BAN2401).

In some embodiments, the anti-amyloid beta antibody or antigen binding fragment thereof binds to amyloid beta plaques, parenchymal amyloid, cerebrovascular amyloid, or diffuse amyloid beta deposits.

The anti-amyloid beta antibody or antigen-binding fragment thereof useful in the compositions disclosed herein may comprise any of the complementarity determining regions (CDRs) (i.e., HCDRs, LCDRs), VH, VL, heavy chain or light chain sequences set forth in Tables A-G. Tables A-G provide the details of the VH, VL and of the various anti-amyloid beta antibodies.

TABLE A
Aducanumab (HCDR1-3 and LCDR1-3 based on Kabat nomenclature)

| SEQ ID NO | Description | Amino Acid Sequence |
|---|---|---|
| SEQ ID NO: 1 | HCDR1 | SYGMH |
| SEQ ID NO: 2 | HCDR2 | VIWEDGMKYYTDSVKG |
| SEQ ID NO: 3 | HCDR3 | DRGIGARRGPYYMDV |
| SEQ ID NO: 4 | LCDR1 | RASQSISSYLN |
| SEQ ID NO: 5 | LCDR2 | AASSLQS |
| SEQ ID NO: 6 | LCDR3 | QQSYSTPLI |
| SEQ ID NO: 7 | VH | QVQLVESGGGVVQPGRSLRLSCAASGFAFSSYGMHWVRQ |
| | | APGKGLEWVAVIWFDGTKKYYTDSVKGRFTISRDNSKNT |
| | | LYLQMNTLRAEDTAVYYCARDRGIGARRGPYYMDVWGKG |
| | | TTVYVSS |
| SEQ ID NO: 8 | VL | DIQMTQSPSSLSASVGDRVTITCRASQSISSYLNWYQQK |
| | | PGKAPKLLIYAASSLQSGVPSRFSGSGSGTDFTLTISSL |
| | | QPEDFATYYCQQSYSTPLTFGGGTKVEIKR |
| SEQ ID NO: 9 | Heavy chain | QVQLVESGGGVVQPGRSLRLSCAASGFAFSSYGMHWVRQ |
| | (H-Gamma-1) | APGKGLEWVAVIWFDGTKKYYTDSVKGRFTISRDNSKNT |
| | | LYLQMNTLRAEDTAVYYCARDRGIGARRGPYYMDVWGKG |
| | | TTVTVSSASTKGPSVFPLAPSSKSTSGGTAALGCLVKDY |
| | | FPEPVTVSWNSGALTSGVHTFPAVLQSSGLYSLSSVVTV |
| | | PSSSLGTQTYICNVNHKPSNTKVDKRVEPKSCDKTHTCP |
| | | PCPAPELLGGPSVFLFPPKPKDTLMISRTPEVTCVVVDV |
| | | SHEDPEVKFNWYVDGVEVHNAKTKPREEQYNSTYRVVSV |
| | | LTVLHQDWLNGKEYKCKVSNKALPAPIEKTISKAKGQPR |
| | | EPQVYTLPPSREEMTKNQVSLTCLVKGFYPSDIAVEWES |
| | | NGQPENNYKTTPPVLDSDGSFFLYSKLTVDKSRWQQGNV |
| | | FSCSVMHEALHNHYTQKSLSLSPG |
| SEQ ID NO: 10 | Light chain | DIQMTQSPSSLSASVGDRVTITCRASQSISSYLNWYQQK |
| | (L-Kappa) | PGKAPKLLIYAASSLQSGVPSRFSGSGSGTDFTLTISSL |
| | | QPEDFATYYCQQSYSTPLTFGGGTKVEIKRTVAAPSVFI |
| | | FPPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |

TABLE B
Solanezumab (HCDR1-3 and LCDR1-3 based on IMGT nomenclature)

| SEQ ID NO | Description | Amino Acid Sequence |
|---|---|---|
| SEQ ID NO: 11 | HCDR1 | GFTFSRYS |
| SEQ ID NO: 12 | HCDR2 | INSVGNST |
| SEQ ID NO: 13 | HCDR3 | ASGDY |
| SEQ ID NO: 14 | LCDR1 | QSLIYSDGNAY |
| SEQ ID NO: 15 | LCDR2 | KVS |
| SEQ ID NO: 16 | LCDR3 | SQSTHVPWT |
| SEQ ID NO: 17 | VH | EVQLVESGGGLVQPGGSLRLSCAASGFTFSRYSMSWVR |
| | | QAPGKGLELVAQINSVGNSTYYPDTVKGRFTISRDNAK |
| | | NTLYLQMNSLRAEDTAVYYCA |
| SEQ ID NO: 18 | VL | DVVMTQSPLSLPVTLGQPASISCRSSQSLIYSDGNAYL |
| | | HWFLQKPGQSPRLLIYKVSNRFSGVPDRFSGSGSGTDF |
| | | TLKISRVEAEDVGVYYCSQSTHVP |
| SEQ ID NO: 19 | Heavy | EVQLVESGGGLVQPGGSLRLSCAASGFTFSRYSMSWVR |
| | chain | QAPGKGLELVAQINSVGNSTYYPDTVKGRFTISRDNAK |
| | (H-Gamma-1) | NTLYLQMNSLRAEDTAVYYCASGDYWGQGTLVTVSSAS |
| | | TKGPSVFPLAPSSKSTSGGTAALGCLVKDYFPEPVTVS |
| | | WNSGALTSGVHTFPAVLQSSGLYSLSSVVTVPSSSLGT |
| | | QTYICNVNHKPSNTKVDKKVEPKSCDKTHTCPPCPAPE |
| | | LLGGPSVFLFPPKPKDTLMISRTPEVTCVVVDVSHEDP |
| | | EVKFNWYVDGVEVHNAKTKPREEQYNSTYRVVSVLTVL |
| | | HQDWLNGKEYKCKVSNKALPAPIEKTISKAKGQPREPQ |
| | | VYTLPPSRDELTKNQVSLTCLVKGFYPSDIAVEWESNG |
| | | QPENNYKTTPPVLDSDGSFFLYSKLTVDKSRWQQGNVF |
| | | SCSVMHEALHNHYTQKSLSLSPGK |
| SEQ ID NO: 20 | Light chain | DVVMTQSPLSLPVTLGQPASISCRSSQSLIYSDGNAYL |
| | (L-Kappa) | HWFLQKPGQSPRLLIYKVSNRFSGVPDRFSGSGSGTDF |
| | | TLKISRVEAEDVGVYYCSQSTHVPWTFGQGTKVEIKRT |
| | | VAAPSVFIFPPSDEQLKSGTASVVCLLNNFYPREAKVQ |
| | | WKVDNALQSGNSQESVTEQDSKDSTYSLSSTLTLSKAD |
| | | YEKHKVYACEVTHQGLSSPVTKSFNRGEC |

TABLE C
Crenezumab (HCDR1-3 and LCDR1-3 based on IMGT nomenclature)

| SEQ ID NO | Description | Amino Acid Sequence |
|---|---|---|
| SEQ ID NO: 21 | HCDR1 | GFTFSSYG |
| SEQ ID NO: 22 | HCDR2 | INSNGGST |
| SEQ ID NO: 23 | HCDR3 | ASGDY |
| SEQ ID NO: 24 | LCDR1 | QSLVYSNGDTY |
| SEQ ID NO: 25 | LCDR2 | KVS |
| SEQ ID NO: 26 | LCDR3 | SQSTHVPWT |
| SEQ ID NO: 27 | VH | EVQLVESGGGLVQPGGSLRLSCAASGFTFSSYGMSWVR |
| | | QAPGKGLELVASINSNGGSTYYPDSVKGRFTISRDNAK |
| | | NSLYLQMNSLRAEDTAVYYCA |
| SEQ ID NO: 28 | VL | DIVMTQSPLSLPVTPGEPASISCRSSQSLVYSNGDTYL |
| | | HWYLQKPGQSPQLLIYKVSNRFSGVPDRFSGSGSGTDF |
| | | TLKISRVEAEDVGVYYCSQSTHV |
| SEQ ID NO: 29 | Heavy | EVQLVESGGGLVQPGGSLRLSCAASGFTFSSYGMSWVR |
| | chain | QAPGKGLELVASINSNGGSTYYPDSVKGRFTISRDNAK |
| | H-Gamma-4 | NSLYLQMNSLRAEDTAVYYCASGDYWGQGTTVTVSSAS |
| | | TKGPSVFPLAPCSRSTSESTAALGCLVKDYFPEPVTVS |
| | | WNSGALTSGVHTFPAVLQSSGLYSLSSVCHVTVPSSSL |
| | | GTKTYTCNVDHKPSNTKVDKRVESKYGPPCPPCPAPEF |
| | | LGGPSVFLFPPKPKDTLMISRTPEVTCVVVDVSQEDPE |
| | | VQFNWYVDGVEVHNAKTKPREEQFNSTYRVVSVLTVLH |
| | | QDWLNGKEYKCKVSNKGLPSSIEKTISKAKGQPREPQV |
| | | YTLPPSQEEMTKNQVSLTCLVKGFYPSDIAVEWESNGQ |
| | | PENNYKTTPPVLDSDGSFFLYSRLTVDKSRWQEGNVFS |
| | | CSVMHEALHNHYTQKSLSLSLG |
| SEQ ID NO: 30 | Light chain | DIVMTQSPLSLPVTPGEPASISCRSSQSLVYSNGDTYL |
| | L-Kappa | HWYLQKPGQSPQLLIYKVSNRFSGVPDRFSGSGSGTDF |
| | | TLKISRVEAEDVGVYYCSQSTHVPWTFGQGTKVEIKRT |
| | | VAAPSVFIFPPSDEQLKSGTASVVCLLNNFYPREAKVQ |
| | | WKVDNALQSGNSQESVTEQDSKDSTYSLSSTLTLSKAD |
| | | YEKHKVYACEVTHQGLSSPVTKSFNRGEC |

TABLE D
Gantenerumab (HCDR1-3 and LCDR1-3 based on IMGT nomenclature)

| SEQ ID NO | Description | Amino Acid Sequence |
|---|---|---|
| SEQ ID NO: 31 | HCDR1 | GFTFSSYA |
| SEQ ID NO: 32 | HCDR2 | INASGTRT |
| SEQ ID NO: 33 | HCDR3 | ARGKGNTHKPYGYVRYFDV |
| SEQ ID NO: 34 | LCDR1 | QSVSSSY |
| SEQ ID NO: 35 | LCDR2 | GAS |
| SEQ ID NO: 36 | LCDR3 | LQIYNMPIT |
| SEQ ID NO: 37 | VH | QVELVESGGGLVQPGGSLRLSCAASGFTFSSYAMSWVR |
| | | QAPGKGLEWVSAINASGTRTYYADSVKGRFTISRDNSK |
| | | NTLYLQMNSLRAEDTAV |
| SEQ ID NO: 38 | VL | IVLTQSPATLSLSPGERATLSCRASQSVSSSYLAWYQQ |
| | | KPGQAPRLLIYGASSRATGVPARFSGSGSGTDFTLTIS |
| | | SLEPEDFATYYCLQIYNMP |
| SEQ ID NO: 39 | Heavy | QVELVESGGGLVQPGGSLRLSCAASGFTFSSYAMSWVR |
| | chain | QAPGKGLEWVSAINASGTRTYYADSVKGRFTISRDNSK |
| | H-Gamma-1 | NTLYLQMNSLRAEDTAVYYCARGKGNTHKPYGYVRYFD |
| | | VWGQGTLVTVSSASTKGPSVFPLAPSSKSTSGGTAALG |
| | | CLVKDYFPEPVTVSWNSGALTSGVHTFPAVLQSSGLYS |
| | | LSSVVTVPSSSLGTQTYICNVNHKPSNTKVDKKVEPKS |
| | | CDKTHTCPPCPAPELLGGPSVFLFPPKPKDTLMISRTP |
| | | EVTCVVVDVSHEDPEVKFNWYVDGVEVHNAKTKPREEQ |
| | | YNSTYRVVSVLTVLHQDWLNGKEYKCKVSNKALPAPIE |
| | | KTISKAKGQPREPQVYTLPPSRDELTKNQVSLTCLVKG |
| | | FYPSDIAVEWESNGQPENNYKTTPPVLDSDGSFFLYSK |
| | | LTVDKSRWQQGNVFSCSVMHEALHNHYTQKSLSLSPGK |
| SEQ ID NO: 40 | Light chain | DIVLTQSPATLSLSPGERATLSCRASQSVSSSYLAWYQ |
| | L-Kappa | QKPGQAPRLLIYGASSRATGVPARFSGSGSGTDFTLTI |
| | | SSLEPEDFATYYCLQIYNMPITFGQGTKVEIKRTVAAP |
| | | SVFIFPPSDEQLKSGTASVVCLLNNFYPREAKVQWKVD |
| | | NALQSGNSQESVTEQDSKDSTYSLSSTLTLSKADYEKH |
| | | KVYACEVTHQGLSSPVTKSFNRGEC |

TABLE E
Ponezumab (HCDR1-3 and LCDR1-3 based on IMGT nomenclature)

| SEQ ID NO | Description | Amino Acid Sequence |
|---|---|---|
| SEQ ID NO: 41 | HCDR1 | GYYTEAYY |
| SEQ ID NO: 42 | HCDR2 | IDPATGNT |
| SEQ ID NO: 43 | HCDR3 | ASLYSLPVY |
| SEQ ID NO: 44 | LCDR1 | QSLLYSDAKTY |
| SEQ ID NO: 45 | LCDR2 | QIS |
| SEQ ID NO: 46 | LCDR3 | LQGTHYPVL |
| SEQ ID NO: 47 | VH | QVQLVQSGAEVKKPGASVKVSCKASGYYTEAYYIHWVR |
| | | QAPGQGLEWMGRIDPATGNTKYAPRLQDRVTMTRDTST |
| | | STVYMELSSLRSEDTAVYYCA |
| SEQ ID NO: 48 | VL | DVVMTQSPLSLPVTLGQPASISCKSSQSLLYSDAKTYL |
| | | NWFQQRPGQSPRRLIYQISRLDPGVPDRFSGSGSGTDF |
| | | TLKISRVEAEDVGVYYCLQGTHYP |
| SEQ ID NO: 49 | Heavy | QVQLVQSGAEVKKPGASVKVSCKASGYYTEAYYIHWVR |
| | chain | QAPGQGLEWMGRIDPATGNTKYAPRLQDRVTMTRDTST |
| | H-Gamma-2 | STVYMELSSLRSEDTAVYYCASLYSLPVYWGQGTTVTV |
| | | SSASTKGPSVFPLAPCSRSTSESTAALGCLVKDYFPEP |
| | | VTVSWNSGALTSGVHTFPAVLQSSGLYSLSSVVTVPSS |
| | | NFGTQTYTCNVDHKPSNTKVDKTVERKCCVECPPCPAP |
| | | PVAGPSVFLFPPKPKDTLMISRTPEVTCVVVDVSHEDP |
| | | EVQFNWYVDGVEVHNAKTKPREEQFNSTFRVVSVLTVV |
| | | HQDWLNGKEYKCKVSNKGLPSSIEKTISKTKGQPREPQ |
| | | VYTLPPSREEMTKNQVSLTCLVKGFYPSDIAVEWESNG |
| | | QPENNYKTTPPMLDSDGSFFLYSKLTVDKSRWQQGNVF |
| | | SCSVMHEALHNHYTQKSLSLSPGK |
| SEQ ID NO: 50 | Light chain | DVVMTQSPLSLPVTLGQPASISCKSSQSLLYSDAKTYL |
| | L-Kappa | NWFQQRPGQSPRRLIYQISRLDPGVPDRFSGSGSGTDF |
| | | TLKISRVEAEDVGVYYCLQGTHYPVLFGQGTRLEIKRT |
| | | VAAPSVFIFPPSDEQLKSGTASVVCLLNNFYPREAKVQ |
| | | WKVDNALQSGNSQESVTEQDSKDSTYSLSSTLTLSKAD |
| | | YEKHKVYACEVTHQGLSSPVTKSFNRGEC |

TABLE F
Bapineuzumab (HCDR1-3 and LCDR1-3 based on IMGT nomenclature)

| SEQ ID NO | Description | Amino Acid Sequence |
|---|---|---|
| SEQ ID NO: 51 | HCDR1 | GFTFSNYG |
| SEQ ID NO: 52 | HCDR2 | ASIRSGGGRT |
| SEQ ID NO: 53 | HCDR3 | VRYDHYSGSSDY |
| SEQ ID NO: 54 | LCDR1 | QSLLDSDGKTY |
| SEQ ID NO: 55 | LCDR2 | LVS |
| SEQ ID NO: 56 | LCDR3 | WQGTHEPRT |
| SEQ ID NO: 57 | VH | EVQLLESGGGLVQPGGSLRLSCAASGFTFSNYGMSWVR |
| | | QAPGKGLEWVASIRSGGGRTYYSDNVKGRFTISRDNSK |
| | | NTLYLQMNSLRAEDTAVYYCVR |
| SEQ ID NO: 58 | VL | DVVMTQSPLSLPVTPGEPASISCKSSQSLLDSDGKTYL |
| | | NWLLQKPGQSPQRLIYLVSKLDSGVPDRFSGSGSGTDF |
| | | TLKISRVEAEDVGVYYCWQGTHEP |
| SEQ ID NO: 59 | Heavy | EVQLLESGGGLVQPGGSLRLSCAASGFTFSNYGMSWVR |
| | chain | QAPGKGLEWVASIRSGGGRTYYSDNVKGRFTISRDNSK |
| | H-Gamma-1 | NTLYLQMNSLRAEDTAVYYCVRYDHYSGSSDYWGQGTL |
| | | VTVSSASTKGPSVFPLAPSSKSTSGGTAALGCLVKDYF |
| | | PEPVTVSWNSGALTSGVHTFPAVLQSSGLYSLSSVVTV |
| | | PSSSLGTQTYICNVNHKPSNTKVDKKVEPKSCKTHTCP |
| | | PCPAPELLGGPSVFLFPPKPKDTLMISRTPEVTCVVVD |
| | | VSHEDPEVKFNWYVDGVEVHNAKTKPREEQYNSTYRVV |
| | | SVLTVLHQDWLNGKEYKCKVSNKALPAPIEKTISKAKG |
| | | QPREPQVYTLPPSREEMTKNQVSLTCLVKGFYPSDIAV |
| | | EWESNGQPENNYKTTPPVLDSDGSFFLYSKLTVDKSRW |
| | | QQGNVFSCSVMHEALHNKYTQKSLSLSPG |
| SEQ ID NO: 60 | Light chain | DVVMTQSPLSLPVTPGEPASISCKSSQSLLDSDGKTYL |
| | L-Kappa | NWLLQKPGQSPQRLIYLVSKLDSGVPDRFSGSGSGTDF |
| | | TLKISRVEAEDVGVYYCWQGTHFPRTFGQGTKVEIKRT |
| | | VAAPSVFIFPPSDEQLKSGTASVVCLLNNFYPREAKVQ |
| | | WKVDNALQSGNSQESVTEQDSKDSTYSL |

TABLE G
Lecanemab (HCDR1-3 and LCDR1-3 based on IMGT nomenclature)

| SEQ ID NO | Description | Amino Acid Sequence |
|---|---|---|
| SEQ ID NO: 61 | HCDR1 | GFTFSSFG |
| SEQ ID NO: 62 | HCDR2 | ISSGSSTI |
| SEQ ID NO: 63 | HCDR3 | AREGGYYYGRSYYTMDY |
| SEQ ID NO: 64 | LCDR1 | QSIVHSNGNTY |
| SEQ ID NO: 65 | LCDR2 | KVS |
| SEQ ID NO: 66 | LCDR3 | FQGSHVPPT |
| SEQ ID NO: 67 | VH | EVQLVESGGGLVQPGGSLRLSCSASGFTFSSFGMHWVR |
| | | QAPGKGLEWVAYISSGSSTIYYGDTVKGRFTISRDNAK |
| | | NSLFLQMSSLRAEDTAVYYCAR |
| SEQ ID NO: 68 | VL | DVVMTQSPLSLPVTPGAPASISCRSSQSIVHSNGNTYL |
| | | EWYLQKPGQSPKLLIYKVSNRFSGVPDRFSGSGSGTDF |
| | | TLRISRVEAEDVGIYYCFQGSHVP |
| SEQ ID NO: 69 | Heavy | EVQLVESGGGLVQPGGSLRLSCSASGFTFSSFGMHWVR |
| | chain | QAPGKGLEWVAYISSGSSTIYYGDTVKGRFTISRDNAK |
| | H-Gamma-1 | NSLFLQMSSLRAEDTAVYYCAREGGYYYGRSYYTMDYW |
| | | GQGTTVTVSSASTKGPSVFPLAPSSKSTSGGTAALGCL |
| | | VKDYFPEPVTVSWNSGALTSGVHTFPAVLQSSGLYSLS |
| | | SVVTVPSSSLGTQTYICNVNHKPSNTKVDKRVEPKSCD |
| | | KTHTCPPCPAPELLGGPSVFLFPPKPKDTLMISRTPEV |
| | | TCVVVDVSHEDPEVKFNWYVDGVEVHNAKTKPREEQYN |
| | | STYRVVSVLTVLHQDWLNGKEYKCKVSNKALPAPIEKT |
| | | ISKAKGQPREPQVYTLPPSREEMTKNQVSLTCLVKGFY |
| | | PSDIAVEWESNGQPENNYKTTPPVLDSDGSFFLYSKLT |
| | | VDKSRWQQGNVFSCSVMHEALHNHYTQKSLSLSPGK |
| SEQ ID NO: 70 | Light chain | DVVMTQSPLSLPVTPGAPASISCRSSQSIVHSNGNTYL |
| | L-Kappa | EWYLQKPGQSPKLLIYKVSNRFSGVPDRFSGSGSGTDF |
| | | TLRISRVEAEDVGIYYCFQGSHVPPTFGPGTKLEIKRT |
| | | VAAPSVFIFPPSDEQLKSGTASVVCLLNNFYPREAKVQ |
| | | WKVDNALQSGNSQESVTEQDSKDSTYSLSSTLTLSKAD |
| | | YEKHKVYACEVTHQGLSSPVTKSFNRGEC |

In some embodiments, the anti-amyloid beta antibody or antigen-binding fragment thereof comprises a human IgG1, IgG2, IgG3, or IgG4 constant domain. In some embodiments, the anti-amyloid beta antibody or antigen-binding fragment thereof comprises a human IgG1 constant region. In some embodiments, the anti-amyloid beta antibody or antigen-binding fragment thereof comprises a human IgG2 constant region. In some embodiments, the anti-amyloid beta antibody or antigen-binding fragment thereof comprises a human IgG3 constant region. In some embodiments, the anti-amyloid beta antibody or antigen-binding fragment thereof comprises a human IgG4 constant region.

In some embodiments, the anti-amyloid beta antibody or antigen-binding fragment thereof is a human anti-amyloid beta antibody or antigen-binding fragment thereof.

In some embodiments, the antigen-binding fragment is a single chain antibody, Fv, Fab, Fab′, F(ab′)₂, Fd, single chain Fv molecule (scFv), bispecific single chain Fv dimer, diabody, domain-deleted antibody or single domain antibody (dAb). In some embodiments, the anti-amyloid beta antibody is a whole antibody. In some embodiments, an anti-amyloid beta antibody is a single chain antibody. In some embodiments, an anti-amyloid beta antibody is a scFv. In some embodiments, an anti-amyloid beta antibody is a Fab. In some embodiments, an anti-amyloid beta antibody is a F(ab′)₂. In some embodiments, an anti-amyloid beta antibody is a Fv. In some embodiments, an anti-amyloid beta antibody is a Fd. In some embodiments, an anti-amyloid beta antibody is a bispecific single chain Fv dimer. In some embodiments, an anti-amyloid beta antibody is a diabody. In some embodiments, an anti-amyloid beta antibody is a single domain antibody (dAb). In some embodiments, an anti-amyloid beta antibody is a bispecific antibody.

In some embodiments, an anti-amyloid beta antibody, or an antigen-binding fragment thereof, of the disclosure is human. In some embodiments, the human anti-amyloid beta, or an antigen-binding fragment thereof, comprises a human IgH chain and a human Igκ chain. In some embodiments, the human anti-amyloid beta, or an antigen-binding fragment thereof, comprises a human IgH chain and a human Igλ chain. In some embodiments, the isotype of the anti-amyloid beta antibody is selected from IgM, IgD, IgG (such as IgG1, IgG2, IgG3, and IgG4), IgA and IgE. In some embodiments, the isotype of the anti-amyloid beta antibody is selected from IgG1, IgG2, IgG3, and IgG4.

In some embodiments, the anti-amyloid beta antibody binds an Fc receptor (FcR) selected from an FcγR, an FcεR, and an FcαR. In some embodiments, the anti-amyloid beta antibody binds an FcγR selected from FcγRI (CD64), FcγRII (CD32), and FcγRIII (CD16), including isoforms thereof. In some embodiments, the Fc region of the anti-amyloid beta antibody comprises a mutation so that it preferentially binds a particular FcγR.

In some embodiments, the anti-amyloid beta antibody or antigen binding fragment thereof binds to amyloid beta plaques, parenchymal amyloid, cerebrovascular amyloid, or diffuse amyloid beta deposits.

The following formulation examples illustrate representative pharmaceutical compositions that may be prepared in accordance with this disclosure. The present disclosure, however, is not limited to the following pharmaceutical compositions.

Exemplary Formulation 1—Tablets: A compound described herein (e.g., a compound of Formula (I), or pharmaceutically acceptable salt thereof), may be admixed as a dry powder with a dry gelatin binder in an approximate 1:2 weight ratio. A minor amount of magnesium stearate is added as a lubricant. The mixture is formed into 240-270 mg tablets (80-90 mg of active compound per tablet) in a tablet press.

Exemplary Formulation 2—Capsules: A compound described herein (e.g., a compound of Formula (I), or pharmaceutically acceptable salt thereof), may be admixed as a dry powder with a starch diluent in an approximate 1:1 weight ratio. The mixture is filled into 250 mg capsules (125 mg of active compound per capsule).

Exemplary Formulation 3—Liquid: A compound described herein (e.g., a compound of Formula (I), or pharmaceutically acceptable salt thereof), (125 mg) may be admixed with sucrose (1.75 g) and xanthan gum (4 mg) and the resultant mixture may be blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color are diluted with water and added with stirring. Sufficient water may then be added to produce a total volume of 5 mL.

Exemplary Formulation 4—Tablets: A compound described herein (e.g., a compound of Formula (I) or pharmaceutically acceptable salt thereof), may be admixed as a dry powder with a dry gelatin binder in an approximate 1:2 weight ratio. A minor amount of magnesium stearate is added as a lubricant. The mixture is formed into 450-900 mg tablets (150-300 mg of active compound) in a tablet press.

Exemplary Formulation 5—Injection: A compound described herein (e.g., a compound of Formula (I), or pharmaceutically acceptable salt thereof), may be dissolved or suspended in a buffered sterile saline injectable aqueous medium to a concentration of approximately 5 mg/mL.

Exemplary Formulation 6—Tablets: A compound described herein (e.g., a compound of Formula (I) or pharmaceutically acceptable salt thereof), may be admixed as a dry powder with a dry gelatin binder in an approximate 1:2 weight ratio. A minor amount of magnesium stearate is added as a lubricant. The mixture is formed into 90-150 mg tablets (30-50 mg of active compound per tablet) in a tablet press.

Exemplary Formulation 7—Tablets: A compound described herein (e.g., a compound of Formula (I) or pharmaceutically acceptable salt thereof), may be admixed as a dry powder with a dry gelatin binder in an approximate 1:2 weight ratio. A minor amount of magnesium stearate is added as a lubricant. The mixture is formed into 30-90 mg tablets (10-30 mg of active compound per tablet) in a tablet press.

Exemplary Formulation 8—Tablets: A compound described herein (e.g., a compound of Formula (I), or pharmaceutically acceptable salt thereof), may be admixed as a dry powder with a dry gelatin binder in an approximate 1:2 weight ratio. A minor amount of magnesium stearate is added as a lubricant. The mixture is formed into 0.3-30 mg tablets (0.1-10 mg of active compound per tablet) in a tablet press.

Exemplary Formulation 9—Tablets: A compound described herein (e.g., a compound of Formula (I) or pharmaceutically acceptable salt thereof), may be admixed as a dry powder with a dry gelatin binder in an approximate 1:2 weight ratio. A minor amount of magnesium stearate is added as a lubricant. The mixture is formed into 150-240 mg tablets (50-80 mg of active compound per tablet) in a tablet press.

Exemplary Formulation 10—Tablets: A compound described herein (e.g., a compound of Formula (I), or pharmaceutically acceptable salt thereof), may be admixed as a dry powder with a dry gelatin binder in an approximate 1:2 weight ratio. A minor amount of magnesium stearate is added as a lubricant. The mixture is formed into 270-450 mg tablets (90-150 mg of active compound per tablet) in a tablet press.

Injection dose levels range from about 0.1 mg/kg/hour to at least 10 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 2 g/day for a 40 to 80 kg human patient.

For the prevention and/or treatment of long-term conditions the regimen for treatment usually stretches over many months or years so oral dosing is preferred for patient convenience and tolerance. With oral dosing, one to five and especially two to four and typically three oral doses per day are representative regimens. Using these dosing patterns, each dose provides from about 0.01 to about 20 mg/kg of the compound provided herein, with preferred doses each providing from about 0.1 to about 10 mg/kg, and especially about 1 to about 5 mg/kg.

Transdermal doses are generally selected to provide similar or lower blood levels than are achieved using injection doses.

When used to prevent the onset of a CNS-disorder, the compounds provided herein will be administered to a subject at risk for developing the condition, typically on the advice and under the supervision of a physician, at the dosage levels described above. Subjects at risk for developing a particular condition generally include those that have a family history of the condition, or those who have been identified by genetic testing or screening to be particularly susceptible to developing the condition.

### Methods of Treatment and Use

Compounds of the present disclosure (e.g., a compound of Formula (I), and pharmaceutically acceptable salts thereof), as described herein, are generally designed to be positive allosteric modulators of NMDA function, and therefore are useful for the treatment and prevention of, e.g., CNS-related conditions in a subject.

In some embodiments, the compounds described herein (e.g., a compound of Formula (I), and pharmaceutically acceptable salts thereof), as described herein, are generally designed to penetrate the blood brain barrier (e.g., designed to be transported across the blood brain barrier). In certain embodiments, the present disclosure, e.g., a compound of Formula (I), or pharmaceutically acceptable salt thereof, may act as positive allosteric modulators (PAM) of NMDA, and potentiate NMDA receptor function.

In one aspect, the disclosure provides a method for treating a disease, disorder or condition requiring positive allosteric NMDA modulation in a subject, comprising administering to the subject an effective amount of a compound or pharmaceutically acceptable salt thereof, or a pharmaceutical composition as disclosed herein.

In one aspect, the disclosure provides a method for treating a CNS-related condition in a subject, comprising administering to the subject an effective amount of a compound or pharmaceutically acceptable salt thereof, or a pharmaceutical composition as disclosed herein.

In one aspect, the disclosure provides a method for preventing a disease, disorder or condition requiring positive allosteric NMDA modulation in a subject, comprising administering to the subject an effective amount of a compound or pharmaceutically acceptable salt thereof, or a pharmaceutical composition as disclosed herein.

In one aspect, the disclosure provides a method for preventing a CNS-related condition in a subject, comprising administering to the subject an effective amount of a compound or pharmaceutically acceptable salt thereof, or a pharmaceutical composition as disclosed herein.

In one aspect, the disclosure provides a method for inducing sedation or anesthesia in a subject, comprising administering to the subject an effective amount of a compound or pharmaceutically acceptable salt thereof, or a pharmaceutical composition as disclosed herein.

In one aspect, the disclosure provides a compound or pharmaceutically acceptable salt thereof, or pharmaceutical composition as disclosed herein for use in treating a disease, disorder or condition requiring positive allosteric NMDA modulation in a subject.

In one aspect, the disclosure provides a compound or pharmaceutically acceptable salt thereof, or pharmaceutical composition as disclosed herein for use in treating a CNS-related condition in a subject.

In one aspect, the disclosure provides a compound or pharmaceutically acceptable salt thereof, or pharmaceutical composition as disclosed herein for use in preventing a disease, disorder or condition requiring positive allosteric NMDA modulation in a subject.

In one aspect, the disclosure provides a compound or pharmaceutically acceptable salt thereof, or pharmaceutical composition as disclosed herein for use in preventing a CNS-related condition in a subject.

In one aspect, the disclosure provides a compound or pharmaceutically acceptable salt thereof, or pharmaceutical composition as disclosed herein for use in inducing sedation or anesthesia in a subject.

In one aspect, the disclosure provides the use of a compound or pharmaceutically acceptable salt thereof, or pharmaceutical composition as disclosed herein for the manufacture of a medicament for treating a disease, disorder or condition requiring positive allosteric NMDA modulation in a subject.

In one aspect, the disclosure provides the use compound or pharmaceutically acceptable salt thereof, or pharmaceutical composition as disclosed herein for the manufacture of a medicament for treating a CNS-related condition in a subject.

In one aspect, the disclosure provides the use of a compound or pharmaceutically acceptable salt thereof, or pharmaceutical composition as disclosed herein for the manufacture of a medicament for preventing a disease, disorder or condition requiring positive allosteric NMDA modulation in a subject.

In one aspect, the disclosure provides the use compound or pharmaceutically acceptable salt thereof, or pharmaceutical composition as disclosed herein for the manufacture of a medicament for preventing a CNS-related condition in a subject.

In one aspect, the disclosure provides the use of compound or pharmaceutically acceptable salt thereof, or pharmaceutical composition as disclosed herein for the manufacture of a medicament for inducing sedation or anesthesia in a subject.

In some embodiments, the method of treating a disease, disorder or condition comprises administering a compound of the disclosure in combination with a) an anti-amyloid beta antibody or antigen-binding fragment thereof. In some embodiments, the method of preventing a disease, disorder or condition comprises administering a compound of the disclosure in combination with a) an anti-amyloid beta antibody or antigen-binding fragment thereof.

In some embodiments, the disorder is cancer. In some embodiments, the disorder is diabetes. In some embodiments, the disorder is a sterol synthesis disorder. In some embodiments, the disorder is a gastrointestinal (GI) disorder, e.g., constipation, irritable bowel syndrome (IBS), inflammatory bowel disease (IBD) (e.g., ulcerative colitis, Crohn's disease), structural disorders affecting the GI, anal disorders (e.g., hemorrhoids, internal hemorrhoids, external hemorrhoids, anal fissures, perianal abscesses, anal fistula), colon polyps, cancer, colitis. In some embodiments, the disorder is inflammatory bowel disease.

In some embodiments, the disorder is Smith-Lemli-Opitz Syndrome (SLOS). In some embodiments, the disorder is desmosterolosis. In some embodiments, the disorder is sitosterolemia. In some embodiments, the disorder is cerebrotendinous xanthomatosis (CTX). In some embodiments, the disorder is Mevalonate Kinase Deficiency (MKD). In some embodiments, the disorder is SC4MOL gene mutation (SMO Deficiency). In some embodiments, the disorder is Niemann-Pick disease. In some embodiments, the disorder is autism spectrum disorder (ASD). In some embodiments, the disorder is associated with phenylketonuria.

Exemplary conditions related to positive NMDA-modulation includes, but are not limited to, gastrointestinal (GI) disorder, e.g., constipation, irritable bowel syndrome (IBS), inflammatory bowel disease (IBD) (e.g., ulcerative colitis, Crohn's disease), structural disorders affecting the GI, anal disorders (e.g., hemorrhoids, internal hemorrhoids, external hemorrhoids, anal fissures, perianal abscesses, anal fistula), colon polyps, cancer, colitis, and CNS conditions, e.g., as described herein.

Exemplary CNS conditions related to positive NMDA-modulation include, but are not limited to, adjustment disorders, anxiety disorders (including obsessive-compulsive disorder, posttraumatic stress disorder, social phobia, generalized anxiety disorder), cognitive disorders (including Alzheimer's disease and other forms of dementia (e.g., frontotemporal dementia)), dissociative disorders, eating disorders, mood disorders (including depression (e.g., postpartum depression), bipolar disorder, dysthymic disorder, suicidality), schizophrenia or other psychotic disorders (including schizoaffective disorder), sleep disorders (including insomnia), substance abuse-related disorders, personality disorders (including obsessive-compulsive personality disorder), autism spectrum disorders (including those involving mutations to the Shank group of proteins (e.g., Shank3)), neurodevelopmental disorders (including Rett syndrome), multiple sclerosis, sterol synthesis disorders, pain (including acute and chronic pain; headaches, e.g., migraine headaches), seizure disorders (including status epilepticus and monogenic forms of epilepsy such as Dravet's disease, and Tuberous Sclerosis Complex (TSC)), stroke, traumatic brain injury, movement disorders (including Huntington's disease and Parkinson's disease) and tinnitus.

In certain embodiments, the compound of the present disclosure, e.g., a compound of Formula (I), and pharmaceutically acceptable salts thereof, can be used to induce sedation or anesthesia. In certain embodiments, the compound described herein (e.g., a compound of Formula (I), and pharmaceutically acceptable salts thereof), is useful in the treatment or prevention of adjustment disorders, anxiety disorders, cognitive disorders, dissociative disorders, eating disorders, mood disorders, schizophrenia or other psychotic disorders, sleep disorders, substance-related disorders, personality disorders, autism spectrum disorders, neurodevelopmental disorders, sterol synthesis disorders, pain, seizure disorders, stroke, traumatic brain injury, movement disorders and vision impairment, hearing loss, and tinnitus. In some embodiments, the disorder is Huntington's disease. In some embodiments, the disorder is Parkinson's disease. In some embodiments, the disorder is an inflammatory disease (e.g., lupus).

In another aspect, provided is a method of treating or preventing brain excitability in a subject susceptible to or afflicted with a condition associated with brain excitability, comprising administering to the subject an effective amount of a compound of the present disclosure, e.g., a compound of Formula (I) or a pharmaceutically acceptable salt thereof.

In yet another aspect, the present disclosure provides a combination of a compound of the present disclosure, e.g., a compound of Formula (I), or pharmaceutically acceptable salt thereof, and another pharmacologically active agent. The compounds provided herein can be administered as the sole active agent or they can be administered in combination with other agents. Administration in combination can proceed by any technique apparent to those of skill in the art including, for example, separate, sequential, concurrent and alternating administration.

### Diseases and Disorders

### Movement Disorders

Also described herein are methods for treating a movement disorder. As used herein, “movement disorders” refers to a variety of diseases and disorders that are associated with hyperkinetic movement disorders and related abnormalities in muscle control. Exemplary movement disorders include, but are not limited to, Parkinson's disease and Parkinsonism (defined particularly by bradykinesia), dystonia, chorea and Huntington's disease, ataxia, tremor (e.g., essential tremor), myoclonus and startle, tics and Tourette syndrome, Restless legs syndrome, stiff person syndrome, and gait disorders.

Tremor is an involuntary, at times rhythmic, muscle contraction and relaxation that can involve oscillations or twitching of one or more body parts (e.g., hands, arms, eyes, face, head, vocal folds, trunk, legs). Tremor includes hereditary, degenerative, and idiopathic disorders such as Wilson's disease, Parkinson's disease, and essential tremor, respectively; metabolic diseases (e.g., thyroid-parathyroid-, liver disease and hypoglycemia); peripheral neuropathies (associated with Charcot-Marie-Tooth, Roussy-Levy, diabetes mellitus, complex regional pain syndrome); toxins (nicotine, mercury, lead, CO, Manganese, arsenic, toluene); drug-induced (narcoleptics, tricyclics, lithium, cocaine, alcohol, adrenaline, bronchodilators, theophylline, caffeine, steroids, valproate, amiodarone, thyroid hormones, vincristine); and psychogenic disorders. Clinical tremor can be classified into physiologic tremor, enhanced physiologic tremor, essential tremor syndromes (including classical essential tremor, primary orthostatic tremor, and task- and position-specific tremor), dystonic tremor, parkinsonian tremor, cerebellar tremor, Holmes' tremor (i.e., rubral tremor), palatal tremor, neuropathic tremor, toxic or drug-induced tremor, and psychogenic tremor. Other forms of tremor include cerebellar tremor or intention tremor, dystonic tremor, essential tremor, orthostatic tremor, parkinsonian tremor, physiological tremor, psychogenic tremor, or rubral tremor.

Cerebellar tremor or intention tremor is a slow, broad tremor of the extremities that occurs after a purposeful movement. Cerebellar tremor is caused by lesions in or damage to the cerebellum resulting from, e.g., tumor, stroke, disease (e.g., multiple sclerosis, an inherited degenerative disorder).

Dystonic tremor occurs in individuals affected by dystonia, a movement disorder in which sustained involuntary muscle contractions cause twisting and repetitive motions and/or painful and abnormal postures or positions. Dystonic tremor may affect any muscle in the body. Dystonic tremors occurs irregularly and often can be relieved by complete rest.

Essential tremor or benign essential tremor is the most common type of tremor. Essential tremor may be mild and nonprogressive in some, and may be slowly progressive, starting on one side of the body but affect both sides within 3 years. The hands are most often affected, but the head, voice, tongue, legs, and trunk may also be involved. Tremor frequency may decrease as the person ages, but severity may increase. Heightened emotion, stress, fever, physical exhaustion, or low blood sugar may trigger tremors and/or increase their severity. Symptoms generally evolve over time and can be both visible and persistent following onset.

Orthostatic tremor is characterized by fast (e.g., greater than 12 Hz) rhythmic muscle contractions that occurs in the legs and trunk immediately after standing. Cramps are felt in the thighs and legs and the patient may shake uncontrollably when asked to stand in one spot. Orthostatic tremor may occur in patients with essential tremor.

Parkinsonian tremor is caused by damage to structures within the brain that control movement. Parkinsonian tremor is often a precursor to Parkinson's disease and is typically seen as a “pill-rolling” action of the hands that may also affect the chin, lips, legs, and trunk. Onset of parkinsonian tremor typically begins after age 60. Movement starts in one limb or on one side of the body and can progress to include the other side.

Physiological tremor can occur in normal individuals and have no clinical significance. It can be seen in all voluntary muscle groups. Physiological tremor can be caused by certain drugs, alcohol withdrawal, or medical conditions including an overactive thyroid and hypoglycemia. The tremor classically has a frequency of about 10 Hz.

Psychogenic tremor or hysterical tremor can occur at rest or during postural or kinetic movement. Patient with psychogenic tremor may have a conversion disorder or another psychiatric disease.

Rubral tremor is characterized by coarse slow tremor which can be present at rest, at posture, and with intention. The tremor is associated with conditions that affect the red nucleus in the midbrain, classical unusual strokes.

Parkinson's disease affects nerve cells in the brain that produce dopamine. Symptoms include muscle rigidity, tremors, and changes in speech and gait. Parkinsonism is characterized by tremor, bradykinesia, rigidity, and postural instability. Parkinsonism shares symptoms found in Parkinson's disease, but is a symptom complex rather than a progressive neurodegenerative disease.

Dystonia is a movement disorder characterized by sustained or intermittent muscle contractions causing abnormal, often repetitive movements or postures. Dystonic movements can be patterned, twisting, and may be tremulous. Dystonia is often initiated or worsened by voluntary action and associated with overflow muscle activation.

Chorea is a neurological disorder characterized by jerky involuntary movements typically affecting the shoulders, hips, and face.

Huntington's Disease is an inherited disease that causes nerve cells in the brain to waste away. Symptoms include uncontrolled movements, clumsiness, and balance problems. Huntington's disease can hinder walk, talk, and swallowing.

Ataxia refers to the loss of full control of bodily movements, and may affect the fingers, hands, arms, legs, body, speech, and eye movements.

Myoclonus and Startle is a response to a sudden and unexpected stimulus, which can be acoustic, tactile, visual, or vestibular.

Tics are an involuntary movement usually onset suddenly, brief, repetitive, but non-rhythmical, typically imitating normal behavior and often occurring out of a background of normal activity. Tics can be classified as motor or vocal, motor tics associated with movements while vocal tics associated with sound. Tics can be characterized as simple or complex. For example, simple motor tics involve only a few muscles restricted to a specific body part.

Tourette Syndrome is an inherited neuropsychiatric disorder with onset in childhood, characterized by multiple motor tics and at least one vocal tic.

Restless Legs Syndrome is a neurologic sensorimotor disorder characterized by an overwhelming urge to move the legs when at rest.

Stiff Person Syndrome is a progressive movement disorder characterized by involuntary painful spasms and rigidity of muscles, usually involving the lower back and legs. Stiff-legged gait with exaggerated lumbar hyperlordosis typically results. Characteristic abnormality on EMG recordings with continuous motor unit activity of the paraspinal axial muscles is typically observed. Variants include “stiff-limb syndrome” producing focal stiffness typically affecting distal legs and feet.

Gait disorders refer to an abnormality in the manner or style of walking, which results from neuromuscular, arthritic, or other body changes. Gait is classified according to the system responsible for abnormal locomotion, and include hemiplegic gait, diplegic gait, neuropathic gait, myopathic gait, parkinsonian gait, choreiform gait, ataxic gait, and sensory gait.

### Mood Disorders

Also provided herein are methods for treating a mood disorder, for example clinical depression, postnatal depression or postpartum depression, perinatal depression, atypical depression, melancholic depression, psychotic major depression, cationic depression, seasonal affective disorder, dysthymia, double depression, depressive personality disorder, recurrent brief depression, minor depressive disorder, bipolar disorder or manic depressive disorder, depression caused by chronic medical conditions, treatment-resistant depression, refractory depression, suicidality, suicidal ideation, or suicidal behavior.

Clinical depression is also known as major depression, major depressive disorder (MDD), severe depression, unipolar depression, unipolar disorder, and recurrent depression, and refers to a mental disorder characterized by pervasive and persistent low mood that is accompanied by low self-esteem and loss of interest or pleasure in normally enjoyable activities. Some people with clinical depression have trouble sleeping, lose weight, and generally feel agitated and irritable. Clinical depression affects how an individual feels, thinks, and behaves and may lead to a variety of emotional and physical problems. Individuals with clinical depression may have trouble doing day-to-day activities and make an individual feel as if life is not worth living.

Postnatal depression (PND) is also referred to as postpartum depression (PPD), and refers to a type of clinical depression that affects women after childbirth. Symptoms can include sadness, fatigue, changes in sleeping and eating habits, reduced sexual desire, crying episodes, anxiety, and irritability. In some embodiments, the PND is a treatment-resistant depression (e.g., a treatment-resistant depression as described herein). In some embodiments, the PND is refractory depression (e.g., a refractory depression as described herein).

In some embodiments, a subject having PND also experienced depression, or a symptom of depression during pregnancy. This depression is referred to herein as) perinatal depression. In an embodiment, a subject experiencing perinatal depression is at increased risk of experiencing PND.

Atypical depression (AD) is characterized by mood reactivity (e.g., paradoxical anhedonia) and positivity, significant weight gain or increased appetite. Patients suffering from AD also may have excessive sleep or somnolence (hypersomnia), a sensation of limb heaviness, and significant social impairment as a consequence of hypersensitivity to perceived interpersonal rejection.

Melancholic depression is characterized by loss of pleasure (anhedonia) in most or all activities, failures to react to pleasurable stimuli, depressed mood more pronounced than that of grief or loss, excessive weight loss, or excessive guilt.

Psychotic major depression (PMD) or psychotic depression refers to a major depressive episode, in particular of melancholic nature, where the individual experiences psychotic symptoms such as delusions and hallucinations.

Catatonic depression refers to major depression involving disturbances of motor behavior and other symptoms. An individual may become mute and stuporose, and either is immobile or exhibits purposeless or bizarre movements.

Seasonal affective disorder (SAD) refers to a type of seasonal depression wherein an individual has seasonal patterns of depressive episodes coming on in the fall or winter.

Dysthymia refers to a condition related to unipolar depression, where the same physical and cognitive problems are evident. They are not as severe and tend to last longer (e.g., at least 2 years).

Double depression refers to fairly depressed mood (dysthymia) that lasts for at least 2 years and is punctuated by periods of major depression.

Depressive Personality Disorder (DPD) refers to a personality disorder with depressive features.

Recurrent Brief Depression (RBD) refers to a condition in which individuals have depressive episodes about once per month, each episode lasting 2 weeks or less and typically less than 2-3 days.

Minor depressive disorder or minor depression refers to a depression in which at least 2 symptoms are present for 2 weeks.

Bipolar disorder or manic depressive disorder causes extreme mood swings that include emotional highs (mania or hypomania) and lows (depression). During periods of mania the individual may feel or act abnormally happy, energetic, or irritable. They often make poorly thought out decisions with little regard to the consequences. The need for sleep is usually reduced. During periods of depression there may be crying, poor eye contact with others, and a negative outlook on life. The risk of suicide among those with the disorder is high at greater than 6% over 20 years, while self-harm occurs in 30-40%. Other mental health issues such as anxiety disorder and substance use disorder are commonly associated with bipolar disorder.

Depression caused by chronic medical conditions refers to depression caused by chronic medical conditions such as cancer or chronic pain, chemotherapy, chronic stress.

Treatment-resistant depression refers to a condition where the individuals have been treated for depression, but the symptoms do not improve. For example, antidepressants or psychological counseling (psychotherapy) do not ease depression symptoms for individuals with treatment-resistant depression. In some cases, individuals with treatment-resistant depression improve symptoms, but come back. Refractory depression occurs in patients suffering from depression who are resistant to standard pharmacological treatments, including tricyclic antidepressants, MAOIs, SSRIs, and double and triple uptake inhibitors and/or anxiolytic drugs, as well as non-pharmacological treatments (e.g., psychotherapy, electroconvulsive therapy, vagus nerve stimulation and/or transcranial magnetic stimulation).

Suicidality, suicidal ideation, suicidal behavior refers to the tendency of an individual to commit suicide. Suicidal ideation concerns thoughts about or an unusual preoccupation with suicide. The range of suicidal ideation varies greatly, from e.g., fleeting thoughts to extensive thoughts, detailed planning, role playing, incomplete attempts. Symptoms include talking about suicide, getting the means to commit suicide, withdrawing from social contact, being preoccupied with death, feeling trapped or hopeless about a situation, increasing use of alcohol or drugs, doing risky or self-destructive things, saying goodbye to people as if they won't be seen again.

Symptoms of depression include persistent anxious or sad feelings, feelings of helplessness, hopelessness, pessimism, worthlessness, low energy, restlessness, difficulty sleeping, sleeplessness, irritability, fatigue, motor challenges, loss of interest in pleasurable activities or hobbies, loss of concentration, loss of energy, poor self-esteem, absence of positive thoughts or plans, excessive sleeping, overeating, appetite loss, insomnia, self-harm, thoughts of suicide, and suicide attempts. The presence, severity, frequency, and duration of symptoms may vary on a case to case basis. Symptoms of depression, and relief of the same, may be ascertained by a physician or psychologist (e.g., by a mental state examination).

### Anxiety Disorders

Provided herein are methods for treating anxiety disorders.

Anxiety disorder is a blanket term covering several different forms of abnormal and pathological fear and anxiety. Current psychiatric diagnostic criteria recognize a wide variety of anxiety disorders.

Generalized anxiety disorder is a common chronic disorder characterized by long-lasting anxiety that is not focused on any one object or situation. Those suffering from generalized anxiety experience non-specific persistent fear and worry and become overly concerned with everyday matters. Generalized anxiety disorder is the most common anxiety disorder to affect older adults.

In panic disorder, a person suffers from brief attacks of intense terror and apprehension, often marked by trembling, shaking, confusion, dizziness, nausea, difficulty breathing. These panic attacks, defined by the APA as fear or discomfort that abruptly arises and peaks in less than ten minutes, can last for several hours and can be triggered by stress, fear, or even exercise; although the specific cause is not always apparent. In addition to recurrent unexpected panic attacks, a diagnosis of panic disorder also requires that said attacks have chronic consequences: either worry over the attacks' potential implications, persistent fear of future attacks, or significant changes in behavior related to the attacks. Accordingly, those suffering from panic disorder experience symptoms even outside of specific panic episodes. Often, normal changes in heartbeat are noticed by a panic sufferer, leading them to think something is wrong with their heart or they are about to have another panic attack. In some cases, a heightened awareness (hypervigilance) of body functioning occurs during panic attacks, wherein any perceived physiological change is interpreted as a possible life threatening illness (i.e. extreme hypochondriasis).

Obsessive compulsive disorder is a type of anxiety disorder primarily characterized by repetitive obsessions (distressing, persistent, and intrusive thoughts or images) and compulsions (urges to perform specific acts or rituals). The OCD thought pattern may be likened to superstitions insofar as it involves a belief in a causative relationship where, in reality, one does not exist. Often the process is entirely illogical; for example, the compulsion of walking in a certain pattern may be employed to alleviate the obsession of impending harm. And in many cases, the compulsion is entirely inexplicable, simply an urge to complete a ritual triggered by nervousness. In a minority of cases, sufferers of OCD may only experience obsessions, with no overt compulsions; a much smaller number of sufferers experience only compulsions.

The single largest category of anxiety disorders is that of phobia, which includes all cases in which fear and anxiety is triggered by a specific stimulus or situation. Sufferers typically anticipate terrifying consequences from encountering the object of their fear, which can be anything from an animal to a location to a bodily fluid.

Post-traumatic stress disorder or PTSD is an anxiety disorder which results from a traumatic experience. Post-traumatic stress can result from an extreme situation, such as combat, rape, hostage situations, or even serious accident. It can also result from long term (chronic) exposure to a severe stressor, for example soldiers who endure individual battles but cannot cope with continuous combat. Common symptoms include flashbacks, avoidant behaviors, and depression.

### Epilepsy

Epilepsy is a brain disorder characterized by repeated seizures over time. Types of epilepsy can include, but are not limited to generalized epilepsy, e.g., childhood absence epilepsy, juvenile myoclonic epilepsy, epilepsy with grand-mal seizures on awakening, West syndrome, Lennox-Gastaut syndrome, partial epilepsy, e.g., temporal lobe epilepsy, frontal lobe epilepsy, benign focal epilepsy of childhood.

### Epileptogenesis

Epileptogenesis is a gradual process by which a normal brain develops epilepsy (a chronic condition in which seizures occur). Epileptogenesis results from neuronal damage precipitated by the initial insult (e.g., status epilepticus).

### Status Epilepticus (SE)

Status epilepticus (SE) can include, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges. Convulsive status epilepticus is characterized by the presence of convulsive status epileptic seizures, and can include early status epilepticus, established status epilepticus, refractory status epilepticus, super-refractory status epilepticus. Early status epilepticus is treated with a first line therapy. Established status epilepticus is characterized by status epileptic seizures which persist despite treatment with a first line therapy, and a second line therapy is administered. Refractory status epilepticus is characterized by status epileptic seizures which persist despite treatment with a first line and a second line therapy, and a general anesthetic is generally administered. Super refractory status epilepticus is characterized by status epileptic seizures which persist despite treatment with a first line therapy, a second line therapy, and a general anesthetic for 24 hours or more.

Non-convulsive status epilepticus can include, e.g., focal non-convulsive status epilepticus, e.g., complex partial non-convulsive status epilepticus, simple partial non-convulsive status epilepticus, subtle non-convulsive status epilepticus; generalized non-convulsive status epilepticus, e.g., late onset absence non-convulsive status epilepticus, atypical absence non-convulsive status epilepticus, or typical absence non-convulsive status epilepticus.

### Seizure

A seizure is the physical findings or changes in behavior that occur after an episode of abnormal electrical activity in the brain. The term “seizure” is often used interchangeably with “convulsion.” Convulsions are when a person's body shakes rapidly and uncontrollably. During convulsions, the person's muscles contract and relax repeatedly.

Based on the type of behavior and brain activity, seizures are divided into two broad categories: generalized and partial (also called local or focal). Classifying the type of seizure helps doctors diagnose whether or not a patient has epilepsy.

Generalized seizures are produced by electrical impulses from throughout the entire brain, whereas partial seizures are produced (at least initially) by electrical impulses in a relatively small part of the brain. The part of the brain generating the seizures is sometimes called the focus.

There are six types of generalized seizures. The most common and dramatic, and therefore the most well-known, is the generalized convulsion, also called the grand-mal seizure. In this type of seizure, the patient loses consciousness and usually collapses. The loss of consciousness is followed by generalized body stiffening (called the “tonic” phase of the seizure) for 30 to 60 seconds, then by violent jerking (the “clonic” phase) for 30 to 60 seconds, after which the patient goes into a deep sleep (the “postictal” or after-seizure phase). During grand-mal seizures, injuries and accidents may occur, such as tongue biting and urinary incontinence.

Absence seizures cause a short loss of consciousness (just a few seconds) with few or no symptoms. The patient, most often a child, typically interrupts an activity and stares blankly. These seizures begin and end abruptly and may occur several times a day. Patients are usually not aware that they are having a seizure, except that they may be aware of “losing time.”

Myoclonic seizures consist of sporadic jerks, usually on both sides of the body. Patients sometimes describe the jerks as brief electrical shocks. When violent, these seizures may result in dropping or involuntarily throwing objects.

Clonic seizures are repetitive, rhythmic jerks that involve both sides of the body at the same time.

Tonic seizures are characterized by stiffening of the muscles.

Atonic seizures consist of a sudden and general loss of muscle tone, particularly in the arms and legs, which often results in a fall.

Seizures described herein can include epileptic seizures; acute repetitive seizures; cluster seizures; continuous seizures; unremitting seizures; prolonged seizures; recurrent seizures; status epilepticus seizures, e.g., refractory convulsive status epilepticus, non-convulsive status epilepticus seizures; refractory seizures; myoclonic seizures; tonic seizures; tonic-clonic seizures; simple partial seizures; complex partial seizures; secondarily generalized seizures; atypical absence seizures; absence seizures; atonic seizures; benign Rolandic seizures; febrile seizures; emotional seizures; focal seizures; gelastic seizures; generalized onset seizures; infantile spasms; Jacksonian seizures; massive bilateral myoclonus seizures; multifocal seizures; neonatal onset seizures; nocturnal seizures; occipital lobe seizures; post traumatic seizures; subtle seizures; Sylvan seizures; visual reflex seizures; or withdrawal seizures. In some embodiments, the seizure is a generalized seizure associated with Dravet Syndrome, Lennox-Gastaut Syndrome, Tuberous Sclerosis Complex, Rett Syndrome or PCDH19 Female Pediatric Epilepsy.

### Sterol Synthesis Disorders

In one embodiment, described herein are methods for treating a sterol synthesis disorder. Cholesterol has an essential rule in growth and development. It is a membrane lipid and a precursor to many molecules that play important roles in cellular growth and differentiation, protein glycosylation, and signaling pathways. Biosynthesis of cholesterol involves a number of enzymes and intermediates. Disorders resulting from a deficiency in any of the enzymes involved in cholesterol biosynthesis lead to the accumulation of intermediates and imbalance in biomolecules, resulting in disorders including congenital skeletal malformations, dysmorphic facial features, psychomotor retardation, and failure to thrive. In an embodiment, a sterol synthesis disorder or symptom of a sterol synthesis disorder can be treated by administering to a subject suffering from a sterol synthesis disorder a compound described herein, such as a positive NMDA receptor modulating compound as described herein. Additional disorders are described below.

### Smith-Lemli-Opitz Syndrome

In one embodiment, described herein are methods for treating Smith-Lemli-Opitz Syndrome (or SLOS, or 7-dehydrocholesterol reductase deficiency). SLOS is an inborn error of cholesterol synthesis. In addition to microcephaly, moderate to severe intellectual disability, sensory hypersensitivity, stereotyped behaviors, dysmorphic facial features, and syndactyly of the second/third toes, a feature of the disease is reduced cerebrosterol (24(S)-hydroxycholesterol) levels. SLOS is an autosomal recessive genetic condition resulting from deficiency in the final enzyme of the cholesterol synthesis pathway, and causes low or low-normal plasma cholesterol levels and increased 7- and 8-dehydrocholesterol (DHC; 7DHC and 8DHC) levels. Common therapies currently used include dietary cholesterol supplementation, treatment with 3-hydroxy-3-methylglutaryl coenzyme A reductase inhibitors (HMG CoA reductase inhibitors, also known as statins), and treatment with agents that enhance cholesterol production and/or accretion; and to decrease the accumulation of 7DHC and 8DHC, the potentially toxic precursors of cholesterol.

### Desmosterolosis

Desmosterolosis is a deficiency in desmosterol reductase and has a similar phenotype to SLOS. In one embodiment, described herein are methods for treating desmosterolosis with compounds described herein.

### Sitosterolemia

Sitosterolemia is a rare autosomal recessive disorder caused by mutations in two ATP-binding cassette (ABC) transporter genes (ABCG5 and ABCG8). Sitosterolemia enhances the absorption of plant sterols and cholesterol from the intestines. Patients typically present with tendon and tuberous xanthomas and premature coronary artery disease. In one embodiment, described herein are methods for treating sitosterolemia with compounds described herein.

### Cerebrotendinous Xanthomatosis (CTX)

In one embodiment, described herein are methods for treating cerebrotendinous xanthomatosis (also referred to as cerebral cholesterosis, or Van Bogaert-Scherer-Epstein syndrome) with compounds described herein. CTX can be caused by a mutation in the CYP27A1 gene, which produces the sterol 27-hydroxylase enzyme. Sterol 27-hydroxylase metabolizes cholesterol into bile acids (e.g., chenodeoxycholic acid) that are important in the absorption of fats in the intestine. Enzyme dysfunction can lead to cholesterol accumulation in tissues. CTX is characterized by childhood diarrhea, cataracts, tendon xanthomas, reduced mental capability and abnormal movements in adults.

### Mevalonate Kinase Deficiency Syndromes (MKD)

Mevalonate Kinase Deficiency (also referred to as mevalonic aciduria (a more severe form of MKD), or Hyper IgD Syndrome (HIDS, or hyperimmunoglobulinemia D) with period fever syndrome (a more benign form of MKD)) causes an accumulation of mevalonic acid in the urine as a result of insufficient activity of mevalonate kinase. MKD can result in developmental delay, hypotonia, anemia, hepatosplenomegaly, dysmorphic features, mental retardation, and overall failure to thrive. Mevalonic aciduria is characterized by delayed physical and mental development, failure to thrive, recurrent episodes of fever with vomiting and diarrhea, enlarged liver, spleen and lymph nodes, microcephaly (small head size), cataract, low muscle tone, short statute, distinct facial features, ataxia, and anemia. HIDS is characterized by recurrent episodes of fever associated with swollen lymph nodes, joint pain, gastrointestinal issues and skin rash. In one embodiment, described herein are methods for treating MKD with the compounds described herein.

### SC4MOL Gene Mutation (SMO Deficiency)

SC4MOL gene deficiency is a genetic disorder in the cholesterol biosynthesis pathway (e.g., mutations in the SC4MOL gene encoding a novel sterol oxidase). SC$MOL deficiency is characterized by the accumulation of dimethyl and monomethyl sterols that can be detected in blood, skin flakes or primary skin fibroblasts. In one embodiment, described herein are methods for treating SMO deficiency with compounds described herein.

### Niemann-Pick Disease

Niemann-Pick disease is a lysosomal storage disease resulting from a genetic mutation that affects metabolism. Niemann-Pick disease leads to abnormal accumulation of cholesterol and other fatty substances (lipids) due to an inability of the body to transport the substances. The accumulation damages the affected areas.

### Autism

In one embodiment, described herein are methods for treating autism spectrum disorder or autism. Autism spectrum disorder (ASD) and autism refer to a group of complex disorders of brain development. Autism is typically characterized by difficulties in social interaction, for example in verbal and nonverbal communication. Repetitive behaviors are also often seen in individuals having autism. Autism can be associated with intellectual disability, difficulties in motor coordination and attention and physical health issues, e.g., sleep and gastrointestinal disturbances. Individuals having autism can also excel in visual skills, music, math and art. Autism can refer to autistic disorder, childhood disintegrative disorder, pervasive developmental disorder-not otherwise specified (PDD-NOS), and Asperger syndrome. Autism also refers to monogenetic causes of autism such as synaptopathies, e.g., Rett syndrome, Fragile X syndrome, Angelman syndrome.

Disorders Associated with Phenylketonuria

In one embodiment, described herein are methods for treating disorders associated with phenylketonuria (e.g., cognitive disorders) with compounds described herein. Phenylketonuria can lead to hypocholesterolemia and lowered vitamin D status. Total and low-density cholesterols and 25-hydroxy vitamin D have been found to be decreased in subjects suffering from phenylketonuria as compared with subjects not suffering from phenylketonuria (Clin. Chim. Acta 2013, 416: 54-59). 24S-hydroxycholesterol and 27S-hydroxycholesterol and 7α-hydroxycholesterol (e.g., representing peripheral and hepatic cholesterol elimination, respectively) have been shown to be significantly decreased in subjects suffering from phenylketonuria, while 7β-hydroxycholesterol (e.g., reflecting oxidative stress) was increased significantly in subjects suffering from phenylketonuria. Changes in the levels of 24S-OHC and 7β-hydroxycholesterol correlate with phenylalanine level, and 27S-hydroxycholesterol levels may correlate with the 25-hydroxy vitamin D level in subjects suffering from phenylketonuria.

### EXAMPLES

In order that the disclosure described herein may be more fully understood, the following examples are set forth. The synthetic and biological examples described in this application are offered to illustrate the compounds, pharmaceutical compositions and methods provided herein and are not to be construed in any way as limiting their scope.

### Materials and Methods

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include (but are not limited to) recrystallization, column chromatography, HPLC, or supercritical fluid chromatography (SFC). The following schemes are presented with details as to the preparation of representative compounds that have been listed herein. The compounds provided herein may be prepared from known or commercially available starting materials and reagents by one skilled in the art of organic synthesis. Exemplary chiral columns available for use in the separation/purification of the enantiomers/diastereomers provided herein include, but are not limited to, CHIRALPAK® AD-10, CHIRALCEL® OB, CHIRALCEL® OB-H, CHIRALCEL® OD, CHIRALCEL® OD-H, CHIRALCEL® OF, CHIRALCEL® OG, CHIRALCEL® OJ and CHIRALCEL® OK.

1H-NMR reported herein (e.g., for the region between δ (ppm) of about 0.5 to about 4 ppm) will be understood to be an exemplary interpretation of the NMR spectrum (e.g., exemplary peak integrations) of a compound.

Exemplary general method for preparative HPLC: Column: Waters RBridge prep m C18, 19*250 mm. Mobile phase: acetonitrile, water (NH4HCO3) (30 L water, 24 g NH4HCO3, 30 mL NH3·H2O). Flow rate: 25 mL/min.

Exemplary general method for analytical HPLC: Mobile phase: A: water (10 mM NH4HCO3), B: acetonitrile Gradient: 5%-95% B in 1.6 or 2 min Flow rate: 1.8 or 2 mL/min; Column: XBridge C18, 4.6*50 mm, 3.5 μm at 45 C.

LC-ELSD/MS Mobile Phase: 1.5 mL/4 L TFA in water (solvent A) and 0.75 mL/4 L TFA in acetonitrile (solvent B), using the elution gradient 30%-90% (solvent B) over 0.9 minutes and holding at 90% for 0.6 minutes at a flow rate of 1.2 mL/min; Column: Xtimate C18 2.1*30 mm, 3 m; Column temperature: 50° C.; PDA Wavelength: UV 220 nm; MS ionization: ESI & ELSD.

Exemplary general method for SFC: Column: CHIRALPAK® AD CSP (250 mm*30 mm, 10 m), Gradient: 45% B, A=NH3H2O, B=MeOH, flow rate: 60 mL/min. For example, AD_3_EtOH_DEA_5_40_25ML would indicate: “Column: Chiralpak AD-3 150×4.6 mm I.D., 3 um Mobile phase: A: CG2 B: ethanol (0.05% DEA) Gradient: from 5% to 40% of B in 5 min and hold 40% for 2.5 min, then 5% of B for 2.5 min Flow rate: 2.5 mL/min Column temp: 35° C.

HRMS Instrument: Agilent G230B LCMS-TOF The mobile phase: 0.1% FA in water (solvent A) and ACN (solvent B); Elution Gradient: 5%-95% (solvent B) over 3 minutes and holding at 95% for 1 minute at a flow rate of 1 mL/minute; Column: Xbridge Shield RP 18 5 m, 2.1*50 mm; Ion Source: AJS ESI source; Ion Mode: Positive; Nebulization Gas: Nitrogen; Drying Gas (N2) Flow: 8 L/min; Nebulizer Pressure: 35 psig; Gas Temperature: 325° C.; Sheath gas Temperature: 350° C.; Sheath gas flow: 11 L/min; Capillary Voltage: 3.5 KV; Fragmentor Voltage: 175 V.

### Abbreviations

PE: petroleum ether; TLC: thin layer chromatography; petroleum ether; EtOAc: ethyl acetate; THF: tetrahydrofuran; PCC: pyridinium chlorochromate; t-BuOK: potassium tert-butoxide; 9-BBN: 9-borabicyclo[3.3.1]nonane; Pd(t-Bu₃P)₂: bis(tri-tert-butylphosphine)palladium(0); AcCi: acetyl chloride; i-PrMgCl: Isopropylmagnesium chloride; TBSCl: tert-Butyl(chloro)dimethylsilane; (i-PrO)4Ti: titanium tetraisopropoxide; Me: methyl; i-Pr: iso-propyl; t-Bu: tert-butyl; Ph: phenyl; Et: ethyl; Bz: benzoyl; BzCl: benzoyl chloride; CsF: cesium fluoride; DCC: dicyclohexylcarbodiimide; DCM: dichloromethane; DMAP: 4-dimethylaminopyridine; DMP: Dess-Martin periodinane; EtMgBr: ethylmagnesium bromide; TEA: triethylamine; AlaOH: alanine; Boc: t-butoxycarbonyl. Py: pyridine; TBAF: tetra-n-butylammonium fluoride; THF: tetrahydrofuran; TBS: t-butyldimethylsilyl; TMS: trimethylsilyl; TMSCF₃: (Trifluoromethyl)trimethylsilane; Ts: p-toluenesulfonyl; Bu: butyl; Ti(OiPr)₄: tetraisopropoxytitanium; LAH: Lithium Aluminium Hydride; LDA: lithium diisopropylamide; LiOH·H₂O: lithium hydroxide hydrates; MAD: methyl aluminum bis(2,6-di-t-butyl-4-methylphenoxide); NBS: N-bromosuccinimide; Na₂SO₄: sodium sulfate; Na₂S₂O₃: sodium thiosulfate; MeCN: acetonitrile; MeOH: methanol; Boc: t-butoxycarbonyl; MTBE: methyl tert-butyl ether; K-selectride: Potassium tri(s-butyl)borohydride; PPh₃: triphenylphosphine; PPh₃O: triphenylphosphine oxide; DMF: N,N-dimethylformamide; BHT: 2,6-di-t-butyl-p-cresol (butylated hydroxytoluene); n-BuLi: normal-Butyl lithium; TsCl: 4-methylbenzene-1-sulfonyl chloride.

### Example 1: Synthesis of (3S,8S,9S,10R,13R,14S,17R)-10,13-dimethyl-3-(methyl-d3)-17-((2R,5S)-6,6,6-trifluoro-5-hydroxy-5-methylhexan-2-yl)-2,3,4,7,8,9,10,11,12,13,14,15,16,17-tetradecahydro-1H-cyclopenta[a]phenanthren-3-ol (1)

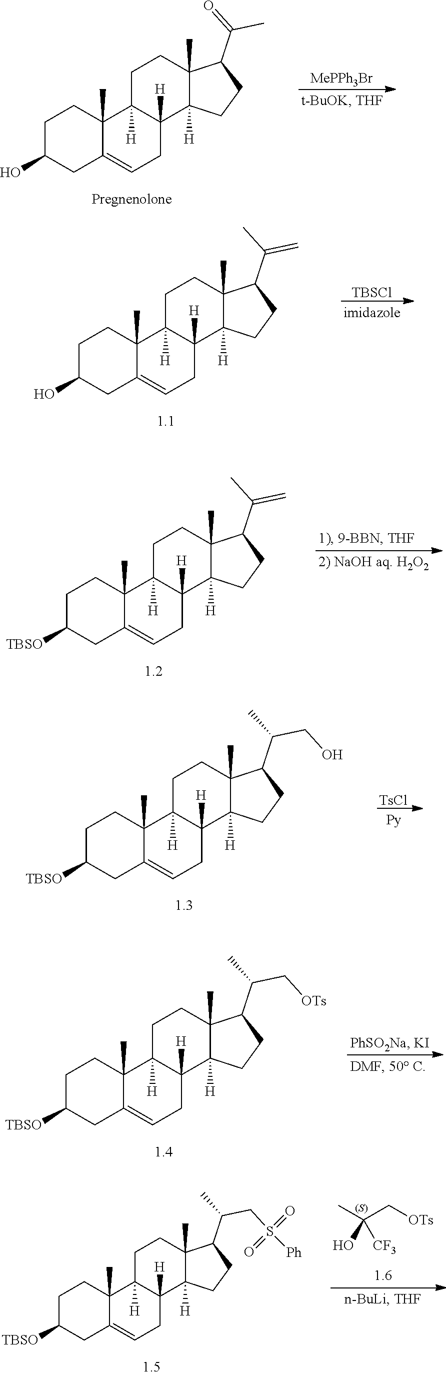

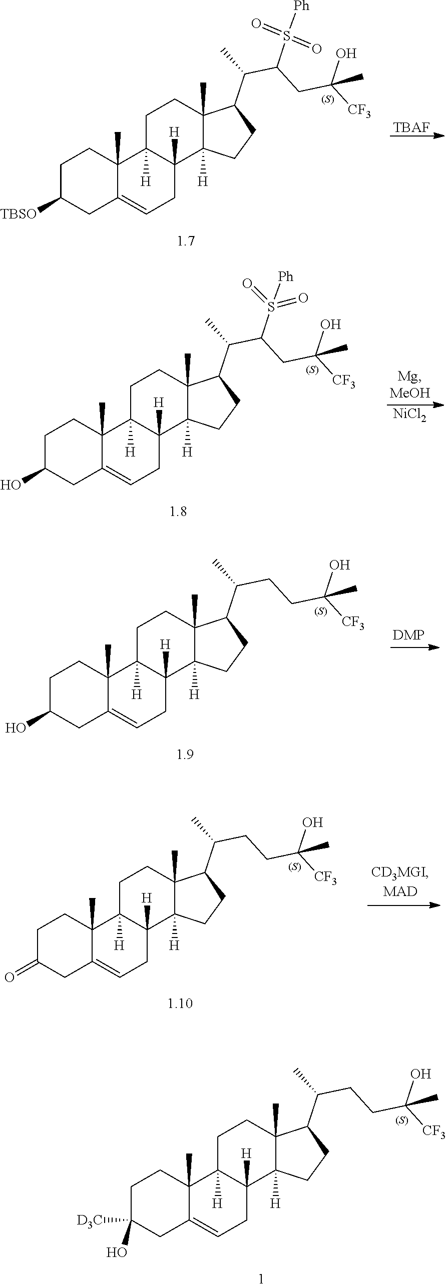

1.1, 1.2, 1.3, 1.4 and 1.5 were prepared as described in WO2017/173358 and as briefly described below.

### Synthesis of 1.1

To a suspension of PPh₃MeBr (2.13 kg, 5.97 mol) in THF (3000 mL) was added t-BuOK (688 g, 6.14 mol) at 20° C. After stirring at 50° C. for 1 h, Pregnenolone (630 g, 2.05 mol) was added at 50° C. and the reaction mixture was stirred at 50° C. for 2 h. After cooling to 20° C., the mixture was treated with NH₄Cl (10% aq., 5 L) and heptane (3.5 L) and stirred for 15 minutes. The organic layer was separated and concentrated in vacuo to give the crude material as a thick oil, which was poured into MTBE (10 L) with vigorous stirring and allowed to stir at room temperature for 16 hours. The resulting product was collected by filtration and washed with MTBE (3 L). The combined filtrate was mixed with MeOH (10 L) and concentrated to 6 L in vacuo. The resulting product was collected by filtration, washed with MeOH (3 L), and air-dried to give 700 g of wet product. The combined MeOH filtrate was concentrated in vacuo to give a thick oil. The oil was poured into MTBE (3 L) with vigorous stirring and the mixture was allowed to stir for 3 hours. The resulting product was collected by filtration and washed with MTBE (1 L). The combined filtrate was mixed with MeOH (3 L) and concentrated to 1.5 L in vacuo. The resulting product was collected by filtration, washed with MeOH (500 mL) and air-dried to give 150 g of product. The previous 700 g and 150 g batch were combined and vacuum-dried to give 1.1 (552 g, 88%). ¹H NMR (400 MHz, CDCl₃) δ 5.40-5.30 (m, 1H), 4.85 (s, 1H), 4.71 (s, 1H), 3.60-3.50 (m, 1H), 2.36-2.18 (m, 2H), 2.08-1.96 (m, 2H), 1.92-1.78 (m, 3H), 1.76 (s, 3H), 1.73-1.48 (m, 9H), 1.38-1.03 (m, 4H), 1.01 (s, 3H), 1.00-0.91 (m, 1H), 0.58 (s, 3H).

### Synthesis of 1.2

To a solution of 1.1 (4 kg, 12.7 mol) in DCM (30 L) was added imidazole (1.72 kg, 25.4 mol) and TBSCl (2.86 kg, 19.0 mol) at 25° C. After stirring at 25° C. for 16 hrs, water (10 L) was added and the organic phase was separated and concentrated to give a residue which was triturated with MeOH (15 L) at reflux to give 1.2 (5.02 kg, 92%). ¹H NMR (400 MHz, CDCl₃) δ 5.38-5.28 (m, 1H), 4.85 (s, 1H), 4.71 (s, 1H), 3.57-3.41 (m, 1H), 2.33-2.11 (m, 2H), 2.10-1.94 (m, 2H), 1.90-1.61 (m, 8H), 1.60-1.38 (m, 6H), 1.28-1.03 (m, 4H), 1.00 (s, 3H), 0.98-0.91 (m, 1H), 0.89 (s, 9H), 0.58 (s, 3H), 0.06 (s, 6H).

### Synthesis of 1.3

To a solution of 1.2 (1.69 kg, 3.94 mol) in THE (8 L) was added 9-BBN dimer (671 g, 2.75 mol) and the resulting mixture was stirred at 25° C. under N₂ for 1 h (formation of precipitate was observed). Ethanol (2.26 L, 39.4 mol) and NaOH (3.94 L, 5 M, 19.7 mol) were added and the resulting clear solution was treated dropwise with H₂O₂ (3.94 L, 10 M, 39.4 mol) at 25° C. (the inner temperature raised to reflux). After addition was complete, the mixture was cooled to 25° C. and stirred for 16 hrs, followed by addition of Na₂S₂O₃ (2.5 L, 20% aq.) and water (5 L) at 25° C. After stirring for 1 hr, the mixture was allowed to settle to a clear lower layer and an upper suspension layer. The upper suspension layer was collected and treated with water (20 L). The mixture was stirred for 15 mins and filtered. The product was washed with water to pH<9 to give the wet product, which was combined with two other batches of product from another synthesis. The wet product was dissolved in DCM (100 L) and the organic layer was separated, dried over Na₂SO₄, filtered and concentrated to 20 L. The residue was used in the next step directly. ¹H NMR (400 MHz, CDCl₃) δ 5.40-5.23 (m, 1H), 3.70-3.60 (m, 1H), 3.55-3.42 (m, 1H), 3.41-3.31 (m, 1H), 2.31-2.20 (m, 1H), 2.20-2.11 (m, 1H), 2.06-1.91 (m, 2H), 1.89-1.67 (m, 3H), 1.65-1.39 (m, 7H), 1.38-1.08 (m, 6H), 1.05 (d, J=6.4 Hz, 3H), 1.00 (s, 3H), 0.99-0.91 (m, 2H), 0.88 (s, 9H), 0.70 (s, 3H), 0.05 (s, 6H).

### Synthesis of 1.4

To a solution of 1.3 (theoretical mass: 5.2 kg, 11.6 mol) in DCM (15 L) was added N-methyl-imidazole (1.37 L, 17.4 mol) and TEA (3.2 L, 23.2 mol) at 25° C. TsCl (2.53 kg, 13.3 mol) was added portionwise to the above solution, maintaining the inner temperature between 25-30° C. The reaction mixture was stirred at 25° C. for 1 h. To the mixture was added water (10 L), citric acid (20%, 1 L) and HCl (1 M) to adjust the pH to ˜3. The organic layer was separated, washed with water (2×10 L), NaHCO₃ (saturated aq. 5 L) and brine (5 L), dried over Na₂SO₄, filtered and concentrated to give 1.4 (6.63 kg, 95% for 2 steps). ¹H NMR (400 MHz, CDCl₃) δ 7.78 (d, J=8.4 Hz, 2H), 7.34 (d, J=8.0 Hz, 2H), 5.37-5.25 (m, 1H), 3.96 (dd, J=2.8, 9.2 Hz, 1H), 3.79 (dd, J=6.4, 9.2 Hz, 1H), 3.53-3.41 (m, 1H), 2.45 (s, 3H), 2.32-2.20 (m, 1H), 2.20-2.11 (m, 1H), 2.01-1.88 (m, 2H), 1.84-1.61 (m, 4H), 1.56-1.31 (m, 6H), 1.23-1.02 (m, 5H), 1.02-0.95 (m, 7H), 0.93-0.90 (m, 1H), 0.88 (s, 9H), 0.63 (s, 3H), 0.05 (s, 6H).

### Synthesis of 1.5

To a suspension of 1.4 (2.69 kg, 4.47 mol) in DMF (25 L) was added KI (1.48 g, 8.94 mol) at 70° C. and the mixture was stirred at 70° C. for 1 h. PhSO₂Na (2.19 kg, 13.4 mol) was added and stirring was continued at 70° C. for 1 h. The mixture was poured into water (50 L) and filtered. The filter cake was washed with water (2×10 L) to give the wet product, which was combined with two other batches from previous syntheses. Half of the wet product was triturated with MeCN (20 L) at 80° C. and cooled to 30° C. The heating and cooling process was repeated two more times and the residue was collected by filtration and further triturated with MeCN/toluene (20 L, 10:1) at 80° C., filtered, washed with MeCN (3×5 L), dried in vacuo to give 1.5 (2.21 kg). Another half of the wet product was triturated with MeCN (20 L) at 80° C. and cooled to 50° C. The heating and cooling process was repeated two more times and the precipitate was collected by filtration to give 1.5 (1.92 kg). Totally 4.13 kg of the product was obtained (67% yield). ¹H NMR (400 MHz, CDCl₃) δ 8.00-7.82 (m, 2H), 7.69-7.61 (m, 1H), 7.60-7.49 (m, 2H), 5.37-5.20 (m, 1H), 3.57-3.39 (m, 1H), 3.14 (d, J=14.0 Hz, 1H), 2.85 (dd, J=9.6, 14.0 Hz, 1H), 2.35-2.05 (m, 3H), 2.02-1.88 (m, 2H), 1.85-1.62 (m, 3H), 1.61-1.32 (m, 7H), 1.29-0.91 (m, 12H), 0.88 (s, 9H), 0.65 (s, 3H), 0.05 (s, 6H).

### Synthesis of 1.6

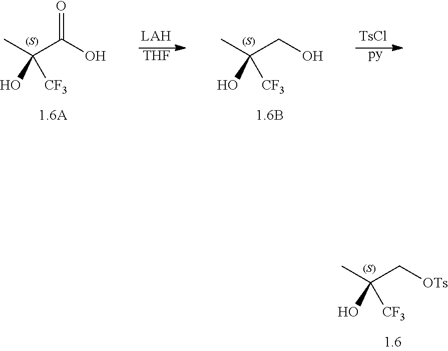

1.6B: To a suspension of LiAlH₄ (45.3 g, 1.26 mol) in THE (1 L) was added a solution of 1.6A (100 g, 632 mmol) in THF (500 mL) dropwise at 0° C. and the resulting mixture was stirred at 70° C. for 16 h. The mixture was quenched with HCl (1 L, 3 M aq.) to pH=2 and extracted with MTBE (3×500 mL). The combined organic layers were dried over anhydrous Na₂SO₄, filtered, and concentrated under reduced pressure (<40° C.) to give 1.6B (92 g). ¹H NMR (400 MHz, CDCl₃) δ 3.96-3.92 (m, 1H), 3.58-3.53 (m, 1H), 3.08 (s, 1H), 1.98-1.89 (m, 1H), 1.38 (s, 3H).

1.6: To a solution of 1.6B (50 g, 346 mmol) in pyridine (300 mL) was added 4-methylbenzene-1-sulfonyl chloride (98.9 g, 519 mmol) in portions over 5 min at 0° C. and the resulting solution was stirred at 20° C. for 16 h. The reaction mixture was quenched with 2N HCl (400 mL) to pH=1-2 at 0° C. The internal temperature was maintained below 30° C. and the mixture was extracted with MTBE (3×200 mL). The combined organic layers were dried over anhydrous Na₂SO₄, filtered, concentrated, and purified by silica gel chromatography (0˜10% EtOAc in PE) to give 1.6 (93 g, 90%, 99.42% ee). ¹H NMR (400 MHz, CDCl₃) δ 7.79 (d, J=7.6 Hz, 2H), 7.37 (d, J=8.0 Hz, 2H), 4.13-4.03 (m, 2H), 2.99 (s, 1H), 2.46 (s, 3H), 1.37 (s, 3H).

### Synthesis of 1.7

To a solution of n-BuLi (6.27 mL, 15.7 mmol, 2.5 M) in THE (10 mL) under N₂ at −70° C. was added a suspension of 1.5 (3.60 g, 6.30 mmol) in THE (30 mL). After stirring at −70° C. for 30 mins, a solution of 1.6 (2.25 g, 7.56 mmol) in THE (10 mL) was added. The reaction was stirred at −70° C. for 10 mins and stirred at 25° C. for 16 hrs. The reaction was quenched with Sat. NH₄Cl (400 mL) and extracted with EtOAc (3×600 mL). The combined organic layers were dried over Na₂SO₄, filtered, and concentrated give 4.80 g of 1.7 which was used for next step directly. ¹H NMR (400 MHz, CDCl₃) δ_{H} 7.91 (d, J=7.6 Hz, 2H), 7.75-7.68 (m, 1H), 7.66-7.57 (m, 2H), 5.27 (m, 1H), 4.95 (s, 1H), 3.51-3.39 (m, 1H), 3.29 (d, J=7.6 Hz, 1H), 2.58 (dd, J=8.4, 16.4 Hz, 1H), 2.29-2.18 (m, 1H), 2.18-2.09 (m, 1H), 1.97-1.82 (m, 6H), 1.80-1.24 (m, 14H), 1.16-0.80 (m, 17H), 0.45-0.37 (m, 4H), 0.04 (s, 6H).

### Synthesis of 1.8

To a solution of 1.7 (4.80 g, 6.88 mmol) in THE (10 mL) was added TBAF (27.5 mmol, 27.5 mL, 1.0 M in THF) at 15° C. The reaction mixture was stirred at 15° C. for 16 hrs. The reaction mixture was stirred at 65° C. for 1 h. The reaction mixture was concentrated under vacuum to give a residue, which was dissolved in MeOH (20 mL). To the MeOH solution was added water (200 mL) dropwise. The product was filtered and washed with water (5×50 mL), dried under vacuum to give 1.8 (3.93 g).

### Synthesis of 1.9

To a solution of 1.8 (3.70 g, 6.34 mmol) and NiCl₂ (204 mg, 1.58 mmol) in dry MeOH (100 mL) and THE (50 mL) was added Mg turnings (3.84 g, 158 mol) (activated with 0.5% aqueous HCl, water, dry ethanol, and MTBE) under N₂ with stirring at 50° C. to initiate continuous hydrogen generation. The reaction mixture was stirred at 60° C. for 1 h. The reaction mixture was quenched with 2 M HCl (100 mL) at 10° C. until the solid was dissolved. The mixture was extracted with EtOAc (2×150 mL). The combined organic layer was washed with sat. NaHCO₃ (300 mL), brine (300 mL), dried over Na₂SO₄, filtered, and concentrated under vacuum to give 1.9 (2.85 g), which was purified by silica gel chromatography (PE:EtOAc=10:1) to give 1.9 (2.05 g, 68%). ¹H NMR (400 MHz, CDCl₃) δ_{H} 5.40-5.30 (m, 1H), 3.60-3.46 (m, 1H), 2.34-2.16 (m, 2H), 2.06-1.91 (m, 2H), 1.90-1.77 (m, 4H), 1.75-1.38 (m, 11H), 1.35-1.22 (m, 5H), 1.21-0.76 (m, 12H), 0.68 (s, 3H).

### Synthesis of 1.10

To a solution of 1.9 (500 mg, 1.12 mmol) in DCM (10 mL) was added DMP (2.37 g, 5.60 mmol) at 25° C. in portions. Water (40 mg) was added and the mixture was stirred at 25° C. for 30 mins. The mixture was washed with NaHCO₃ (2×20 mL, sat. aq.) and Na₂S₂O₃ (20 mL, sat. aq.), dried over Na₂SO₄, filtered, and concentrated in vacuum below 30° C. to give 1.10 (0.49 g, 99%). ¹H NMR (400 MHz, CDCl₃) δ_{H} 5.38-5.30 (m, 1H), 3.28 (dd, J=2.8 Hz, J=16.4 Hz, 1H), 2.82 (dd, J=2.0 Hz, J=16.4 Hz, 1H), 2.58-2.42 (m, 1H), 2.36-2.25 (m, 1H), 2.09-1.96 (m, 3H), 1.90-0.98 (m, 25H), 0.95 (d, J=6.8 Hz, 3H), 0.71 (s, 3H).

### Synthesis of 1

To a solution of BHT (2.44 g, 11.1 mmol) in toluene (10 mL) was added AlMe₃ (2.77 mL, 2 M in toluene, 5.55 mmol) dropwise at 10° C. The mixture was stirred at 15° C. for 1 h as a freshly prepared MAD solution. To the fresh prepared MAD solution was added a solution of 1.10 (0.490 g, 1.11 mmol) in toluene (10 mL) dropwise at −70° C. under N₂. The mixture was stirred at −70° C. for 1 h. A solution of CD₃MgI (11.1 mmol in 15 mL Et₂O) was added dropwise to the above mixture at −70° C. The mixture was stirred at −70° C. for another 1 h. The mixture was poured into citric acid (10 mL, 10% aq.) in portions. Gas was evolved. The mixture was extracted with EtOAc (2×20 mL) and the combined organic layer was concentrated in vacuum. The residue was triturated from petroleum ether (50 mL), purified by silica gel column (petroleum ether:EtOAc=15:1 to 10:1) and SFC (Column: AD (250 mm*30 mm, 5 um); Condition: Base-MeOH; Begin B: 30%; End B: 30%; Gradient Time (min): 10; Flow Rate (mL/min): 60 mL/min; Injections: 120) to give 1 (32.8 mg, 6%, deuterated ratio: 99.5%). ¹H NMR (400 MHz, CDCl₃) δ 5.35-5.26 (m, 1H), 2.48-2.35 (m, 1H), 2.05-0.92 (m, 35H), 0.68 (s, 3H). LCMS Rt=1.250 min in 2.0 min chromatography, 30-90 AB, purity 100%, MS ESI calcd. for C₂₇H₄₀D₃F₃O₂ [M+H−H₂O]⁺ 442, found 442. HRMS MS calcd. for C₂₇H₄₀D₃F₃O₂ [M+H−H₂O]⁺ 442.3371, found 442.3380.

### Example 2: Synthesis of (3S,8S,9S,10R,13R,14S,17R)-3,10,13-trimethyl-17-((2R,5S)-6,6,6-trifluoro-5-hydroxy-5-(methyl-d3)hexan-2-yl)-2,3,4,7,8,9,10,11,12,13,14,15,16,17-tetradecahydro-1H-cyclopenta[a]phenanthren-3-ol (2)

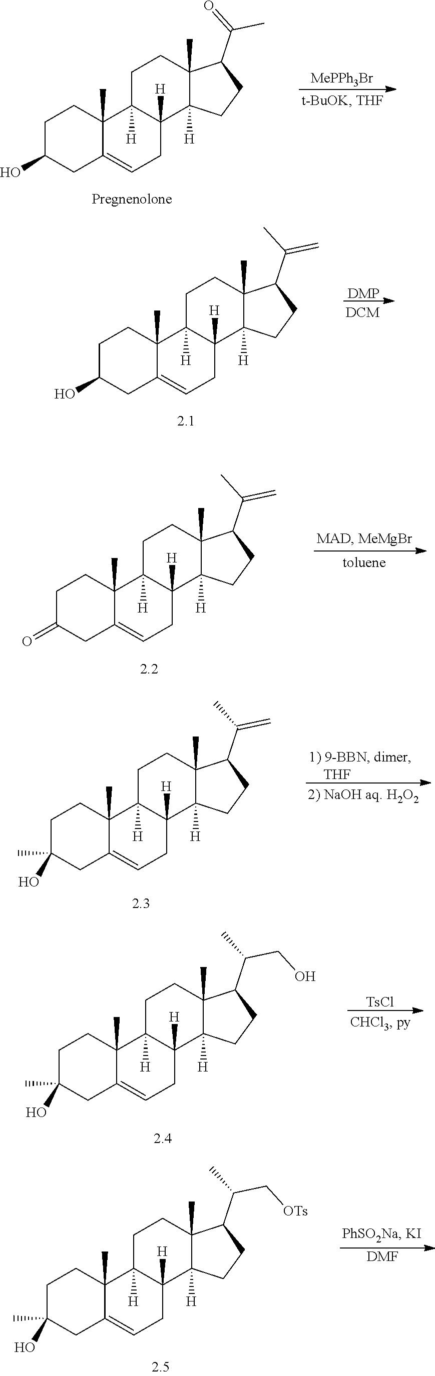

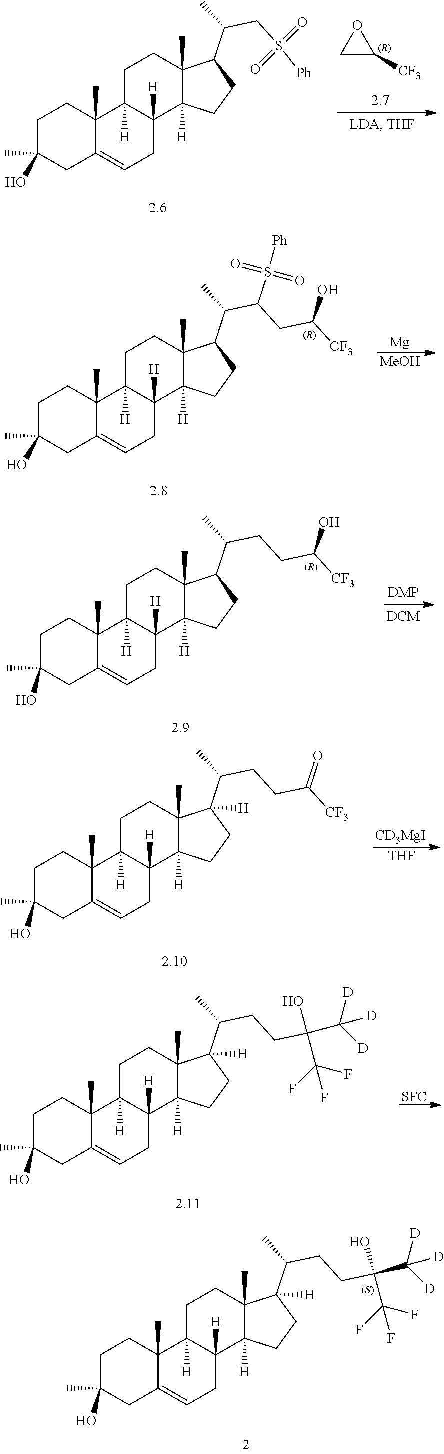

### Synthesis of 2.1

To a mixture of MePPh₃Br (1.28 kg, 3.6 mol) in THE (4.5 L) was added t-BuOK (404 g, 3.6 mol) at 15° C. under N₂ and the resulting mixture was stirred at 50° C. for 30 min. Pregnenolone (950 g, 2.9 mol) was added in portions to keep the internal temperature below 65° C. and the reaction mixture was stirred at 50° C. for 1 h. The mixture was quenched with saturated aqueous NH₄Cl (1 L) at 15° C. and the THF layer was separated. The aqueous layer was extracted with EtOAc (2×2 L), and the combined organic layers were concentrated under vacuum. The residue was purified by trituration with MeOH/H₂O (1:1, 15 L) at reflux to give 2.1 (940 g, 99%). ¹H NMR (400 MHz, CDCl₃) δ 5.40-5.32 (m, 1H), 4.85 (s, 1H), 4.71 (s, 1H), 3.58-3.46 (m, 1H), 2.36-2.16 (m, 2H), 2.08-1.94 (m, 2H), 1.92-1.62 (m, 9H), 1.61-1.39 (m, 6H), 1.29-1.03 (m, 4H), 1.01 (s, 3H), 0.99-0.91 (m, 1H), 0.59 (s, 3H).

### Synthesis of 2.2

To a solution of 2.1 (800 g, 2.54 mol) in DCM (8 L) was added DMP (2.14 kg, 5.08 mol) in portions at 35° C. and the resulting mixture was stirred for 20 min. The reaction mixture was filtered, and the filtered cake was washed with DCM (3 xl L). The combined organic layers were washed with saturated aqueous Na₂S₂O₃/NaHCO₃ (3:1, 2×1.5 L), brine (1.5 L), dried over anhydrous Na₂SO₄, filtered, and concentrated under vacuum to give 2.2 (794 g), which was used directly in the next step.

### Synthesis of 2.3

To a solution of BHT (1.97 kg, 8.94 mol) in toluene (1 L) was added AlMe₃ (2.14 L, 2.0 M in toluene, 4.28 mol) dropwise to keep the internal temperature below 25° C. under N₂ atmosphere. The resulting mixture was stirred at 25° C. for 1 h. 2.2 (794 g, 2.16 mol) in DCM (3 L) was added at −70° C. and the mixture was stirred for 1 h. MeMgBr (862 mL, 3.0 M in diethyl ether, 2.59 mol) was added at −70° C. and the reaction mixture was stirred for 10 min. The mixture was quenched with saturated aqueous critic acid (3 L) and extracted with EtOAc (2×2 L). The combined organic layers were washed with brine (2 L), dried over anhydrous Na₂SO₄, filtered, and concentrated under vacuum to give a residue, which was triturated from MeCN (3 L) at 25° C. to give 2.3 (340 g, 43%). ¹H NMR (400 MHz, CDCl₃) δ 5.34-5.26 (m, 1H), 4.85 (s, 1H), 4.71 (s, 1H), 2.50-2.35 (m, 1H), 2.07-1.94 (m, 3H), 1.91-1.84 (m, 1H), 1.83-1.63 (m, 8H), 1.58-1.33 (m, 6H), 1.27-1.05 (m, 7H), 1.02 (s, 3H), 1.00-0.92 (m, 1H), 0.58 (s, 3H).

### Synthesis of 2.4

To a mixture of 2.3 (149 g, 453 mmol) and 9-BBN dimer (127 g, 520 mmol) was added THE (1 L) at 15° C. under N₂ and the resulting mixture was stirred at 60° C. for 1 h. The mixture was cooled to 15° C. and EtOH (208 g, 4.53 mol) was added. Aqueous NaOH (906 mL, 5 M, 4.53 mol) was added dropwise at 15° C., followed by H₂O₂ (514 g, 30%, 4.53 mol) addition dropwise at 15° C. The mixture was stirred at 60° C. for 1 h. The solid was washed with ethanol (200 mL) to give a residue, which was triturated with EtOH (2.3 L) at reflux and water (2.5 L) at 80° C. successively to give 2.4 (131 g, 84%). ¹H NMR (400 MHz, CDCl₃) δ 5.35-5.24 (m, 1H), 3.67-3.61 (m, 1H), 3.42-3.33 (m, 1H), 2.50-2.35 (m, 1H), 2.07-1.92 (m, 3H), 1.88-1.65 (m, 3H), 1.60-1.38 (m, 8H), 1.37-1.26 (m, 1H), 1.26-1.08 (m, 8H), 1.05 (d, J=6.8 Hz, 3H), 1.01 (s, 3H), 1.00-0.91 (m, 2H), 0.70 (s, 3H).

### Synthesis of 2.5

To a solution of 2.4 (131 g, 378 mmol) in CHCl₃ (600 mL) and pyridine (420 mL) was added TsCl (187 g, 982 mmol) at 15° C. and the resulting mixture was stirred for 2 h. The reaction mixture was concentrated under vacuum to remove most of the CHCl₃. Water (3 L) was added, and the precipitate was filtered. The precipitate was washed with water (6×4 L), dissolved in DCM (3.5 L), dried over anhydrous Na₂SO₄, filtered, and concentrated under vacuum to give 2.5 (177 g, 94%). ¹H NMR (400 MHz, CDCl₃) δ 7.78 (d, J=8.4 Hz, 2H), 7.34 (d, J=8.0 Hz, 2H), 5.34-5.25 (m, 1H), 3.96 (dd, J=3.2, 9.6 Hz, 1H), 3.79 (dd, J=6.4, 9.2 Hz, 1H), 2.45 (s, 3H), 2.50-2.35 (m, 1H), 2.02-1.88 (m, 3H), 1.81-1.61 (m, 4H), 1.58-1.33 (m, 7H), 1.24-1.11 (m, 8H), 1.09-0.86 (m, 8H), 0.64 (s, 3H).

### Synthesis of 2.6

To a solution of 2.5 (177 g, 353 mmol) in DMF (1.8 L) was added KI (281 g, 1694 mmol) at 15° C. and the resulting mixture was stirred at 60° C. for 1 h. PhSO₂Na (211 g, 1.06 mol) was added, and the mixture was stirred at 60° C. for 2 h. The reaction mixture was cooled to 25° C. and the mixture was poured into water (20 L) and filtered. The filtered cake was washed with water (3×2 L) and dissolved in DCM (5 L). The filtrate was washed with water (2×1 L), brine (2×1 L), dried over anhydrous Na₂SO₄, filtered, and concentrated in vacuo to give a residue, which was re-crystallized from toluene (2.5 L) to give 2.6 (121 g, 73%). The re-crystallization filtrate was concentrated under vacuum to give 2.6 (20 g). ¹H NMR (400 MHz, CDCl₃) δ 7.91 (d, J=7.6 Hz, 2H), 7.69-7.61 (m, 1H), 7.61-7.53 (m, 2H), 5.31-5.24 (m, 1H), 3.14 (d, J=14 Hz, 1H), 2.85 (dd, J=9.2, 14 Hz, 1H), 2.50-2.35 (m, 1H), 2.16-2.03 (m, 1H), 2.01-1.88 (m, 3H), 1.80-1.64 (m, 3H), 1.56-1.34 (m, 6H), 1.20 (d, J=6.8 Hz, 3H), 1.17-1.11 (m, 3H), 1.10 (s, 3H), 1.08-1.01 (m, 2H), 1.00 (s, 4H), 0.98-0.87 (m, 2H), 0.65 (s, 3H).

### Synthesis of 2.7

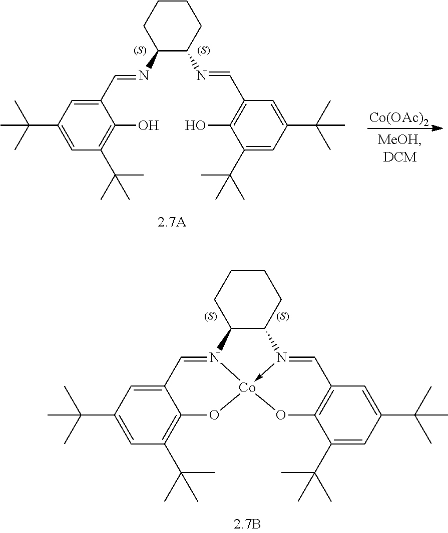

2.7B: To a solution of 2.7A (2 g, 3.65 mmol) in anhydrous DCM (30 mL) was added a solution of cobalt(II) acetate (775 mg, 4.38 mmol) in MeOH (30 mL) under nitrogen at 20° C. and the resulting mixture was stirred for 30 min at 20° C. and at 0° C. for 1 h. The precipitate was filtered, washed with cold MeOH (2×30 mL) and dried in vacuum to give 2.7B (1.6 g, 73%).

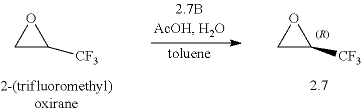

2.7: To a solution of 2.7B (1.07 g, 1.78 mmol) in toluene (30 mL) was added AcOH (1.12 g, 18.7 mmol) and the resulting mixture was stirred at 20° C. for 30 min. The solution was concentrated in vacuo. The resulting catalyst residue was dissolved in 2-(trifluoromethyl)oxirane (100 g, 892 mmol) at 20° C., the reaction mixture was cooled to 0° C., and water (8.82 g, 490 mmol) was added dropwise. The mixture was warmed to 20° C. and stirred for 48 h. 2.7 (44 g) was isolated by distillation from the reaction mixture. ¹H NMR (400 MHz, DMSO-d6) δ 3.96 (s, 1H), 3.11-2.98 (m, 2H).

### Synthesis of 2.8

To a solution of diisopropylamine (26.8 g, 265 mmol) in THE (800 mL) was added BuLi (93.2 mL, 2.5 M in hexane, 233 mmol) dropwise under N₂ at −70° C. The mixture was warmed to 0° C. and a suspension of 2.6 (50 g, 106 mmol) in THE (300 mL) was added. The mixture was stirred at −70° C. for 30 min, and a solution of 2.7 (17.8 g, 159 mmol) in THE (100 mL) was added. The mixture was stirred at −70° C. for 30 min and then warmed to 15° C. and stirred for 24 h. The reaction mixture was quenched by adding 600 mL of saturated aqueous NH₄Cl at 15° C. The organic layer was separated, and the aqueous phase was extracted with EtOAc (2×100 mL). The combined organic layers were concentrated in vacuo. The residue was dissolved in EtOAc (600 mL), washed with brine (200 mL), dried over anhydrous Na₂SO₄, filtered, concentrated, and purified by silica gel chromatography (0˜40% of EtOAc in PE) to give of 2.8 (53 g, 86%), which was used directly in the next step.

### Synthesis of 2.9

To a solution of 2.8 (53 g, 90.9 mmol) in MeOH (800 mL) was added Mg powder (25 g, 1.02 mol) in portions at 65° C. over 30 min and the resulting mixture was stirred at 65° C. for 1 h. Citric acid (20% aq., 1000 mL) was added followed by HCl (2 N, 200 mL). The mixture was extracted with PE/EtOAc (1:1, 1000 mL+200 mL) and the combined organic layers were washed with brine (200 mL), dried over anhydrous Na₂SO₄, filtered, concentrated in vacuo, purified by silica gel chromatography (PE:EtOAc=12:1 to 8:1), and re-crystallized from MeOH (250 mL) to give 2.9 (10.94 g, 27%). ¹H NMR (400 MHz, CDCl₃) δ 5.35-5.25 (m, 1H), 3.95-3.80 (m, 1H), 2.48-2.37 (m, 1H), 2.08-1.91 (m, 4H), 1.90-1.42 (m, 13H), 1.39-0.90 (m, 18H), 0.69 (s, 3H). ¹⁹F NMR (376.5 MHz, CDCl₃) δ 80.03 (s). LCMS Rt=1.240 min in 2.0 min chromatography, 30-90AB, purity 100% (ELSD), MS ESI calcd. for C₂₆H₄₀F₃O [M+H−H₂O]⁺ 425, found 425. SFC Rt=4.543 min in 10.0 min chromatography, AD_3_MeOH_DEA_5_40_25ML, purity 100% (UV 210 nm).

### Synthesis of 2.10

To a solution of 2.9 (15 g, 33.8 mmol) in DCM (200 mL) was added DMP (28.6 g, 67.6 mmol) at 25° C. and the resulting mixture was stirred for 1 h. The reaction mixture was quenched with saturated aqueous NaHCO₃ (200 mL) until the pH of the aqueous layer was 7. Saturated aqueous Na₂S₂O₃ (200 mL) was added, and the mixture was stirred at 25° C. for 1 h. The mixture was extracted with DCM (2×300 mL), dried over anhydrous Na₂SO₄, filtered, and concentrated. The product was purified by silica gel chromatography (EtOAc in PE, 10%˜15%) to give 2.10 (9 g, 61%), which was purified three additional times by silica gel chromatography (EtOAc in PE, 15%˜20%) to give 2.10 (7.1 g). 2.10 (7 g) was triturated with n-hexane (80 mL) three times at 25° C. to give 2.10 (4013.1 mg). ¹H NMR (400 MHz, CDCl₃) δ 5.34-5.26 (m, 1H), 3.18-2.54 (m, 2H), 2.47-2.36 (m, 1H), 2.03-1.92 (m, 3H), 1.90-1.58 (m, 4H), 1.57-1.24 (m, 10H), 1.22-0.90 (m, 15H), 0.68 (s, 3H). MS for C₂₆H₄₀F₃O₂ [M−H₂O+H]⁺ 423, found 423.

### Synthesis of 2.11

A solution of CD₃MgI (5.99 mmol, in 6 mL ether) was reacted with a solution of 2.10 (530 mg, 1.20 mmol) in THF (5 mL) at 0° C. and the resulting mixture was stirred at 20° C. for 16 h. The mixture was poured into saturated aqueous NH₄Cl (20 mL) at 0° C. and was extracted with EtOAc (2×20 mL). The combined organic layers were dried over anhydrous Na₂SO₄, filtered, and concentrated under vacuum. The residue was purified by silica gel chromatography (0-30% EtOAc in PE) to give 2.11 (184 mg, 33.3%).

2.11 (184 mg) was separated by SFC (Column: DAICEL CHIRALPAK AD (250 mm*30 mm, 10 um); Condition: 0.1% NH₃·H₂O MeOH; Begin B: 25%; End B: 25%; Gradient Time (min): 60; Flow Rate (ml/min): 80; Injections: 80) to give 2 (55 mg, 30.0%). 2 (46 mg, 0.100 mmol) was lyophilized to give 2 (31 mg, 67.5%). ¹H NMR (400 MHz, CDCl₃) δ_{H} 5.34-5.28 (m, 1H), 2.46-2.38 (m, 1H), 2.03-1.58 (m, 11H), 1.53-1.21 (m, 9H), 1.20-1.13 (m, 3H), 1.11 (s, 3H), 1.10-1.02 (m, 2H), 1.01 (s, 3H), 1.00-0.96 (m, 1H), 0.94 (d, J=6.4 Hz, 3H), 0.68 (s, 3H); ¹⁹F NMR (376 MHz, CDCl₃) δ_{F} −83.16 (s); LC-ELSD/MS 30-90AB_2 min_E, purity >99%, MS ESI calcd. for C₂₇H₃₉D₃F₃O [M−H₂O+H]⁺ 442.3, found 442.3.

### Example 3: An Alternative Method for Preparing 2 is Provided Below

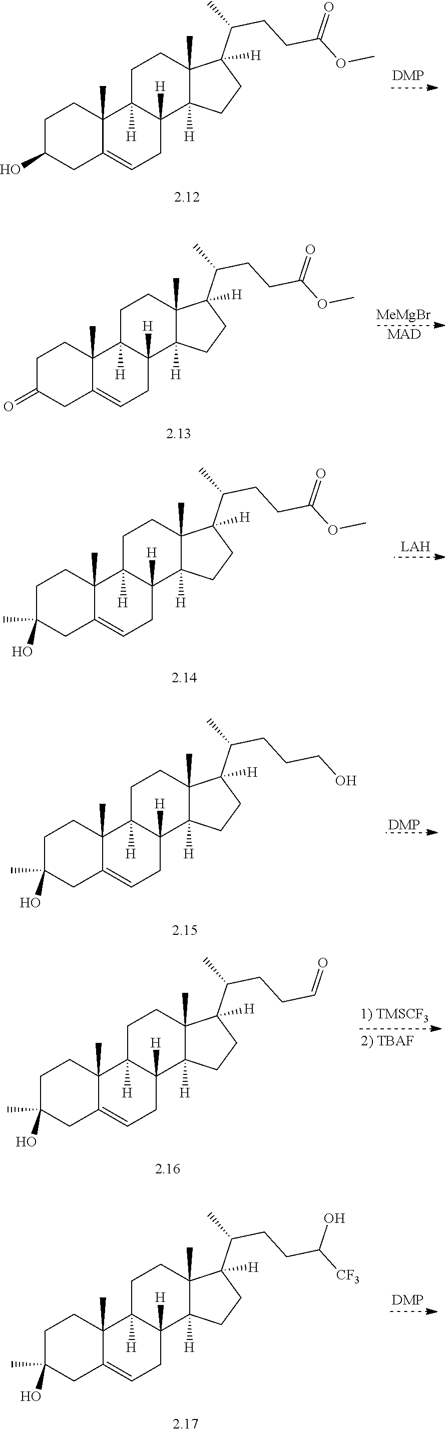

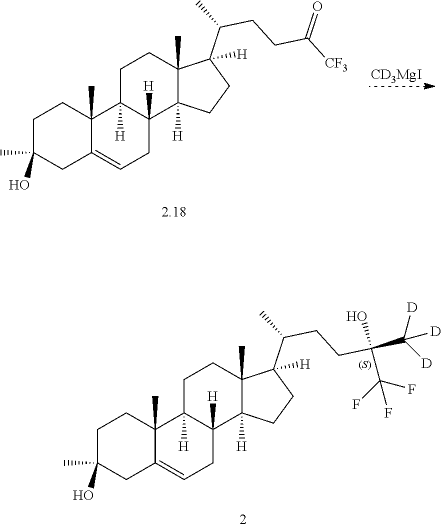

### Example 4: Synthesis of (3S,8S,9S,10R,13R,14S,17R)-3,10,13-trimethyl-17-((2R,5S)-6,6,6-trifluoro-5-hydroxy-5-methylhexan-2-yl-1,1,1-d3)-2,3,4,7,8,9,10,11,12,13,14,15,16,17-tetradecahydro-1H-cyclopenta[a]phenanthren-3-ol (3)

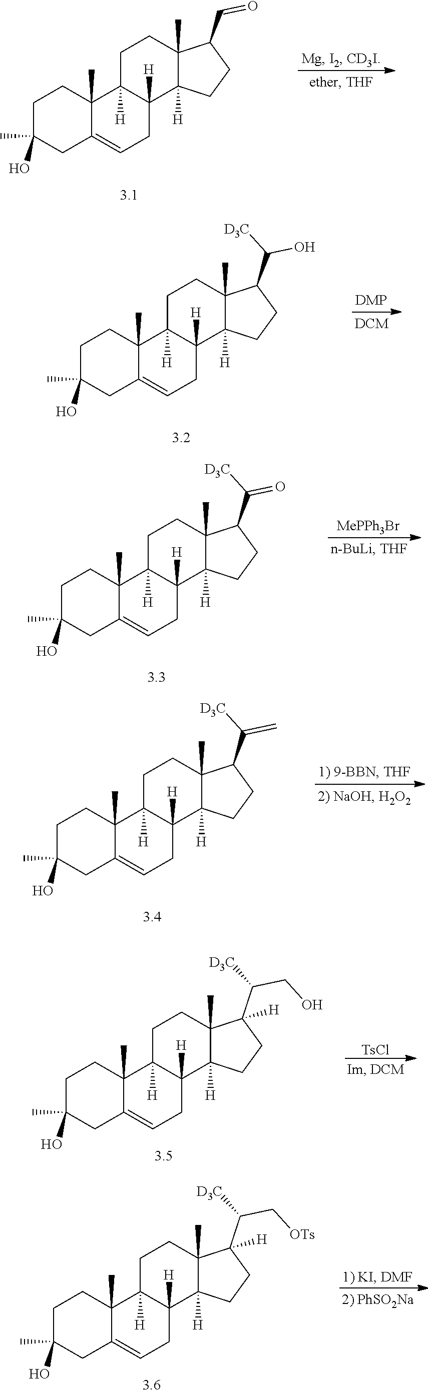

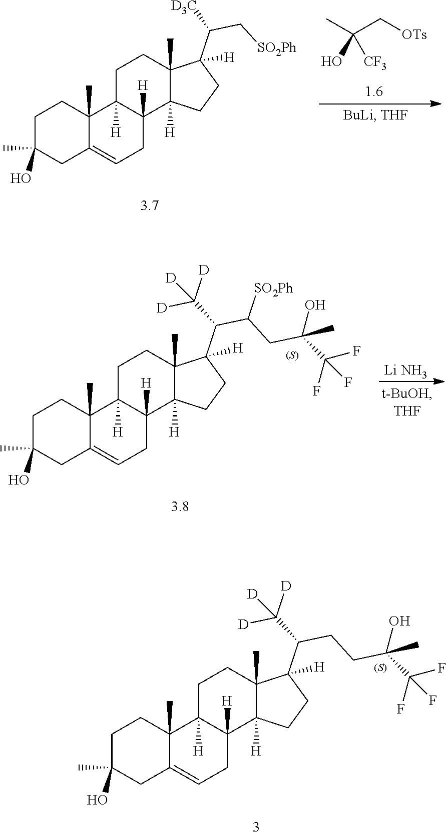

3.1 was prepared as described in WO2013/36835.

### Synthesis of 3.2

To a suspension of Mg (5.11 g, 212 mmol) and 12 (34.7 mg, 0.137 mmol) in ether (20 mL) was added a solution of CD₃I (10 g, 68.9 mmol) in ether (80 mL) dropwise under N₂ at 25° C. and the internal temperature was raised to 35° C. After stirring at 35° C. for 2 h, the mixture was reacted with 3.1 (5 g, 15.7 mmol) in THF (60 mL) at 0° C. and the mixture was stirred for 3 h. The mixture was poured into saturated aqueous NH₄Cl (80 mL) at 0° C. and extracted with EtOAc (3×100 mL). The combined organic layers were dried over anhydrous Na₂SO₄, filtered, and concentrated to give 3.2 (5.5 g). ¹H NMR (400 MHz, CDCl₃) δ_{H} 5.33-5.27 (m, 1H), 3.82-3.62 (m, 1H), 2.47-2.37 (m, 1H), 2.14-1.68 (m, 7H), 1.57-1.13 (m, 14H), 1.11 (s, 3H), 1.03-1.01 (m, 3H), 0.79-0.66 (m, 3H).

### Synthesis of 3.3

To a solution of 3.2 (1.2 g) in DCM (20 mL) was added NaHCO₃ (4.5 g, 53.5 mmol) and Dess-martin periodinane (4.5 g, 10.7 mmol) at 25° C. and the resulting mixture was stirred for 20 min. The mixture was quenched with saturated aqueous Na₂S₂O₃ (40 mL) and extracted with DCM (2×50 mL). The combined organic layers were washed with brine (2×10 mL), dried over anhydrous Na₂SO₄, filtered, and concentrated under vacuum. The residue was purified by silica gel chromatography (0˜25% EtOAc in PE) to give 3.3 (350 mg, 29.4%). ¹H NMR (400 MHz, CDCl₃) δ_{H} 5.38-5.29 (m, 1H), 2.56-2.49 (m, 1H), 2.45-2.40 (m, 1H), 1.76-1.53 (m, 19H), 1.12 (s, 3H), 1.01 (s, 3H), 0.63 (s, 3H).

### Synthesis of 3.4

To a suspension of MePPh₃Br (2.99 g, 8.37 mmol) in THF (15 mL) was added n-BuLi (2.98 mL, 7.47 mmol, 2.5 M) at 25° C. under N₂ and the resulting mixture was stirred for 30 min. 3.3 (1 g, 9.59 mmol) in THF (10 mL) was added over 1 min at 25° C. The reaction mixture was quenched with saturated aqueous NH₄Cl (20 ml) at 20° C. and the aqueous layer was extracted with EtOAc (3×20 mL). The combined organic layers were concentrated, and the residue was purified by silica gel chromatography to give 3.4 (580 mg, 58.5%). HRMS showed D %=98.4%; ¹H NMR (400 MHz, CDCl₃) δ_{H} 5.36-5.25 (m, 1H), 4.89-4.61 (m, 2H), 2.48-2.38 (m, 1H), 2.12-1.66 (m, 9H), 1.57-1.13 (m, 10H), 1.12 (s, 3H), 1.02 (s, 3H), 1.00-0.92 (m, 1H), 0.59 (s, 3H).

### Synthesis of 3.5

To a solution of 3.4 (1.15 g, 3.46 mmol) in THE (15 mL) was added 9-BBN (1.68 g, 6.92 mmol) and the resulting mixture was stirred at 25° C. for 16 h. Ethanol (2.43 mL, 41.5 mmol) was added, followed by aqueous NaOH (8.30 mL, 5.0 M, 41.5 mmol). H₂O₂ (4.56 mL, 10 M, 5.01 g) was added, and the reaction mixture was stirred at 25° C. for 1 h. The mixture was quenched with Na₂S₂O₃ (50 mL, sat. aq.) and extracted with EtOAc (3×30 mL). The combined organic layers were washed with saturated aqueous Na₂S₂O₃ (20 mL) and brine (20 mL), dried over anhydrous Na₂SO₄, filtered, concentrated, and purified by silica gel chromatography (0-30% EtOAc in PE) to give 3.5 (1.2 g). ¹H NMR (400 MHz, CDCl₃) δ_{H} 5.32-5.28 (m, 1H), 3.64 (dd, J=3.2, 10.4 Hz, 1H), 3.37 (dd, J=6.8, 10.4 Hz, 1H), 2.46-2.39 (m, 1H), 2.04-1.93 (m, 3H), 1.72-1.17 (m, 19H), 1.11 (s, 3H), 1.02 (s, 3H), 0.70 (s, 3H).

### Synthesis of 3.6

To a solution of 3.5 (1.2 g, 3.43 mmol) in DCM (20 mL) was added 1-methyl-1H-imidazole (1.12 g, 13.7 mmol) and TsCl (1.3 g, 6.86 mmol) at 20° C. and the resulting mixture was stirred for 1 h. The mixture was poured into H₂O (20 mL) and extracted with DCM (3×10 mL). The combined organic layers were washed with saturated HCl (1 M, 20 mL) and NaHCO₃ (sat. aq. 20 mL), dried over anhydrous Na₂SO₄, filtered, and concentrated to give 3.6 (1.5 g). ¹H NMR (400 MHz, CDCl₃) δ_{H} 7.78 (d, J=8.4 Hz, 2H), 7.34 (d, J=8.0 Hz, 2H), 5.30-5.27 (m, 1H), 4.17-3.72 (m, 2H), 2.45 (s, 3H), 2.44-2.37 (m, 1H), 2.02-1.77 (m, 4H), 1.69-1.30 (m, 13H), 1.28-1.12 (m, 4H), 1.11 (s, 3H), 1.00 (s, 3H), 0.69-0.60 (m, 3H).

### Synthesis of 3.7

To a solution of 3.6 (1.5 g, 2.97 mmol) in DMF (10 mL) was added KI (2.45 g, 14.8 mmol) and the resulting mixture was stirred at 60° C. for 1 h. PhSO₂Na (1.46 g, 8.91 mmol) was added, and the mixture was stirred at 60° C. for 12 h. The mixture was cooled to 20° C. and quenched with 10% aqueous NH₄Cl (50 mL). The mixture was extracted with EtOAc (3×80 mL) and the combined organic layers were washed with 3% aqueous LiCl (2×50 mL), dried over anhydrous Na₂SO₄, filtered, concentrated, and purified by silica gel chromatography (0-20% EA in PE) to give 3.7 (1.2 g). ¹H NMR (400 MHz, CDCl₃) δ_{H} 7.96-7.89 (m, 2H), 7.67-7.62 (m, 1H), 7.59-7.54 (m, 2H), 5.30-5.26 (m, 1H), 3.17-3.09 (m, 1H), 2.89-2.78 (m, 1H), 2.45-2.35 (m, 1H), 2.14-1.88 (m, 4H), 1.78-1.65 (m, 3H), 1.55-1.14 (m, 10H), 1.10 (s, 3H), 1.07-1.00 (m, 2H), 0.99 (s, 3H), 0.96-0.74 (m, 2H), 0.65 (s, 3H).

### Synthesis of 3.8

3.8 is prepared from 3.7 using a procedure as described for 1.7 of Example 1.

### Synthesis of 3

Li (0.35 g, 49.8 mmol) was added to liquid NH₃ (50 mL) at −70° C. under N₂ and the resulting mixture was stirred for 1 h. A solution of 3.8 (860 mg, 1.43 mmol) and t-BuOH (211 mg, 2.86 mmol) in THE (40 mL) was added and the reaction was stirred at −70° C. for 1 h. The reaction was quenched with NH₄Cl (5 g) at −70° C. and the mixture was warmed to 25° C. The reaction mixture was diluted with water (50 mL) and extracted with EtOAc (3×50 mL). The combined organic layers were dried over anhydrous Na₂SO₄, filtered, and concentrated. The mixture was purified by silica gel chromatography (15-25% EtOAc in PE) and lyophilized to give 3 (224.2 mg, 34%). ¹H NMR (400 MHz, CDCl₃) δ_{H} 5.37-5.25 (m, 1H), 2.49-2.36 (m, 1H), 2.05-1.54 (m, 13H), 1.51-1.34 (m, 7H), 1.32 (s, 3H), 1.28-1.12 (m, 4H), 1.11 (s, 3H), 1.10-1.05 (m, 1H), 1.01 (s, 3H), 0.99-0.94 (m, 1H), 0.68 (s, 3H). ¹⁹F NMR (400 MHz, CDCl₃) δ_{F} −83.13 (s). LC-ELSD/MS 30-90AB_2 min_E, purity >99%; MS ESI calcd. for C₂₇H₃₉D₃F₃O [M+H−H₂O]⁺ 442.4, found 442.4.

### Example 5: Synthesis of (3S,8S,9S,10R,13R,14S,17R)-3,10,13-trimethyl-17-((2R,5S)-6,6,6-trifluoro-5-hydroxy-5-methylhexan-2-yl)-2,3,4,7,8,9,10,11,12,13,14,15,16,17-tetradecahydro-1H-cyclopenta[a]phenanthren-7,7-d2-3-ol (4)

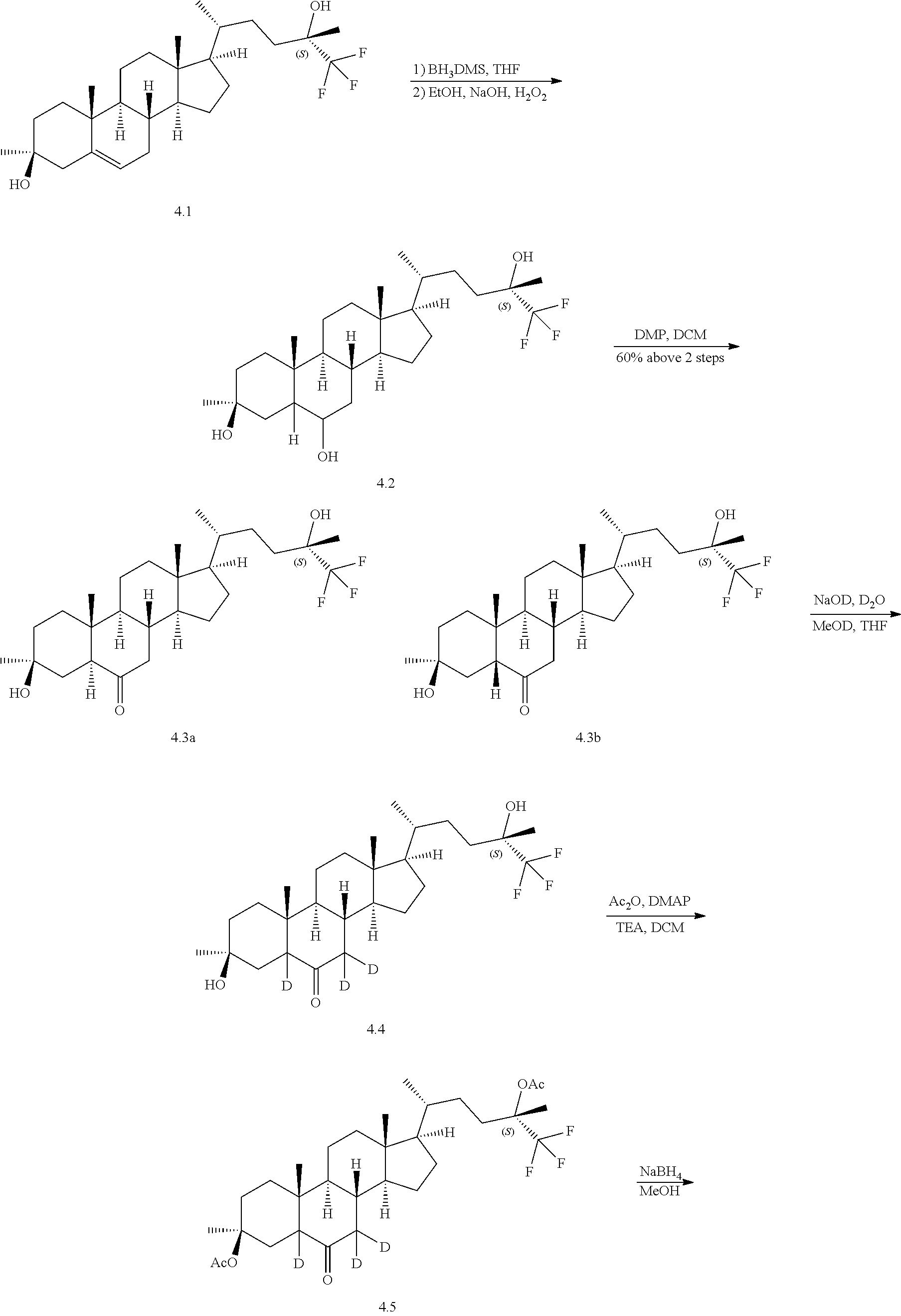

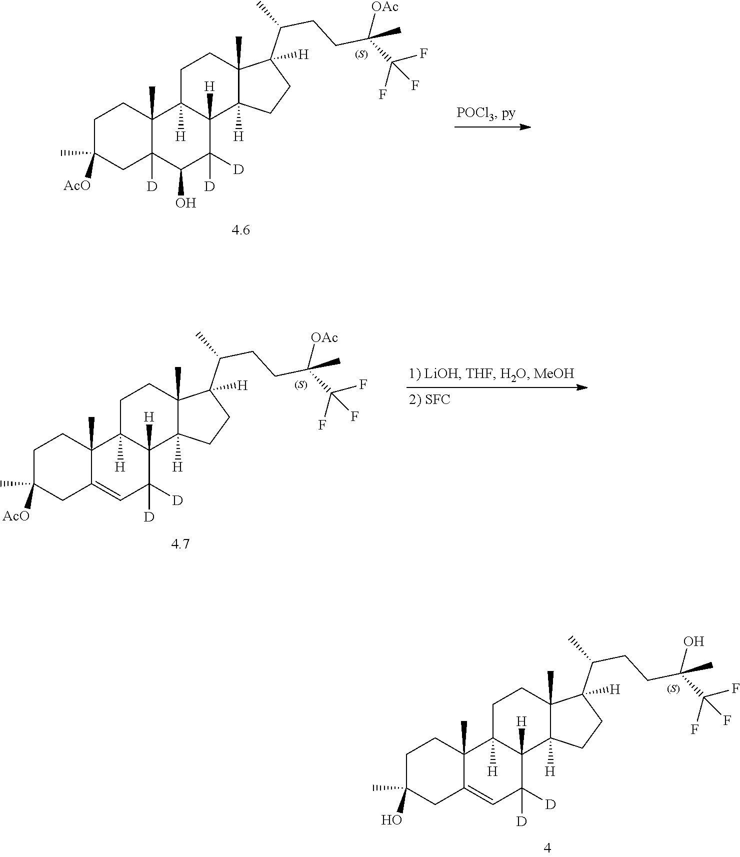

4.1 was prepared as described in WO2018075699 A1,

### Synthesis of 4.2

To a solution of 4.1 (500 mg, 1.09 mmol) in THE (5 mL) was added BH₃·DMS (1.09 mL, 10 M, 10.9 mmol) and the resulting mixture was stirred at 25° C. for 16 h. Ethanol (1.92 mL, 32.6 mmol) was added, followed by aqueous NaOH (6.52 mL, 5.0 M, 32.6 mmol). H₂O₂ (3.26 mL, 10 M, 32.6 mmol) was added at 25° C. and the mixture was stirred at 60° C. for 1 h. The mixture was quenched with saturated aqueous Na₂S₂O₃ (40 mL) and extracted with EtOAc (3×40 mL). The combined organic layers were washed with saturated aqueous Na₂S₂O₃ (2×40 mL), dried over anhydrous Na₂SO₄, filtered, and concentrated to give 4.2 (560 mg, C-5 and C-6 isomer mixture).

### Synthesis of 4.3a and 4.3b

To a solution of 4.2 (500 mg, 0.1053 mmol) in DCM (10 mL) was added Dess-martin periodinane (1.78 g, 4.20 mmol) at 25° C. and the resulting mixture was stirred for 1 h. The mixture was quenched with saturated aqueous NaHCO₃ (20 mL). The DCM phase was separated and washed with saturated aqueous NaHCO₃/Na₂S₂O₃ (1:1, 2×20 mL), brine (20 mL), dried over anhydrous Na₂SO₄, filtered, and concentrated under vacuum. The mixture was purified by silica gel chromatography (15-30% EtOAc in PE) to give 4.3a (280 mg, 56.4%) and 4.3b (40 mg, 8.06%). 4.3a—¹H NMR (400 MHz, CDCl₃) δ_{H} 2.42 (dd, J=3.6, 13.2 Hz, 1H), 2.16 (dd, J=3.6, 13.6 Hz, 1H), 2.12-1.56 (m, 11H), 1.55-1.30 (m, 11H), 1.28 (s, 3H), 1.23 (s, 3H), 1.20-1.00 (m, 4H), 0.92 (d, J=6.4 Hz, 3H), 0.84 (s, 3H), 0.63 (s, 3H). ¹⁹F NMR (400 MHz, CDCl₃) δ_{F} −82.98.

### Synthesis of 4.4

To a solution of 4.3a and 4.3b (290 mg, 0.6135 mmol) in THE (6 mL) was added NaOD (4.2 mL, 0.1 M in D₂O, 0.42 mmol) and CD₃OD (0.5 mL) at 50° C. under N₂ and the resulting mixture was stirred for 16 h. The reaction mixture was adjusted to pH=7 with AcOD (0.1 M in D₂O) and NaCl (200 mg) and EtOAc (10 mL) were added. The mixture was stirred for 5 min and the organic layer was separated, dried over anhydrous Na₂SO₄, filtered, and concentrated in vacuo to give 4.4 (280 mg, C-5 isomer mixture).

### Synthesis of 4.5

To a solution of 4.4 (280 mg, 0.8737 mmol) in DCM (4 mL) was added DMAP (106 mg, 0.8737 mmol), TEA (175 mg, 1.74 mmol) and acetyl acetate (177 mg, 0.163 mL, 1.74 mmol) and the resulting mixture was stirred at 25° C. for 16 h. The residue was poured into 10% aqueous NaHCO₃ (10 mL) and stirred for 10 min. The aqueous phase was extracted with DCM (2×5 mL) and the combined organic layers were washed with brine (2×5 mL), dried over anhydrous Na₂SO₄, filtered, and concentrated to give 4.5 (320 mg, C-5 isomer mixture).

### Synthesis of 4.6

To a solution of 4.5 (320 mg, 0.5717 mmol) in MeOH (10 mL) was added NaBH₄ (216 mg, 5.71 mmol) at 0° C. under N₂ and the resulting mixture was stirred at 25° C. for 0.5 h. The reaction mixture was quenched with saturated aqueous NH₄Cl (50 mL) at 20° C. and the aqueous layer was extracted with EtOAc (2×50 mL). The combined organic layers were concentrated to give 4.6 (280 mg, above 3 steps 81.2%, C-5 isomer mixture).

### Synthesis of 4.7

To a solution of 4.6 (200 mg, 0.3560 mmol) in pyridine (4 mL) was added POCl₃ (0.496 mL, 5.33 mmol) at 0° C. under N₂ and the resulting mixture was stirred at 40° C. for 16 h. The mixture was slowly quenched with H₂O (20 mL) at 0° C. and extracted with EtOAc (2×20 mL). The combined organic layers were washed with brine (20 mL), dried over anhydrous Na₂SO₄, filtered, and concentrated to give 4.7 (200 mg), which was used directly in the next step.

### Synthesis of 4

To a solution of 4.7 (260 mg, 0.4790 mmol) in MeOH (2 mL) and THE (2 mL) was added LiOH·H₂O (499 mg, 11.9 mmol) at 25° C. and the resulting mixture was stirred for 18 h. The mixture was poured into water (10 mL), filtered, dried over anhydrous Na₂SO₄, filtered, and concentrated. The mixture was purified by silica gel chromatography (0-12% EtOAc in PE), separated by SFC (column: DAICEL CHIRALPAK AD (250 mm*30 mm, 10 um); Condition: 0.1% NH₃·H₂O EtOH; Begin B: 20%; End B: 20%; Gradient Time (min): 45; Flow Rate (ml/min): 70; injections: 120) and lyophilized to give 4 (30.2 mg, 13.7%, DO: 1.6%, D1: 6.2%, D2: 91.4%). ¹HNMR (400 MHz, CDCl₃) δ_{H} 5.36-5.23 (m, 1H), 2.48-2.36 (m, 1H), 2.06-1.94 (m, 2H), 1.91-1.56 (m, 8H), 1.54-1.33 (m, 7H), 1.32 (s, 3H), 1.31-1.12 (m, 5H), 1.11 (s, 3H), 1.10-1.04 (m, 1H), 1.01 (s, 3H), 1.00-0.96 (m, 1H), 0.94 (d, J=6.8 Hz, 3H), 0.68 (s, 3H). ¹⁹F NMR (400 MHz, CDCl₃) δ_{F} −83.13. LC-ELSD/MS 30-90AB_2 min_E, purity >99%, MS ESI calcd. for C₂₇H₄₀D₂F₃O [M−H₂O+H]⁺ 441.3, found 441.3.

### Example 6

### Assay Methods

Compounds of the present disclosure can be evaluated using various in vitro and in vivo assays described in the literature; examples of which are described below.

The following examples are offered to illustrate the biological activity of the compounds, pharmaceutical compositions, and methods provided herein and are not to be construed in any way as limiting the scope thereof.

### NMDA Modulation

### Whole-Cell Patch Clamp of Mammalian Cells (Syncropatch 384i (Nanion Technologies))

Whole-cell patch clamp electrophysiology is used to investigate the effects of compounds on GluN1/GluN2A N-methyl-D-aspartate (NMDA) glutamate receptors expressed in mammalian cells.

### Cell Culture

Human embryonic kidney (HEK293) cells expressing the recombinant human GluN1/GluN2A receptor under the control of a tetracycline-inducible expression system are used. Cells are maintained in Dulbecco's Modified Eagle's Medium (DMEM) with 1% glutamine, 10% Tetracycline System Approved fetal bovine serum (FBS), and NMDA inhibitors. Cells are cultured in a humidified 5% CO₂ incubator at 37° C. Cells are induced with 50 ng/ml tetracycline 18-24 hours prior to testing. A cocktail of channel blockers is added to the media to prevent cell death.

Cells are 60-80% confluent at the time of harvest. Cells are detached from the flask TrypLE Enzyme Express (Thermo Fisher) at 37° C., and then cold extracellular solution is added to the flask, followed by incubation at 4° C. and then trituration of the cells. The cell solution is then placed on a shaker on the Syncropatch platform at 10° C. with a shake speed of a minimum 200 rpm for at least 30 minutes prior to testing.

### Solutions

Extracellular (EC) solution (in mM)—140 NaCl, 4 KCl, 5 CaCl₂, 10 HEPES, 5 Glucose, pH 7.4 with NaOH. All external solutions contain 0.2% DMSO and 0.01% Kolliphor® EL (C₅₁₃₅, Sigma) throughout the experiment.

Intracellular (IC) solution (in mM)—120 CsF, 10 mM EGTA, 10 NaCl, 10 HEPES, 4 NaATP, 2 MgCl₂, pH 7.2 with CsOH

### Plate Preparation

First, the DMSO Master Plate is prepared, in which the DMSO stock of each test compound is serially diluted in 100% DMSO to 1000× the final assay concentration. Immediately prior to assay testing, the compounds are diluted 1:100 by transferring solutions from the DMSO Mast Plate to the Intermediate Plate pre-filled with EC solution using a Biomek FX automated liquid handling system. Compounds are then diluted further with extracellular solution into two pre-incubation plates—1) compound from the Intermediate Plate are mixed with EC solution at a 1:5 dilution to create a 2× Compound Pre-Incubation Plate which is diluted a further 1:2 when added to the Syncropatch for the initial compound pre-incubation. And 2) compound from the Intermediate plate is mixed with EC solution at a 1:10 Dilution to create the 1× Compound Pre-Incubation Plate, which is used for subsequent compound pre-incubations. Finally, a Compound+Agonist Plate is prepared by mixing compound from the 2× Compound Pre-Incubation Plate with 2× concentration of co-agonists in a 1:1 ratio.

### Voltage Protocol

Each experiment sequentially includes cell catching, sealing, whole-cell formation, liquid application, recording and data acquisition. Whole-cell patch clamp recordings are performed using multi-hole high resistance chips on the SyncroPatch 384i (Nanion Technologies). A steady-state voltage pulse at −80 mV is applied throughout the assay and a 10 second recording window is triggered around 1 second prior to application of co-agonists. Currents are leak corrected and sampled at 5 kHz.

### Application Protocol

A fast application is used, in which 5 μl co-agonist is rapidly applied at a speed of 30 μl/s and then rapidly removed from the well. To test for modulator activity, the co-agonists glutamate (1 μM) and glycine (100 μM) are applied three times to show activation reproducibility, followed by 120 seconds pre-incubation of test compound alone. The test compound s then re-applied in the presence of co-agonists two times. This is followed by application of the agonist alone, although test compound may not be fully washed from the well prior to this step. Finally, a saturating concentration of co-agonists (glutamate 100 μM/glycine 100 μM) is applied.

### Analysis

The voltage protocol generation data collection and analysis is performed on PatchControl1384 and DataControl1384. For each concentration tested, the fold peak amplitude increase is generated using the following equation: (Icomp/Icontrol)-1, where Icomp is the mean current peak amplitude in the presence of the test compound and co-agonists from the two compound/co-agonist applications, and Icontrol is the mean current peak amplitude in the presence of agonist alone from the second and third co-agonist applications. Test compounds are evaluated at 8 concentrations, with at least 2 replicate wells per concentration. The effect of all concentrations is then fitted with a four-parameter logistic curve fit using least squares regression and the EC50 and Emax are calculated. (GraphPad Prism). For compounds that are tested on multiple assays, the geometric mean of the EC50 and arithmetic mean of the Emax is calculated using the parameters derived from the curve fits on each individual assay.

### EQUIVALENTS

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

### Other Embodiments

The disclosure also includes the following numbered paragraphs:
1. A compound of Formula (I):

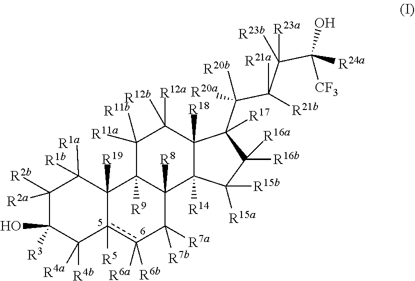

or a pharmaceutically acceptable salt thereof;
wherein:
each of R³, R¹⁸, R¹⁹, R^{20a} and R^{24a} is independently C₁₋₃alkyl optionally substituted with 1 or more independent occurrences of deuterium;
each of R^{1a}, R^{1b}, R^{2a}, R^{2b}, R^{4a}, R^{4b}, R⁵, R^{6a}, R^{6b}, R^{7a}, R⁸, R⁹, R^{11a}, R^{11b}, R^{12a}, R^{12b}, R¹⁴, R^{15a}, R^{15b}, R^{16a}, R^{16b}, R¹⁷, R^{20b}, R^{21a}, R^{21b}, R^{23a}, and, R^{23b} is independently selected from a group consisting of hydrogen and deuterium;
provided that at least one of R³, R¹⁸, R¹⁹, R^{20a} and R^{24a} is substituted with 1 or more independent occurrences of deuterium, or at least one of R^{1a}, R^{1b}, R^{2a}, R^{2b}, R^{4a}, R^{4b}, R⁵, R^{6a}, R^{6b}, R^{7a}, R^{7b}, R⁸, R⁹, R^{11a}, R^{11b}, R^{12a}, R^{12b}, R¹⁴, R^{15a}, R^{15b}, R^{16a}, R^{16b}, R¹⁷, R^{20b}, R^{21a}, R^{21b}, R^{23a}, and R^{23b} is deuterium;
[symbol] represents a single or double bond; provided if the bond between C5 and C6 is a single bond, then R⁵ is in the alpha configuration.
2. The compound of paragraph 1, wherein the compound of Formula (I) is a compound of Formula (Ia):

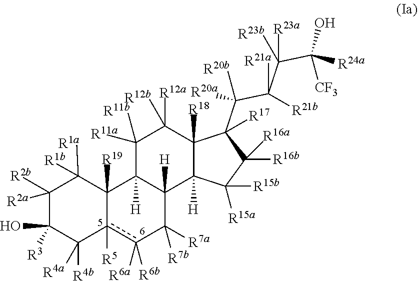

or a pharmaceutically acceptable salt thereof;
wherein:
each of R³, R¹⁸, R¹⁹, R^{20a} and R^{24a} is independently C₁₋₃alkyl optionally substituted with 1 or more independent occurrences of deuterium;
each of R^{1a}, R^{1b}, R^{2a}, R^{2b}, R^{4a}, R^{4b}, R⁵, R^{6a}, R^{6b}, R^{7a}, R^{7b}, R^{11a}, R^{11b}, R^{12a}, R^{12b}, R^{15a}, R^{15b}, R^{16a}, R^{16b}, R¹⁷, R^{20b}, R^{21a}, R^{21b}, R^{23a}, and R^{23b} is independently selected from a group consisting of hydrogen and deuterium;
provided that at least one of R³, R¹⁸, R¹⁹, R^{20a} and R^{24a} is substituted with 1 or more independent occurrences of deuterium, or at least one of R^{1a}, R^{1b}, R^{2a}, R^{2b}, R^{4a}, R^{4b}, R⁵, R^{6a}, R^{6b}, R^{7a}, R^{7b}, R^{11a}, R^{11b}, R^{12a}, R^{12b}, R^{15a}, R^{15b}, R^{16a}, R^{16b}, R¹⁷, R^{20b}, R^{21a}, R^{21b}, R^{23a}, and R^{23b} is deuterium;
[symbol] represents a single or double bond; provided if the bond between C5 and C6 is a single bond, then R⁵ is in the alpha configuration.
3. The compound of paragraph 2, wherein the compound of Formula (Ia) is a compound of Formula (II):

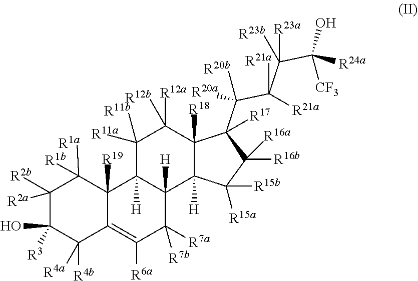

or a pharmaceutically acceptable salt thereof:
4. The compound of paragraph 2, wherein the compound of Formula (Ia) is a compound of Formula (III):

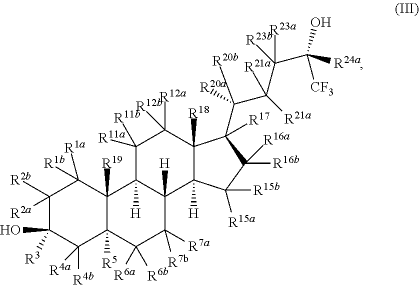

or a pharmaceutically acceptable salt thereof;
5. The compound or pharmaceutically acceptable salt thereof according to any one of paragraphs 1-3, wherein at least one of R^{3a}, R¹⁸, R¹⁹, R^{20a}, or R^{24a} is C₁₋₃ alkyl substituted with 1 or more independent occurrences of deuterium, or at least one of R^{2a}, R^{2b}, R^{4a}, R^{4b}, R⁵, R^{6a}, R^{7a}, R^{7b}, R^{11a}, R^{11b}, R^{15a} and R^{15b} is deuterium.
6. The compound or pharmaceutically acceptable salt thereof according to any one of paragraphs 1-4, wherein at least one of R^{3a}, R^{3b}, R¹⁹, R^{20a}, or R^{24a} is C₁₋₃ alkyl substituted with 1 or more independent occurrences of deuterium.
7. The compound or pharmaceutically acceptable salt thereof according to any one of paragraphs 1-4, wherein at least one of R^{2a}, R^{2b}, R^{4a}, R^{4b}, R⁵, R^{6a}, R^{7a}, R^{7b}, R^{11a}, R^{11b}, R^{15a} and R^{15b} is deuterium.
8. The compound or a pharmaceutically acceptable salt according to any one of paragraphs 1-7, wherein each of R^{1a} and R^{1b} is independently deuterium.
9. The compound or a pharmaceutically acceptable salt according to any one of paragraphs 1-7, wherein each of R^{1a} and R^{1b} is independently hydrogen.
10. The compound or a pharmaceutically acceptable salt according to any one of paragraphs 1-9, wherein each of R^{2a} and R^{2b} is independently deuterium.
11. The compound or a pharmaceutically acceptable salt according to any one of paragraphs 1-9, wherein each of R^{2a} and R^{2b} is independently hydrogen.
12. The compound or pharmaceutically acceptable salt thereof according to any one of paragraphs 1-11, wherein R³ is —CH₃.
13. The compound or pharmaceutically acceptable salt thereof according to any one of paragraphs 1-11, wherein R³ is C₁₋₃alkyl substituted with 1 or more independent occurrences of deuterium.
14. The compound or pharmaceutically acceptable salt thereof according to paragraph 13, wherein R³ is methyl substituted with 1-3 independent occurrences of deuterium.
15. The compound or pharmaceutically acceptable salt thereof according to paragraph 14, wherein R³ is —CD₃.
16. The compound or a pharmaceutically acceptable salt according to any one of paragraphs 1-15, wherein each of R^{4a} and R^{4b} is independently deuterium.
17. The compound or a pharmaceutically acceptable salt according to any one of paragraphs 1-15, wherein each of R^{4a} and R^{4b} is independently hydrogen.
18. The compound or a pharmaceutically acceptable salt according to any one of paragraphs 1-17, wherein R⁵ is deuterium.
19. The compound or a pharmaceutically acceptable salt according to any one of paragraphs 1-17, wherein R⁵ is hydrogen.
20. The compound or a pharmaceutically acceptable salt according to any one of paragraphs 1-17, wherein each of R⁵ and R^{6a} is independently deuterium.
21. The compound or a pharmaceutically acceptable salt according to any one of paragraphs 1-17, wherein each of R⁵ and R^{6a} is independently hydrogen.
22. The compound or a pharmaceutically acceptable salt according to any one of paragraphs 1-19, wherein each of R^{6a} and R^{6b} is independently deuterium.
23. The compound or a pharmaceutically acceptable salt according to any one of paragraphs 1-19, wherein each of R^{6a} and R^{6b} is independently hydrogen.
24. The compound or a pharmaceutically acceptable salt according to any one of paragraphs 1-23, wherein each of R^{7a} or R^{7b} is independently deuterium.
25. The compound or a pharmaceutically acceptable salt according to any one of paragraphs 1-23, wherein each of R^{7a} or R^{7b} is independently hydrogen.
26. The compound or a pharmaceutically acceptable salt according to any one of paragraphs 1-25, wherein each of R^{11a} and R^{11b} is independently deuterium.
27. The compound or a pharmaceutically acceptable salt according to any one of paragraphs 1-25, wherein each of R^{11a} and R^{11b} is independently hydrogen.
28. The compound or a pharmaceutically acceptable salt according to any one of paragraphs 1-27, wherein each of R^{12a} and R^{12b} is independently deuterium.
29. The compound or a pharmaceutically acceptable salt according to any one of paragraphs 1-27, wherein each of R^{12a} and R^{12b} is independently hydrogen.
30. The compound or a pharmaceutically acceptable salt according to any one of paragraphs 1-29, wherein each of R^{15a} and R^{15b} is independently deuterium.
31. The compound or a pharmaceutically acceptable salt according to any one of paragraphs 1-29, wherein each of R^{15a} and R^{15b} is independently hydrogen.
32. The compound or a pharmaceutically acceptable salt according to any one of paragraphs 1-31, wherein each of R^{16a} and R^{16b} is independently deuterium.
33. The compound or a pharmaceutically acceptable salt according to any one of paragraphs 1-31, wherein each of R^{16a} and R^{16b} is independently hydrogen.
34. The compound or a pharmaceutically acceptable salt according to any one of paragraphs 1-33, wherein R¹⁷ is deuterium.
35. The compound or a pharmaceutically acceptable salt according to any one of paragraphs 1-33, wherein R¹⁷ is hydrogen.
36. The compound or pharmaceutically acceptable salt thereof according to any one of paragraphs 1-35, wherein R¹⁸ is C₁₋₃alkyl substituted with 1 or more independent occurrences of deuterium.
37. The compound or pharmaceutically acceptable salt thereof according to paragraph 36, wherein R¹⁸ is methyl substituted with 1-3 independent occurrences of deuterium.
38. The compound or pharmaceutically acceptable salt thereof according to paragraph 37, wherein R¹⁸ is —CD₃.
39. The compound or pharmaceutically acceptable salt thereof according to any one of paragraphs 1-35, wherein R¹⁸ is —CH₃.
40. The compound or pharmaceutically acceptable salt thereof according to any one of paragraphs 1-39, wherein R¹⁹ is C₁₋₃alkyl substituted with 1 or more independent occurrences of deuterium.
41. The compound or pharmaceutically acceptable salt thereof according to paragraph 40, wherein R¹⁹ is methyl substituted with 1-3 independent occurrences of deuterium.
42. The compound or pharmaceutically acceptable salt thereof according to paragraph 41, wherein R¹⁹ is —CD₃.
43. The compound or pharmaceutically acceptable salt thereof according to any one of paragraphs 1-39, wherein R¹⁹ is —CH₃.
44. The compound or a pharmaceutically acceptable salt according to any one of paragraphs 1-43, wherein R^{20a} is C₁₋₃alkyl substituted with 1 or more independent occurrences of deuterium.
45. The compound or pharmaceutically acceptable salt thereof according to paragraph 44, wherein R^{20a} is methyl substituted with 1-3 independent occurrences of deuterium.
46. The compound or pharmaceutically acceptable salt thereof according to paragraph 45, wherein R^{20a} is —CD₃.
47. The compound or pharmaceutically acceptable salt thereof according to any one of paragraphs 1-43, wherein R^{20a} is —CH₃.
48. The compound or a pharmaceutically acceptable salt according to any one of paragraphs 1-47, wherein R^{20b} is deuterium.
49. The compound or a pharmaceutically acceptable salt according to any one of paragraphs 1-47, wherein R^{20b} is hydrogen.
50. The compound or a pharmaceutically acceptable salt according to any one of paragraphs 1-49, wherein each of R^{21a} and R^{21b} is independently deuterium.
51. The compound or a pharmaceutically acceptable salt according to any one of paragraphs 1-49, wherein each of R^{21a} and R^{21b} is independently hydrogen.
52. The compound or a pharmaceutically acceptable salt according to any one of paragraphs 1-51, wherein each of R^{23a} and R^{23b} is independently deuterium.
53. The compound or a pharmaceutically acceptable salt according to any one of paragraphs 1-51, wherein each of R^{23a} and R^{23b} is independently hydrogen.
54. The compound or a pharmaceutically acceptable salt according to any one of paragraphs 1-53, wherein R^{24a} is C₁₋₃alkyl substituted with 1 or more independent occurrences of deuterium.
55. The compound or pharmaceutically acceptable salt thereof according to paragraph 54, wherein R^{24a} is methyl substituted with 1-3 independent occurrences of deuterium.
56. The compound or pharmaceutically acceptable salt thereof according to paragraph 55, wherein R^{24a} is —CD₃.
57. The compound or pharmaceutically acceptable salt thereof according to any one of paragraphs 1-53, wherein R^{24a} is —CH₃.
58. The compound of paragraph 1 wherein the compound of formula (I) is a compound of formula (IV)

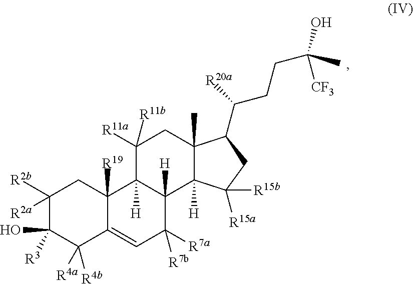

or a pharmaceutically acceptable salt thereof.
59. The compound or pharmaceutically acceptable salt thereof according to paragraph 58, wherein the compound of Formula (IV) is selected from the group consisting of any one of compounds A-J;
   or a pharmaceutically acceptable salt thereof, wherein any atom not labeled as deuterium is present at its natural isotopic abundance.
60. The compound of paragraph 1 wherein the compound of formula (I) is a compound of formula (V)

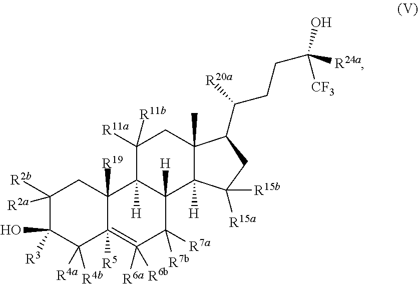

or a pharmaceutically acceptable salt thereof.
61. The compound of paragraph 60, wherein the compound of Formula (V) is selected from the group consisting of any one of compounds K-X;
   or a pharmaceutically acceptable salt thereof, wherein any atom not labeled as deuterium is present at its natural isotopic abundance.
62 A pharmaceutical composition comprising a compound according to any one of paragraphs 1-61, and a pharmaceutically acceptable carrier.
63. A method for treating a CNS-related condition in a subject, comprising administering to the subject an effective amount of a compound according to any one of paragraphs 1-61, or a pharmaceutical composition according to paragraph 62.
64. The method according to paragraph 63, wherein the CNS-related condition is selected from the group consisting of an adjustment disorder, anxiety disorder (including obsessive-compulsive disorder, post-traumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.
65. A method of inducing sedation or anesthesia in a subject, comprising administering to the subject an effective amount of a compound according to any one of paragraphs 1-61, or a pharmaceutical composition according to paragraph 62.
66. A compound according to any one of paragraphs 1-61, or a pharmaceutical composition according to paragraph 62, for use in treating a CNS-related condition in a subject.
67. The compound or pharmaceutical composition for use according to paragraph 66, wherein the CNS-related condition is selected from the group consisting of an adjustment disorder, anxiety disorder (including obsessive-compulsive disorder, post-traumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.
68. A compound according to any one of paragraphs 1-61, or a pharmaceutical composition according to paragraph 62, for use inducing sedation or anesthesia in a subject.
69. Use of a compound according to any one of paragraphs 1-61, or a pharmaceutical composition according to paragraph 62, for the manufacture of a medicament for treating a CNS-related condition in a subject.
70. The use according to paragraph 69, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.
71. A compound of Formula (I):

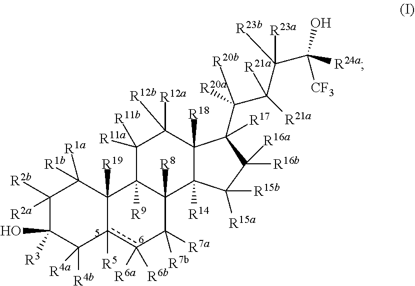

wherein:
each of R³, R¹⁸, R¹⁹, R^{20a} and R^{24a} is independently C₁₋₃alkyl optionally substituted with 1 or more independent occurrences of deuterium;
each of R^{1a}, R^{1b}, R^{2a}, R^{2b}, R^{4a}, R^{4b}, R⁵, R^{6a}, R^{6b}, R^{7a}, R^{7b}, R⁸, R⁹, R^{11a}, R^{11b}, R^{12a}, R^{12b}, R¹⁴, R^{15a}, R^{15b}, R^{16a}, R^{16b}, R¹⁷, R^{20b}, R²¹, R^{21b}, R^{23a}, and, R^{23b} is independently selected from a group consisting of hydrogen and deuterium;
provided that at least one of R³, R¹⁸, R¹⁹, R^{20a} and R^{24a} is substituted with 1 or more independent occurrences of deuterium, or at least one of R^{1a}, R^{1b}, R^{2a}, R^{2b}, R^{4a}, R^{4b}, R⁵, R^{6a}, R^{6b}, R^{7a}, R^{7b}, R⁸, R⁹, R^{11a}, R^{11b}, R^{12a}, R^{12b}, R¹⁴, R^{15a}, R^{15b}, R^{16a}, R^{16b}, R¹⁷, R^{20b}, R^{21a}, R^{21b}, R^{23a}, and R^{23b} is deuterium;
[symbol] represents a single or double bond; provided if the bond between C5 and C6 is a single bond, then R⁵ is in the alpha configuration.
72. The compound of paragraph 71, wherein the compound of Formula (I) is a compound of Formula (Ia):

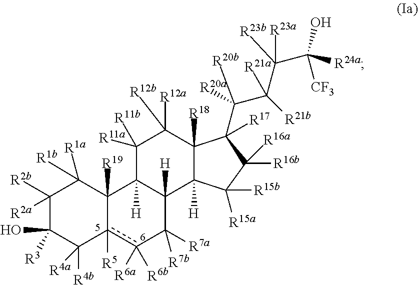

wherein:
each of R³, R¹⁸, R¹⁹, R^{20a} and R^{24a} is independently C₁₋₃alkyl optionally substituted with 1 or more independent occurrences of deuterium;
each of R^{1a}, R^{1b}, R^{2a}, R^{2b}, R^{4a}, R^{4b}, R⁵, R^{6a}, R^{6b}, R^{7a}, R^{7b}, R^{11a}, R^{11b}, R^{12a}, R^{12b}, R^{15a}, R^{15b}, R^{16a}, R^{16b}, R¹⁷, R^{20b}, R^{21a}, R^{21b}, R^{23a}, and R^{23b} is independently selected from a group consisting of hydrogen and deuterium;
provided that at least one of R³, R¹⁸, R¹⁹, R^{20a} and R^{24a} is substituted with 1 or more independent occurrences of deuterium, or at least one of R^{1a}, R^{1b}, R^{2a}, R^{2b}, R^{4a}, R^{4b}, R⁵, R^{6a}, R^{6b}, R^{7a}, R^{7b}, R^{11a}, R^{11b}, R^{12a}, R^{12b}, R^{15a}, R^{15b}, R^{16a}, R^{16b}, R¹⁷, R^{20b}, R^{21a}, R^{21b}, R^{23a}, and R^{23b} is deuterium;
[symbol] represents a single or double bond; provided if the bond between C5 and C6 is a single bond, then R⁵ is in the alpha configuration.
73. The compound according to paragraph 72, wherein the compound of Formula (Ia) is a compound of Formula (II):

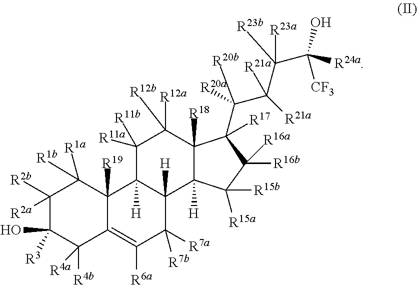

74. The compound according to paragraph 72, wherein the compound of Formula (Ia) is a compound of Formula (III):

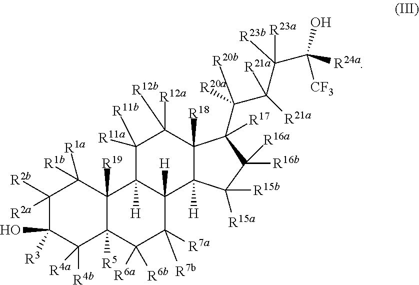

75. The compound according to any one of paragraphs 71-74, wherein at least one of R^{3a}, R¹⁸, R¹⁹, R^{20a}, or R^{24a} is C₁₋₃alkyl substituted with 1 or more independent occurrences of deuterium, or at least one of R^{2a}, R^{2b}, R^{4a}, R^{4b}, R⁵, R^{6a}, R^{7a}, R^{7b}, R^{11a}, R^{11b}, R^{15a} and R^{15b} is deuterium.
76. The compound according to any one of embodiments 71-74, wherein at least one of R^{3a}, R¹⁸, R¹⁹, R^{20a}, or R^{24a} is C₁₋₃ alkyl substituted with 1 or more independent occurrences of deuterium.
77. The compound according to any one of paragraphs 71-74, wherein at least one of R^{2a}, R^{2b}, R^{4a}, R^{4b}, R⁵, R^{6a}, R^{7a}, R^{7b}, R^{11a}, R^{11b}, R^{15a} and R^{15b} is deuterium.
78. The compound according to any one of paragraphs 71-77, wherein each of R^{1a} and R^{1b} is independently deuterium.
79. The compound according to any one of paragraphs 71-77, wherein each of R^{1a} and R^{1b} is independently hydrogen.
80. The compound according to any one of paragraphs 71-79, wherein each of R^{2a} and R^{2b} is independently deuterium.
81. The compound according to any one of paragraphs 71-79, wherein each of R^{2a} and R^{2b} is independently hydrogen.
82. The compound according to any one of paragraphs 71-81, wherein R³ is —CH₃.
83. The compound according to any one of paragraphs 71-81, wherein R³ is C₁₋₃alkyl substituted with 1 or more independent occurrences of deuterium.
84. The compound according to paragraph 83, wherein R³ is methyl substituted with 1-3 independent occurrences of deuterium.
85. The compound according to paragraph 84, wherein R³ is —CD₃.
86. The compound according to any one of paragraphs 71-85, wherein each of R^{4a} and R^{4b} is independently deuterium.
87. The compound according to any one of paragraphs 71-85, wherein each of R^{4a} and R^{4b} is independently hydrogen.
88. The compound according to any one of paragraphs 71-87, wherein R⁵ is deuterium.
89. The compound according to any one of paragraphs 71-87, wherein R⁵ is hydrogen.
90. The compound according to any one of paragraphs 71-87, wherein each of R⁵ and R^{6a} is independently deuterium.
91. The compound according to any one of paragraphs 71-87, wherein each of R⁵ and R^{6a} is independently hydrogen.
92. The compound according to any one of paragraphs 71-89, wherein each of R^{6a} and R^{6b} is independently deuterium.
93. The compound according to any one of paragraphs 71-89, wherein each of R^{6a} and R^{6b} is independently hydrogen.
94. The compound according to any one of paragraphs 71-93, wherein each of R^{7a} or R^{7b}, is independently deuterium.
95. The compound according to any one of paragraphs 71-93, wherein each of R^{7a} or R^{7b}, is independently hydrogen.
96. The compound according to any one of paragraphs 71-95, wherein each of R^{11a} and R^{11b} is independently deuterium.
97. The compound according to any one of paragraphs 71-95, wherein each of R^{11a} and R^{11b} is independently hydrogen.
98. The compound according to any one of paragraphs 71-97, wherein each of R^{12a} and R^{12b} is independently deuterium.
99. The compound according to any one of paragraphs 71-97, wherein each of R^{12a} and R^{12b} is independently hydrogen.
100. The compound according to any one of paragraphs 71-99, wherein each of R^{15a} and R^{15b} is independently deuterium.
101. The compound according to any one of paragraphs 71-99, wherein each of R^{15a} and R^{15b} is independently hydrogen.
102. The compound according to any one of paragraphs 71-101, wherein each of R^{16a} and R^{16b} is independently deuterium.
103. The compound according to any one of paragraphs 71-101, wherein each of R^{16a} and R^{16b} is independently hydrogen.
104. The compound according to any one of paragraphs 71-103, wherein R¹⁷ is deuterium.
105. The compound according to any one of paragraphs 71-103, wherein R¹⁷ is hydrogen.
106. The compound according to any one of paragraphs 71-105, wherein R¹⁸ is C₁₋₃alkyl substituted with 1 or more independent occurrences of deuterium.
107. The compound according to paragraph 106, wherein R¹⁸ is methyl substituted with 1-3 independent occurrences of deuterium.
108. The compound according to paragraph 107, wherein R¹⁸ is —CD₃.
109. The compound according to any one of paragraphs 71-105, wherein R¹⁸ is —CH₃.
110. The compound according to any one of paragraphs 71-109, wherein R¹⁹ is C₁₋₃alkyl substituted with 1 or more independent occurrences of deuterium.
111. The compound according to paragraph 110, wherein R¹⁹ is methyl substituted with 1-3 independent occurrences of deuterium.
112. The compound according to paragraph 111, wherein R¹⁹ is —CD₃.
113. The compound according to any one of paragraphs 71-109, wherein R¹⁹ is —CH₃.
114. The compound according to any one of paragraphs 71-113, wherein R^{20a} is C₁₋₃alkyl substituted with 1 or more independent occurrences of deuterium.
115. The compound according to paragraph 114, wherein R^{20a} is methyl substituted with 1-3 independent occurrences of deuterium.
116. The compound according to paragraph 115, wherein R^{20a} is —CD₃.
117. The compound according to any one of paragraphs 71-113, wherein R^{20a} is —CH₃.
118. The compound according to any one of paragraphs 71-117, wherein R^{20b} is deuterium.
119. The compound according to any one of paragraphs 71-117, wherein R^{20b} is hydrogen.
120. The compound according to any one of paragraphs 71-119, wherein each of R^{21a} and R^{21b} is independently deuterium.
121. The compound according to any one of paragraphs 71-119, wherein each of R^{21a} and R^{21b} is independently hydrogen.
122. The compound according to any one of paragraphs 71-121, wherein each of R^{23a} and R^{23b} is independently deuterium.
123. The compound according to any one of paragraphs 71-121, wherein each of R^{23a} and R^{23b} is independently hydrogen.
124. The compound according to any one of paragraphs 71-123, wherein R^{24a} is C₁₋₃alkyl substituted with 1 or more independent occurrences of deuterium.
125. The compound according to paragraph 124, wherein R^{24a} is methyl substituted with 1-3 independent occurrences of deuterium.
126. The compound according to paragraph 125, wherein R^{24a} is —CD₃.
127. The compound according to any one of paragraphs 71-123, wherein R^{24a} is —CH₃.
128. The compound according to paragraph 71 wherein the compound of formula (I) is a compound of formula (IV)

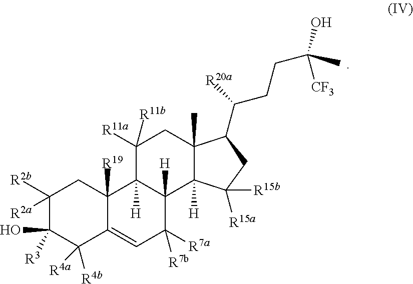

129. The compound according to paragraph 128, wherein the compound of Formula (IV) is selected from the group consisting of any one of compounds A-J;
   wherein any atom not labeled as deuterium is present at its natural isotopic abundance.
130. The compound according to paragraph 71 wherein the compound of formula (I) is a compound of formula (V)

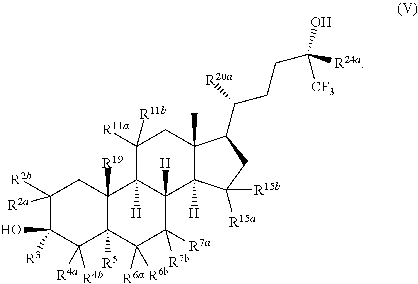

131. The compound according to paragraph 130, wherein the compound of Formula (V) is selected from the group consisting of any one of compounds K-X;
   wherein any atom not labeled as deuterium is present at its natural isotopic abundance.
132. A pharmaceutical composition comprising a compound according to any one of paragraphs 71-131, and a pharmaceutically acceptable carrier.
133. A method for treating a CNS-related condition in a subject, comprising administering to the subject an effective amount of a compound according to any one of paragraphs 71-131, or a pharmaceutical composition according to paragraph 132.
134. The method according to paragraph 133, wherein the CNS-related condition is selected from the group consisting of an adjustment disorder, anxiety disorder (including obsessive-compulsive disorder, post-traumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.
135. A method of inducing sedation or anesthesia in a subject, comprising administering to the subject an effective amount of a compound according to any one of paragraphs 71-131, or a pharmaceutical composition according to paragraph 132.
136. A compound according to any one of paragraphs 71-131, or a pharmaceutical composition according to paragraph 132, for use in treating a CNS-related condition in a subject.
137. The compound or pharmaceutical composition for use according to paragraph 135, wherein the CNS-related condition is selected from the group consisting of an adjustment disorder, anxiety disorder (including obsessive-compulsive disorder, post-traumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.
138. A compound according to any one of paragraphs 71-131, or a pharmaceutical composition according to paragraph 132, for use inducing sedation or anesthesia in a subject.
139. Use of a compound according to any one of paragraphs 71-131, or a pharmaceutical composition according to paragraph 132, for the manufacture of a medicament for treating a CNS-related condition in a subject.
140. The use according to paragraph 139, wherein the CNS-related condition is selected from the group consisting of an adjustment disorder, anxiety disorder (including obsessive-compulsive disorder, post-traumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

What is claimed is:

1. A compound of Formula (I): 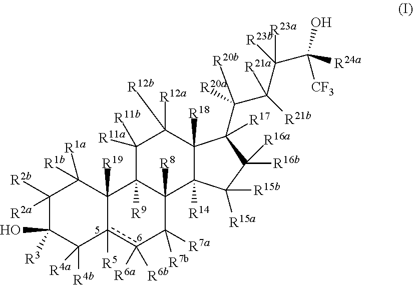
or a pharmaceutically acceptable salt thereof;
wherein:
each of R³, R¹⁸, R¹⁹, R^{20a} and R^{24a} is independently C₁₋₃alkyl optionally substituted with 1 or more independent occurrences of deuterium;
each of R^{1a}, R^{1b}, R^{2a}, R^{2b}, R^{4a}, R^{4b}, R⁵, R^{6a}, R^{6b}, R^{7a}, R^{7b}, R⁸, R⁹, R^{11a}, R^{11b}, R^{12a}, R^{12b}, R¹⁴, R^{15a}, R^{15b}, R^{16a}, R^{16b}, R¹⁷, R^{20b}, R^{21a}, R^{21b}, R^{23a}, and, R^{23b} is independently selected from a group consisting of hydrogen and deuterium;
provided that at least one of R³, R¹⁸, R¹⁹, R^{20a} and R^{24a} is substituted with 1 or more independent occurrences of deuterium, or at least one of R^{1a}, R^{1b}, R^{2a}, R^{2b}, R^{4a}, R^{4b}, R⁵, R^{6a}, R^{6b}, R^{7a}, R^{7b}, R⁸, R⁹, R^{11a}, R^{11b}, R^{12a}, R^{12b}, R¹⁴, R^{15a}, R^{15b}, R^{16a}, R^{16b}, R¹⁷, R^{20b}, R^{21a}, R^{21b}, R^{23a}, and R^{23b} is deuterium; [symbol]
represents a single or double bond; provided if the bond between C5 and C6 is a single bond, then R⁵ is in the alpha configuration.

2. The compound of claim 1, wherein the compound of Formula (I) is a compound of Formula (Ia): 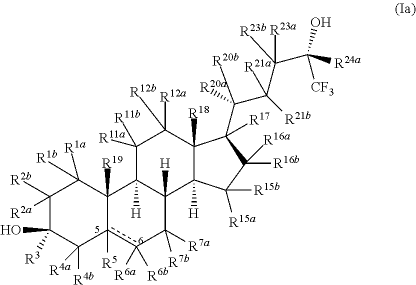
or a pharmaceutically acceptable salt thereof;
wherein:
each of R³, R¹⁸, R¹⁹, R^{20a} and R^{24a} is independently C₁₋₃alkyl optionally substituted with 1 or more independent occurrences of deuterium;
each of R^{1a}, R^{1b}, R^{2a}, R^{2b}, R^{4a}, R^{4b}, R⁵, R^{6a}, R^{6b}, R^{7a}, R^{7b}, R^{11a}, R^{11b}, R^{12a}, R^{12b}, R^{15a}, R^{15b}, R^{16a}, R^{16b}, R¹⁷, R^{20b}, R^{21a}, R^{21b}, R^{23a}, and R^{23b} is independently selected from a group consisting of hydrogen and deuterium;
provided that at least one of R³, R¹⁸, R¹⁹, R^{20a} and R^{24a} is substituted with 1 or more independent occurrences of deuterium, or at least one of R^{1a}, R^{1b}, R^{2a}, R^{2b}, R^{4a}, R^{4b}, R⁵, R^{6a}, R^{6b}, R^{7a}, R^{7b}, R^{11a}, R^{11b}, R^{12a}, R^{12b}, R^{15a}, R^{15b}, R^{16a}, R^{16b}, R¹⁷, R^{20b}, R^{21a}, R^{21b}, R^{23a}, and R^{23b} is deuterium; [symbol]
represents a single or double bond; provided if the bond between C5 and C6 is a single bond, then R⁵ is in the alpha configuration.

3. The compound of claim 2, wherein the compound of Formula (Ia) is a compound of Formula (II): 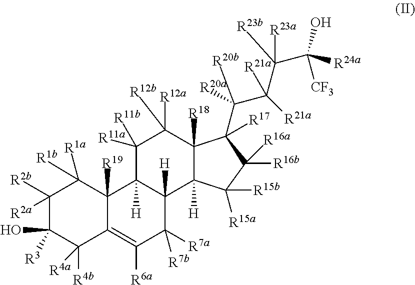
or a pharmaceutically acceptable salt thereof;

4. The compound of claim 2, wherein the compound of Formula (Ia) is a compound of Formula (III): 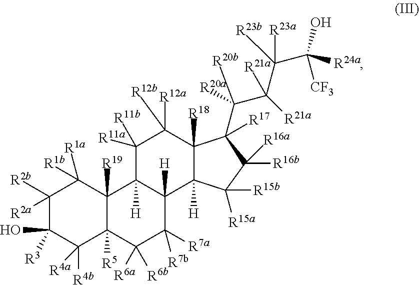
or a pharmaceutically acceptable salt thereof;

5. The compound or pharmaceutically acceptable salt thereof according to any one of claims 1-4, wherein at least one of R^{3a}, R¹⁸, R¹⁹, R^{20a}, or R^{24a} is C₁₋₃ alkyl substituted with 1 or more independent occurrences of deuterium, or at least one of R^{2a}, R^{2b}, R^{4a}, R^{4b}, R⁵, R^{6a}, R^{7a}, R^{7b}, R^{11a}, R^{11b}, R^{15a} and R^{15b} is deuterium.

6. The compound or a pharmaceutically acceptable salt according to any one of claims 1-5, wherein each of R^{1a} and R^{1b} is independently deuterium.

7. The compound or a pharmaceutically acceptable salt according to any one of claims 1-6, wherein each of R^{2a} and R^{2b} is independently deuterium.

8. The compound or pharmaceutically acceptable salt thereof according to any one of claims 1-7, wherein R³ is C₁₋₃alkyl substituted with 1 or more independent occurrences of deuterium.

9. The compound or a pharmaceutically acceptable salt according to any one of claims 1-8, wherein each of R^{4a} and R^{4b} is independently deuterium.

10. The compound or a pharmaceutically acceptable salt according to any one of claims 1-9, wherein R⁵ is deuterium.

11. The compound or a pharmaceutically acceptable salt according to any one of claims 1-10, wherein each of R⁵ and R^{6a} is independently deuterium.

12. The compound or a pharmaceutically acceptable salt according to any one of claims 1 or 3-11, wherein each of R^{6a} and R^{6b} is independently deuterium.

13. The compound or a pharmaceutically acceptable salt according to any one of claims 1-12, wherein each of R^{7a} or R^{7b} is independently deuterium.

14. The compound or a pharmaceutically acceptable salt according to any one of claims 1-13, wherein each of R^{11a} and R^{11b} is independently deuterium.

15. The compound or a pharmaceutically acceptable salt according to any one of claims 1-14, wherein each of R^{12a} and R^{12b} is independently deuterium.

16. The compound or a pharmaceutically acceptable salt according to any one of claims 1-15, wherein each of R^{15a} and R^{15b} is independently deuterium.

17. The compound or a pharmaceutically acceptable salt according to any one of claims 1-16, wherein each of R^{16a} and R^{16b} is independently deuterium.

18. The compound or a pharmaceutically acceptable salt according to any one of claims 1-17, wherein R¹⁷ is deuterium.

19. The compound or pharmaceutically acceptable salt thereof according to any one of claims 1-18, wherein R¹⁸ is C₁₋₃alkyl substituted with 1 or more independent occurrences of deuterium.

20. The compound or pharmaceutically acceptable salt thereof according to any one of claims 1-19, wherein R¹⁹ is C₁₋₃alkyl substituted with 1 or more independent occurrences of deuterium.

21. The compound or a pharmaceutically acceptable salt according to any one of claims 1-20, wherein R^{20a} is C₁₋₃alkyl substituted with 1 or more independent occurrences of deuterium.

22. The compound or a pharmaceutically acceptable salt according to any one of claims 1-21, wherein R^{20b} is deuterium.

23. The compound or a pharmaceutically acceptable salt according to any one of claims 1-22, wherein each of R^{21a} and R^{21b} is independently deuterium.

24. The compound or a pharmaceutically acceptable salt according to any one of claims 1-23, wherein each of R^{23a} and R^{23b} is independently deuterium.

25. The compound or a pharmaceutically acceptable salt according to any one of claims 1-24, wherein R^{24a} is C₁₋₃alkyl substituted with 1 or more independent occurrences of deuterium.

26. The compound of claim 1 wherein the compound of formula (I) is a compound of formula (IV) 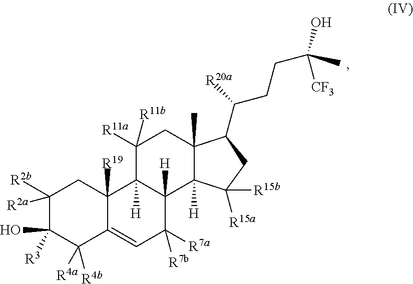
or a pharmaceutically acceptable salt thereof.

27. The compound or pharmaceutically acceptable salt thereof according to claim 26, wherein the compound of Formula (IV) is selected from the group consisting of any one of compounds A-J:

| Compound | | | | | | | | | | | | | | |
|---|---|---|---|---|---|---|---|---|---|---|---|---|---|---|
| Number | R^{2a} | R^{2b} | R³ | R^{4a} | R^{4b} | R^{7a} | R^{7b} | R^{11a} | R^{11b} | R^{15a} | R^{15b} | R¹⁹ | R^{20a} | R^{24a} |
| A | D | D | CH₃ | H | H | H | H | H | H | H | H | CH₃ | CH₃ | CH₃ |
| B | H | H | CD₃ | H | H | H | H | H | H | H | H | CH₃ | CH₃ | CH₃ |
| C | H | H | —CH₃ | D | D | H | H | H | H | H | H | CH₃ | CH₃ | CH₃ |
| D | H | H | —CH₃ | H | H | D | D | H | H | H | H | CH₃ | CH₃ | CH₃ |
| E | H | H | —CH₃ | H | H | H | H | D | D | H | H | CH₃ | CH₃ | CH₃ |
| F | H | H | —CH₃ | H | H | H | H | H | H | D | D | CH₃ | CH₃ | CH₃ |
| G | H | H | —CH₃ | H | H | H | H | H | H | H | H | CD₃ | CH₃ | CH₃ |
| H | H | H | —CH₃ | H | H | H | H | H | H | H | H | CH₃ | CD₃ | CH₃ |
| I | H | H | —CH₃ | H | H | H | H | H | H | H | H | CH₃ | CH₃ | CD₃ |
| J | D | D | —CH₃ | D | D | H | H | H | H | H | H | CH₃ | CH₃ | CH₃ |

or a pharmaceutically acceptable salt thereof;
wherein any atom not labeled as deuterium is present at its natural isotopic abundance.

28. The compound of claim 1 wherein the compound of formula (I) is a compound of formula (V) 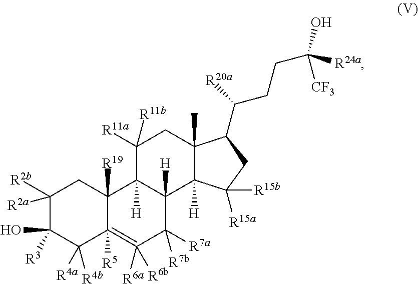
or a pharmaceutically acceptable salt thereof.

29. The compound of claim 28, wherein the compound of Formula (V) is selected from the group consisting of any one of compounds K-X:

| Cmpd | | | | | | | | | | | | | | | | | |
|---|---|---|---|---|---|---|---|---|---|---|---|---|---|---|---|---|---|
| No | R^{2a} | R^{2b} | R³ | R^{4a} | R^{4b} | R⁵ | R^{6a} | R^{6b} | R^{7a} | R^{7b} | R^{11a} | R^{11b} | R^{15a} | R^{15b} | R¹⁹ | R^{20a} | R^{24a} |
| K | H | H | CH₃ | H | H | D | D | H | H | H | H | H | H | H | CH₃ | CH₃ | CH₃ |
| L | D | D | CH₃ | H | H | H | H | H | H | H | H | H | H | H | CH₃ | CH₃ | CH₃ |
| M | H | H | CD₃ | H | H | H | H | H | H | H | H | H | H | H | CH₃ | CH₃ | CH₃ |
| N | H | H | CH₃ | D | D | H | H | H | H | H | H | H | H | H | CH₃ | CH₃ | CH₃ |
| O | H | H | CH₃ | H | H | H | H | D | H | H | H | H | H | H | CH₃ | CH₃ | CH₃ |
| P | H | H | CH₃ | H | H | H | H | H | D | D | H | H | H | H | CH₃ | CH₃ | CH₃ |
| Q | H | H | CH₃ | H | H | H | H | H | H | H | D | D | H | H | CH₃ | CH₃ | CH₃ |
| R | H | H | CH₃ | H | H | H | H | H | H | H | H | H | D | D | CH₃ | CH₃ | CH₃ |
| S | H | H | CH₃ | H | H | H | H | H | H | H | H | H | H | H | CD₃ | CH₃ | CH₃ |
| T | H | H | CH₃ | H | H | H | H | H | H | H | H | H | H | H | CH₃ | CD₃ | CH₃ |
| U | H | H | CH₃ | H | H | H | H | H | H | H | H | H | H | H | CH₃ | CH₃ | CD₃ |
| V | H | H | CH₃ | H | H | D | H | H | H | H | H | H | H | H | CH₃ | CH₃ | CH₃ |
| W | H | H | CH₃ | H | H | H | D | D | H | H | H | H | H | H | CH₃ | CH₃ | CH₃ |
| X | D | D | CH₃ | D | D | H | H | H | H | H | H | H | H | H | CH₃ | CH₃ | CH₃ |

or a pharmaceutically acceptable salt thereof;
wherein any atom not labeled as deuterium is present at its natural isotopic abundance.

30. A pharmaceutical composition comprising a compound or a pharmaceutically acceptable salt thereof according to any one of claims 1-29, and a pharmaceutically acceptable carrier.

31. A method for treating a CNS-related condition in a subject, comprising administering to the subject an effective amount of a compound according to any one of claims 1-29 or a pharmaceutically acceptable salt thereof, or a pharmaceutical composition according to claim 30.

32. The method according to claim 31, wherein the CNS-related condition is selected from the group consisting of an adjustment disorder, anxiety disorder (including obsessive-compulsive disorder, post-traumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

33. A method of inducing sedation or anesthesia in a subject, comprising administering to the subject an effective amount of a compound according to any one of claims 1-29, or a pharmaceutically acceptable salt thereof, or a pharmaceutical composition according to claim 30.

34. A compound according to any one of claims 1-29 or a pharmaceutically acceptable salt thereof, or a pharmaceutical composition according to claim 30, for use in treating a CNS-related condition in a subject.

35. The compound, pharmaceutically acceptable salt thereof, or pharmaceutical composition for use according to claim 34, wherein the CNS-related condition is selected from the group consisting of an adjustment disorder, anxiety disorder (including obsessive-compulsive disorder, post-traumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

36. A compound according to any one of claims 1-29 or a pharmaceutically acceptable salt thereof, or a pharmaceutical composition according to claim 30, for use in inducing sedation or anesthesia in a subject.

37. Use of a compound according to any one of claims 1-29 or a pharmaceutically acceptable salt thereof, or a pharmaceutical composition according to claim 30, for the manufacture of a medicament for treating a CNS-related condition in a subject.

38. The use according to claim 37, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.
